(19) United States
(12) Patent Application Publication
(10) Pub. No.: US 2022/0412957 A1
(43) Pub. Date: Dec. 29, 2022

(54) METHODS OF TREATING CANCER PATIENTS WITH RAS NODE OR RTK TARGETED THERAPEUTIC AGENTS

(71) Applicant: Celcuity Inc., Minneapolis, MN (US)

(72) Inventors: Lance Gavin LAING, Orono, MN (US); Salmaan Ahmed KHAN, Arden Hills, MN (US); Brian Francis SULLIVAN, Medina, MN (US)

(21) Appl. No.: 17/835,208

(22) Filed: Jun. 8, 2022

Related U.S. Application Data
(63) Continuation of application No. PCT/US2020/063890, filed on Dec. 9, 2020.
(60) Provisional application No. 62/945,608, filed on Dec. 9, 2019.

(51) Int. Cl. G01N 33/50; A61P 35/00; G16H 20/10; C07K 14/72

(52) U.S. Cl. CPC G01N 33/5017; A61P 35/00; G01N 33/5032; G16H 20/10; C07K 14/723; G01N 2800/52; G01N 2800/56

(57) ABSTRACT

Provided herein are methods for determining the functional status of G-protein coupled receptor (GPCR) signaling pathways in a diseased cell sample obtained from a subject to thereby select for therapeutic use in the subject a RAS node or receptor tyrosine kinase (RTK) targeted therapeutic. Also provided are methods for determining whether a GPCR signaling pathway is ultrasensitive in a diseased cell sample from a subject. Methods of administering a selected RAS node or RTK targeted therapeutic agent to the subject are also provided.

### RELATED APPLICATIONS

This application is a continuation of International Application No. PCT/US2020/063890, filed on Dec. 9, 2020, which claims priority to, and the benefit of, U.S. provisional patent application Ser. No. 62/945,608, filed Dec. 9, 2019, the contents of which are hereby incorporated by reference.

### BACKGROUND

Cancer is a significant medical problem, with estimates as high as approximately 40% of men and women in the United States being diagnosed with cancer at some point during their lifetime. Given the heterogeneity of cancers across patients, tailoring therapy based on the characteristics of each patient's cancer and being able to determine whether a patient is likely to respond to a particular therapeutic is of great interest and the objective of personalized medicine. Notwithstanding, the existing prognostic toolset has significant shortcomings. Indeed, detection of current leading genetic and protein biomarkers has been shown to have poor predictive value for therapeutic responsiveness.

One response to the realization that each patient differs and that therapies may often fail to elicit a positive response has been the development of companion diagnostics. This type of diagnostic test is designed using contemporary biomarker detection tools to try to identify those patients who are more likely to respond to a particular drug. The test involves looking for increased gene number, gene mutation(s), or altered expression level of a particular gene. Many cancers today are diagnosed with the aid of tests to determine the presence of specific genetic mutations or over-expressed receptor proteins associated with the disease. However, most biomarker tests only provide indirect and inferential information about the disease and its underlying cause. Thus, the success rates for most of these tests at predicting significant therapeutic response are often much less than 50%. For instance, less than 20% of late-stage breast cancer patients with a PI3KCA mutation respond to alpelisib (Andre et al., N Engl J Med 2019; 380:1929-40), an approved PIK3CA inhibitor, suggesting that the presence of genetic mutations is not necessarily predictive of whether a patient will or will not respond to a drug that specifically targets aberrant signaling effected by the mutated gene/protein.

This observation, that simply detecting the expression of a particular genetic or protein biomarker by a patient's cells is not a highly effective predictor of therapeutic responsiveness, demonstrates that more effective means for selecting therapeutic treatment regimens are needed. One approach to address how a specific patient's cells are functioning, or malfunctioning, has been to examine the activity of a signaling pathway in the patient's cells, through assessment of a change in a physiological response parameter, such as cell adhesion, that indicates activity, or lack thereof, of the signaling pathway. This approach has allowed for accurate prediction of the effectiveness of targeted therapeutic agents that selectively affect the signaling pathway being examined (as described in PCT Publications WO 2013/188500 and WO2018/175251, the contents of which are herein incorporated by reference in their entirety). However, given the limited arsenal of approved drugs on the market, there is always a need to identify new subpopulations of patients who may be responsive and treatable with these drugs.

### SUMMARY

The methods described herein allow for the identification, selection, and treatment of a subpopulation of cancer patients with aberrant G protein coupled receptor (GPCR) signaling who are responsive to inhibition of a RAS node (e.g., phosphoinositide 3-kinase (PI3K), AKT, mTOR, RAF, MEK, ERK, BCL) or a receptor tyrosine kinase (RTK) signaling pathway and thus are selected for treatment with a RAS node or RTK targeted therapeutic, respectively. Such methods are highly beneficial in that they allow for the identification of a subpopulation of patients who may not have genetic mutations in a RAS node or RTK, typically a requirement under standard-of-care guidelines, and thus would not have initially been considered for treatment with these targeted therapeutics, but are newly identified as candidates for treatment using the methods described herein. The methods disclosed herein are particularly suited to identifying, selecting, and treating patients with aberrant lysophospholipid GPCR signaling with RAS node or RTK node involvement.

More specifically, the methods disclosed herein involve activation (agonism) of signaling pathway activity at one site, namely a GPCR, combined with inhibition (antagonism) of signaling pathway activity at another site, namely a RAS node (e.g., PI3K, AKT, mTOR, RAF, MEK, ERK, BCL) or RTK, to thereby determine whether aberrant GPCR signaling pathway activity in the patient's cells can be modulated by inhibition of a RAS node or RTK signaling pathway. As described herein, these methods have successfully identified subpopulations of cancer patients whose cells are responsive to an inhibitor of a RAS node despite having a non-mutated (i.e., wild-type) version of the RAS node targeted by the inhibitor. Moreover, the methods have successfully identified subpopulations of cancer patients whose cells are responsive to an RTK inhibitor despite not over-expressing the RTK (i.e., RTK negative cells). In addition, the methods also have successfully identified subpopulations of cancer patients whose cells are responsive to a PI3K inhibitor despite not having a mutated PI3K enzyme. Still further, the methods also have successfully identified subpopulations of cancer patients whose cells are not responsive to a PI3K inhibitor despite having a mutated PI3K enzyme. Accordingly, the methods of the disclosure make available targeted therapies for certain populations of cancer patients that otherwise would not have been identified under current standards of care. The methods also identify patients who should not be indicated for treatment with a PI3K inhibitor even though current standards of care that use a mutated PI3K enzyme as the selection criteria would identify them for treatment with a PI3K inhibitor.

Accordingly, in one aspect, provided herein is a method of selecting a human subject diagnosed with cancer for treatment with a RAS node targeted therapeutic, the method comprising:

culturing a sample of viable cancer cells obtained from the subject;

contacting (1) a first portion of the sample with an agonist of GPCR signaling and an inhibitor of a RAS node, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

selecting the subject for treatment with a RAS node targeted therapeutic which inhibits the same RAS node as the inhibitor if the output value that characterizes the change in cell adhesion or attachment is equal to or greater than a pre-determined cut-off value, or if the output value percentage is equal to or greater than 30%.

In another aspect, provided herein is a method of treating a human subject diagnosed with cancer mediated by a RAS node, wherein cancer cells of the subject have been tested in a method comprising:

culturing a sample of viable cancer cells obtained from the subject;

contacting (1) a first portion of the sample with an agonist of GPCR signaling and an inhibitor of a RAS node, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

administering to the subject a RAS node targeted therapeutic which inhibits the same RAS node as the inhibitor if the output value that characterizes the change in cell adhesion or attachment is equal to or greater than a pre-determined cut-off value, or if the output value percentage is equal to or greater than 30%.

In some embodiments, the RAS node is selected from the group consisting of phosphoinositide 3 kinase (PI3K), ERK, MEK, mTOR, RAF, BCL, and a combination thereof.

In some embodiments, the first portion of the sample is contacted with two or more inhibitors, each of which inhibits a different RAS node. For instance, the two or more inhibitors may inhibit a combination of RAS nodes selected from the group consisting of: (a) PI3K, mTOR, and BCL, (b) PI3K, mTOR, and RAF, (c) PI3K, mTOR, and ERK, and (d) PI3K, mTOR, and MEK. In some embodiments, the subject is treated or selected for treatment with a combination of RAS node targeted therapeutics which inhibit the different RAS nodes.

In some embodiments, the RAS node is PI3K, and the inhibitor is a PI3K inhibitor. In one embodiment, the PI3K inhibitor selectively inhibits the p110α catalytic subunit of PI3K. In another embodiment, the PI3K inhibitor selectively inhibits the p110β catalytic subunit of PI3K. In another embodiment, the PI3K inhibitor selectively inhibits the p110γ catalytic subunit of PI3K. In yet another embodiment, the PI3K inhibitor selectively inhibits the p110δ subunit of PI3K. In some embodiments, the PI3K inhibitor inhibits more than one isoform of the p110 subunit of PI3K. In some embodiments, the PI3K inhibitor inhibits all isoforms of the p110 catalytic subunit of PI3K. In some embodiments, the PI3K inhibitor also inhibits mTOR.

In some embodiments, the PI3K inhibitor is selected from the group consisting of: wortmannin, LY294002, hibiscone C, Idelalisib (GS-1101, CAL-101), Copanlisib, Duvelisib, Alpelisib, (BYL719), Taselisib (GDC-0032), GDC-0077, Perifosine, Idealisib, Pilaralisib (XL147), Buparlisib (BKM120), Duvelisib, Umbralisib, PX-866, Dactolisib, CUDC-907, Voxtalisib, ME-401, IPI-549, SF1126, RP6530, INK1117, Pictilisib (GDC-0941), Palomid 529, SAR260301, GSK1059615, GSK2636771, CH5132799, CZC24832, AZD6482, AZD8835, WX-037, AZD8186, KA2237, CAL-120, AMG-319, AMG-511, HS-173, INCB050465, INCB040093, TGR-1202, ZSTK474, PWT33597, IC87114, TG100-115, TGX221, CAL263, RP6530, PI-103, GNE-477, IPI-145, BAY 80-6946, BAY1082439, PX866, BEZ235, MKM120, MLN1117, SAR245408, AEZS-136, serabelisib (TAK-117), gedatolisib, omipalisib, and pilarlisib.

In some embodiments, the RAS node is ERK, and the inhibitor is an ERK inhibitor, e.g., an ERK inhibitor selected from the group consisting of: ravoxertinib, SCH772984, SCH900353 (MK8353), ulixertinib, AZD0364 (ATG017), VX-11e (VTX-113), CC-90003, LY3214996, FR180204, ASN007, and GDC0994.

In some embodiments, the RAS node is MEK, and the inhibitor is a MEK inhibitor, e.g., a MEK inhibitor selected from the group consisting of: trametinib, binimetinib, pimasertib, cobimetinib, PD901, U0126, selumetinib, PD325901, TAK733, CI-1040 (PD184352), PD198306, PD334581, PD98059, and SL327.

In some embodiments, the RAS node is mTOR, and the inhibitor is a mTOR inhibitor, e.g., a mTOR inhibitor selected from the group consisting of: gedatolisib, omipalisib, sirolimus, everolimus, temsirolimus, dactolisib, AZD8055, ABTL-0812, PQR620, GNE-493, KU0063794, torkinib, ridaforolimus, sapanisertib, voxtalisib, torin 1, torin 2, OSI-027, PF-04691502, apitolisib, GSK1059615, WYE-354, vistusertib, WYE-125132, BGT226, palomid 529, WYE-687, WAY600, GDC-0349, XL388, bimiralisib (PQR309), onatasertib (CC-223), samotolisib, and GNE-477.

In some embodiments, the RAS node is RAF, and the inhibitor is a RAF inhibitor, e.g., a RAF inhibitor selected from the group consisting of: PLX7904, GDC-0879, belvarafenib (GDC-5573), SB590885, encorafenib, RAF265, RAF709, dabrafenib (GSK2118436), TAK-632, TAK580, PLX-4720, CEP-32796, sorafenib, vemurafenib (PLX-4032), AZ-628, GW5074, ZM-336372, NVP-BHG712, CEP32496, PLX4032, and LGX-818.

In some embodiments, the RAS node is Bcl-xL, and the inhibitor is a Bcl-xL inhibitor, e.g., a Bcl-xL inhibitor selected from the group consisting of: navitoclax (ABT-263), GDC-0199 (ABT-199), sabutoxlax (B1-97C1), ABT-737, AT101, TW037, A1331852, BXI-61, and BXI-72.

In some embodiments, the RAS node inhibitor and RAS node targeted therapeutic are the same compound. In other embodiments, the RAS node inhibitor and RAS node targeted therapeutic are different compounds.

In another aspect, provided herein is a method of selecting a human subject diagnosed with cancer for treatment with an RTK targeted therapeutic, the method comprising:

culturing a sample of viable cancer cells obtained from the subject;

contacting (1) a first portion of the sample with an agonist of GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

selecting the subject for treatment with an RTK targeted therapeutic which inhibits the same RTK as the inhibitor of RTK signaling if the output value that characterizes the change in cell adhesion or attachment is equal to or greater than a pre-determined cut-off value, or if the output value percentage is equal to or greater than 30%.

In another aspect, provided herein is a method of treating a human subject diagnosed with cancer mediated by RTK signaling, wherein cancer cells of the subject have been tested in a method comprising:

culturing a sample of viable cancer cells obtained from the subject;

contacting (1) a first portion of the sample with an agonist of GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

administering to the subject an RTK targeted therapeutic which inhibits the same RTK as the inhibitor of RTK signaling if the output value that characterizes the change in cell adhesion or attachment is equal to or greater than a pre-determined cut-off value, or if the output value percentage is equal to or greater than 30%.

In some embodiments, the RTK is selected from a member of the EGFR/ERBB, MuSK, HGFR, NGFR, FGFR, IR, CCK, EphR, RYK, RET, ROS, PDGFR, DDR, LTK, VEGFR, TIE, AXL, ROR, LMR, or RTK106 family. In one embodiment, the RTK is a member of the ErbB family, for example, HER2.

In some embodiments, the RTK inhibitor is selected from the group consisting of: erlotinib, gefitinib, lapatinib, vandetanib, afatinib, panitumumab, cetuximab, brigatinib, icotinib, osimertinib, neratinib, zalutumumab, nimotuzumab, matuzumab, pertuzumab, trastuzumab, dacomitinib, acomitinib, BIBW2992, tesevatinib, amuvatinib, necitumumab, REGN955, MM-151, nazartinib, ASP8273, olmutinib, TDM1, MEDI4276, ZW25, ZW33, tucatinib, rociletinib, ibrutinib, DS-8201, TAS07828, XMT-1522, TAK-788, Sym013, LIM716, seribantumab, AMG888, lumretuzumab, PF-06804103, ARX788, poziotinib, pyrotinib, duligotuzuman, MCLA-128, MM-111, cabozantinib, tivantinib, crizotinib (PF-2341066), tepotinib, capmatinib, savolitinib, K252a, SU11274, PHA-665752, ARQ197, foretinib, SGX523, MP470, AV229, AMG102, CGEN241, DN30, OA5D5, rilotumumab, onartuzumab, SAR125844, embetuzumab, ABBV-399, sym015, ficlatuzumab, merestinib, JNJ-61186372, altiratinib, Indo5, BMS-754807, BMS-777607, glesatinib, CEP-751, ANA-12, cyclotraxin B, gossypetin, entrectinib, larotrectinib, LOXO-101, dovotinib, lenvatinib, ponatinib, regorafenib, lucitanib, cediranib, intedanib, brivanib, PD173074, AZD4547, BGJ398, JNJ42756493, GP369, BAY1187982, MFGR1877S, FP1039, pazopanib, erdafitinib, Debio-1347, B-701, fisogatinib, FIIN-2, FIIN-3, BLU9931, LY2874455, LY3076226, sunitinib, AG538, AG1024, NVP-AEW541, figitumumab, linsitinib, dalotuzumab, MEDI-573, teprotumumab, ganitumab, ceritinib, MM-141, cofetuzumab pelidotin (PF-06647020), dasatinib, nilotinib, NVP-BHG712, sitravatinib, ALW-II-41-27, JI-101, 123C4, sorafenib, apatinib, AST487, alectinib, dovitinib, crizotinib, lorlatinib, TPX-0005, DS-6051b, imatinib, linifanib, KTN0182A, gilteritinib, quizartinib, midostaurin, lestaurtinib, ripretinib, masitinib, avapritinib, pexidartinib, telatinib, motesanib, PLX7486, ARRY386, JNJ-40346527, BLZ945, emactuzumab, AMG820, IMC-CS4, cabiralizumab, CHMFL-KIT-033, SU14813, Ki20227, OSI-930, flumatinib, toceranib, AZD3229, AC710, AZD2932, ICK03, PLX647, c-Kit-IN-3, vatalanib, bevacizumab, rebastinib, BAY-826, bemcentinib, R428 (BGB324), YW327.6S2, GL2I.T, TP-0903, LY2801653, bosutinib, MGCD265, ASP2215, SGI-7079, BGB324, HuMax-AXL-ADC, and UC-961. In some embodiments, the RTK inhibitor and RTK targeted therapeutic are the same compound. In other embodiments, the RTK inhibitor and RTK targeted therapeutic are different compounds.

In another aspect, provided herein is a method of determining whether a human subject diagnosed with cancer has abnormally active GPCR signaling comprising,

culturing a sample comprising viable cancer cells obtained from the subject;

contacting the sample with an agonist of a GPCR signaling pathway;

continuously measuring cell adhesion or attachment of the viable cancer cells in a portion of the sample contacted with the agonist relative to a portion of the sample that has not been contacted with the agonist; and

determining by mathematical analysis of the continuous measurements sensitivity of the sample to the agonist and an output value for the agonist that characterizes whether a change in cell adhesion or attachment has occurred in the portion of the sample contacted with the agonist, as compared to the portion of the sample not contacted with the agonist, wherein the GPCR signaling pathway is considered abnormally active when the output value for the agonist is equal to or greater than a pre-determined cut-off value.

In another aspect, provided herein is a method of determining whether a human subject diagnosed with cancer has abnormally active GPCR signaling comprising,

culturing a sample comprising viable cancer cells obtained from the subject;

contacting the sample with an agonist of a GPCR signaling pathway, wherein a portion of the sample is contacted with a higher concentration of the agonist and a portion of the sample is contacted with a lower concentration of the agonist;

continuously measuring cell adhesion or attachment of the viable cancer cells in the portion of the sample contacted with a higher concentration of the agonist, relative to the portion of the sample contacted with the lower concentration of the agonist; and

determining by mathematical analysis of the continuous measurements the sensitivity of the signaling pathway to the agonist, wherein the GPCR signaling pathway is considered abnormally active when the signaling pathway is ultra-sensitive to the agonist.

In some embodiments, the higher concentration of the agonist is EC90 and the lower concentration of the agonist is EC10. In one embodiment, an EC90:EC10 ratio of less than 81 indicates that the signaling pathway is ultra-sensitive to the agonist. In some embodiments, sensitivity of the signaling pathway to the agonist is determined using the Hill equation to determine a Hill Coefficient. In one embodiment, a Hill Coefficient value of greater than one indicates that the signaling pathway is ultra-sensitive to the agonist.

In some embodiments of the methods described above, the GPCR is a lysophospholipid GPCR, such as an LPA receptor (e.g., LPAR1, LPAR2, LPAR3, LPAR4, LPAR5, and LPAR6) or a S1P receptor (e.g., S1PR1, S1PR2, S1PR3, S1PR4, and S1PR5).

In some embodiments, the agonist of GPCR signaling is a protein, peptide, lipid, nucleic acid, metabolite, ligand, reagent, organic molecule, signaling factor, growth factor, biochemical, or combinations thereof. Suitable agonists of LPA receptors include, for example, lysophosphatidic acid (LPA) (e.g., LPA 18:0, LPA 18:1, LPA 18:2, LPA 20:4, and LPA 16:0), FAP-10, FAP-12, and OMPT. Suitable agonists of SIP receptors include, for example, sphingosine-1-phosphate (S1P), FTY720, BAF312, A971432, ceralifimod, CS2100, CYM50260, CYM50308, CYM5442, CYM5520, CYM5541, GSK2018682, RP001 hydrochloride, SEW2871, TC-G1006, and TC-SP14.

In some embodiments of the methods described above, the GPCR signaling pathway is abnormally active in the subject's cancer cells. In some embodiments, the cancer is selected from the group consisting of breast cancer, lung cancer, colorectal cancer, bladder cancer, kidney cancer, ovarian cancer and leukemia. In one embodiment, the RAS node (e.g., PI3K enzyme or gene) in cancer cells obtained from the subject is non-mutated (wild-type), e.g., a non-mutated PI3K, RAF, MEK, ERK, mTOR, BCL, or a combination thereof. In another embodiment, the RAS node (e.g., PI3K enzyme or gene) in the cancer cells obtained from the subject is mutated. In another embodiment, the cancer cells obtained from the subject do not overexpress the RTK, such as HER2 (i.e., the cancer cells are RTK (e.g., HER2) negative cancer cells) or have genetic mutation associated with the RTK (e.g. HER2 gene amplification). In another embodiment, the GPCR (e.g., LPA receptor or S1P receptor) activated by the agonist of GPCR signaling in the cancer cells is not mutated. In another embodiment, the GPCR activated by the agonist of GPCR signaling in the cancer cells is not over-expressed.

In some embodiments, the sample of viable cancer cells is cultured in a media comprising growth factors and free of serum. In some embodiments, the sample of viable cancer cells is also cultured in a media comprising an anti-apoptotic agent and free of serum.

In some embodiments of the methods described above, cell adhesion or attachment is measured using an impedance biosensor or an optical biosensor.

### DETAILED DESCRIPTION

The present disclosure provides methods for identifying cancer patients whose cells are responsive to targeted therapeutic agents, as well as methods of selecting and treating such patients with such agents. The disclosure is based, at least in part, on the discovery that patients responsive to RAS node (e.g., PI3K, AKT, mTOR, RAF, MEK, ERK, and BCL) or RTK targeted therapeutics can be identified in a cellular-based assay that uses changes in cell adhesion or attachment as the read-out by activating a GPCR signaling pathway in the cells, coupled with inhibition of the RAS node (e.g., PI3K, AKT, mTOR, RAF, MEK, ERK, and BCL) or RTK signaling pathways. Thus, the patient's cells are agonized at one site, namely the GPCR, and inhibited at another site, namely a RAS node or nodes or an RTK, which has been shown to successfully identify patient cancer cells that are responsive to RAS node or RTK inhibitors. Moreover, responsive cancer cells have been successfully identified both in patients that express non-mutated RAS nodes (e.g., wild-type PI3K, mTOR, RAF, MEK, and ERK) and in patients that express mutated PI3K enzyme or genes, as well as in patients whose cells do not overexpress the RTK (i.e., RTK-negative cancer cells, such as HER2-negative cancer cells) or have genetic mutation associated with the RTK (e.g. HER2 gene amplification). Thus, the methods of the present disclosure allow for the identification of new subpopulations of cancer patients for treatment with targeted therapeutic agents.

In order that the present disclosure may be more readily understood, certain terms are first defined. Additional definitions are set forth throughout the detailed description. Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference. The following terms, as used herein, are intended to have the following definitions.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5).

The terms “activator,” “activate,” “activation,” “perturbant,” “perturb,” and “perturbation,” when used in conjunction with reference to cells, refer to the specific subject or activity of physiologic manipulation of a cell using reagents, approved drugs, experimental compounds and drug like molecules and experimental drugs in development, organic molecules, growth factors, signaling factors, biochemicals, nucleic acids, cytokines, chemokines, or proteins that have an effect on cells well known to those practiced in the art. The manipulation refers to any modulation of cellular physiologic activity and may include but not be limited to up or down-regulation. As used herein, the term “agonism” refers to up-regulation of cellular physiologic activity (e.g., cell signaling activity), wherein the term “antagonism” refers to down-regulation of cellular physiologic activity (e.g., cell signaling activity).

The term “adhesion” can encompass processes involving any number of molecules responsible for connecting a cell to an ECM or to other cells directly, indirectly, and or indirectly by pathway communication. For example, integrins are responsible for cytoskeletal organization, cellular motility, regulation of the cell cycle, regulation of cellular of integrin affinity, attachment of cells to viruses, attachment of cells to other cells or ECM. Integrins are also responsible for signal transduction, a process whereby the cell transforms one kind of signal or stimulus into another intra- and inter-cellularly. Integrins can transduce information from the ECM to the cell and information from the cell to other cells (e.g., via integrins on the other cells) or to the ECM. The combination of the α- and β-subunits determines the ligand specificity of the integrin. Many integrins have binding specificities for the same ligands and it is the combination of the integrin expression/activation pattern and the availability of ligand that specifies the interactions in vivo. Adhesion can change in density within a cell area or area of a population of cells. Adhesion can change in quantity within a cell or population of cells. Adhesion can change in quality by specificity or protein types involved in the adhesion process. Adhesion can change in polarity.

The term “assay” or “assaying” refers to an analysis to determine, for example, the presence, absence, quantity, extent, kinetics, dynamics, or type of a target, such as a cell's optical or bioimpedance response upon activation with exogenous stimuli (e.g., therapeutic agent or ligand).

The term “attach” or ““attachment” refers to, for example, a surface modifier substance, a cell, a ligand candidate compound, and like entities of the disclosure, connected to a surface, such as by physical absorption, chemical bonding, chemical attraction, and like processes, or combinations thereof. Particularly, “cell attachment,” “cell adhesion,” or “cell sample attachment” refer to the binding of cells together or interacting to a surface, such as by culturing, or interacting with a cell anchoring material, or the like.

The term “attachment pattern” refers to observable traits or characteristics of a cell or cell sample's connection to a surface. An attachment pattern can be quantitative, e.g., number of attachment sites. An attachment pattern can also be qualitative, e.g., preferred molecular site of attachment to an extracellular matrix.

The term “Cell Attachment Signal (CAS)” refers to a quantitative measurement of cell attachment generated by cells when placed in the well of a microplate and analyzed with an impedance biosensor. Typically, a cell's CAS in the absence of any agents can be compared to the cell's CAS in the presence of an activator agent (e.g., an agonist such as an agonist of a GPCR signaling pathway) alone that affects a particular signaling pathway and/or to the cell's CAS in the presence of an activator agent that affects a particular signaling pathway in combination with a specific inhibitor of that particular signaling pathway or a node in the pathway. Typically, a cell's CAS is measured in ohms.

The term “antibody” is used in the broadest sense and specifically includes monoclonal antibodies (including full length monoclonal antibodies), humanized antibodies, chimeric antibodies, multispecific antibodies (e.g., bispecific antibodies), and antibody fragments that exhibit a desired biological activity or function.

Antibodies can be chimeric, humanized, or human, for example, and can be antigen-binding fragments of these. “Antibody fragments” comprise a portion of a full-length antibody, generally the antigen binding or variable region thereof. Examples of antibody fragments include Fab, Fab′, F(ab′)2, and Fv fragments; diabodies; linear antibodies; single-chain antibody molecules; and multispecific antibodies such as bispecific antibodies, for example formed from antibody fragments. “Functional fragments” substantially retain binding to an antigen of the full-length antibody, and retain a biological activity. Antibodies can be “armed” or “conjugated” by combining with one or more other drugs through covalent or other attachment to achieve greater potency, specificity, and efficacy than the individual drug molecules could achieve separately.

The term “confirming agent” as used herein refers to a small molecule, specific ligand of known function, or antibody or affinity/specificity reagent, known to disrupt or affect the pathway activity of interest that is employed in the test described herein. It is used in the test to confirm and quantify the amount of pathway activity associated with a specific mechanism of action generated when an activator agent is introduced to a cell sample that specifically initiates an activity directly associated with the pathway activity of interest. For instance, if an activator is a known ligand for a cell surface receptor, the activity measured in the method described herein that is changed after introduction of the confirming agent would represent the amount of activity associated with the pathway the method is intended to analyze. In a further example, as may be the case with a receptor comprised of ligand binding region, a receptor dimerization region, and a receptor tyrosine kinase region, an activator agent could be the ligand that binds to the receptor ligand binding site. A confirming reagent then could be an agent that prevented the event(s) preceding the ligand binding or subsequent to the ligand binding i.e. the receptor dimerization or the receptor tyrosine kinase activity subsequent to the receptor dimerization. Furthermore, the confirming reagent could be an agent that refines a particular part of the downstream signaling pathway activated or dysfunctional to the patient of interest.

The term “monoclonal antibody” as used herein refers to an antibody obtained from a population of substantially homogeneous antibodies, i.e., the individual antibodies of the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method.

The term “immunocapture reagents” refers to any type of antibody and additionally includes aptamers composed of RNA, DNA, and polymers containing synthetic variants of bases, or any synthetic molecule where the aptamer or reagent has been constructed and selected to specifically recognize and bind another molecule and signal its presence, quantity, and or quality.

The term “culturing” refers to preparation of cells to perform the present invention. The preparation can include at different times in the practice of the current invention, various media, media supplements, various conditions of temperature, humidity, CO2%, seed densities, cell type purity or mixtures and other conditions known to those practiced in the art of cell culture. The preparation may include conditions that allow the cells to proliferate, become quiescent, senesce, and enter, pass or are checked at various stages of cell cycle. The culturing may include any number of media or supplements known to those practiced in the art such as but not limited to vitamins, cytokines, growth factors, serums (Ex. source animal is bovine, fetal bovine, human, horse or other mammal), metabolites, amino acids, trace minerals, ions, pH buffers, and or glucose, that allow and or optimize the ideal practice of the present invention. Culturing the cells may be practiced with serum-free and or activator-free media before or following activation by the present invention. The culturing may ideally comprise conditions designed to mimic the tumor microenvironment of the patient. The culturing preparation may ideally comprise conditions that are designed to place particular pathways into a basal or heightened level to permit the measurement of agonism or antagonism of the pathway activity.

The term “base media” refers to a type of culture media that contains, in well-defined amounts, inorganic salts, essential amino acids, glucose, vitamins, and pH buffer and it does not contain agents that stimulate the signaling pathway the method is intended to analyze. Many base media are known to those practiced in the art and can include for example DMEM, F12, MEM, MEGM, RPMI-1640 and combinations thereof. For example, when the ErbB signaling pathways are to be analyzed, the base media does not contain reagents known to perturb the ErbB pathways. Base media is used to maintain a cell culture such that the cell population remains viable and retains its heterogeneity of individual cell types and a normal distribution of cells representing the different phases of the cell cycle. It is used to culture a sample of diseased cells obtained from a subject in the methods described herein just prior to the step in the methods where a sample of disease cells are contacted with activator agents.

The term “fresh” when applied to a material refers to a material that has not yet been used. Fresh base media is thus base media that has not yet been used. Fresh base media can be added to a vessel containing a sample of disease cells already being cultured in base media to increase the volume of base media in the vessel containing the cell culture. Alternatively, a portion of the base media that has been used to culture a sample of diseased cells in a vessel can be removed from the cell culture vessel and replaced with fresh base media. In either case, when more than 50% of the total base media volume in a cell culture vessel is fresh base media, the cell culture vessel is considered to contain fresh base media. Cells cultured in the same base media for extended periods of time (e.g. more than 72 hours) will lose the heterogeneity of individual cell types and the majority of the cells may enter G0/G1 cell cycle phase which may interfere with the measurement of signaling pathway activity. A cell sample placed in fresh media requires a period of time (e.g. at least 12 hours) to adjust to the new media such that the cell sample reflects the heterogeneity of individual cell types found in the original cell sample and a normal distribution of cells representing the different phases of the cell cycle.

The term “buffer media” refers to a solution that contains pH buffer and an isotonic solution. Buffer media is typically used to starve a sample of cells or drive the cells into quiescence or senescence such as one finds when cells are resting is cell-cycle G0/G1.

“Chimeric” antibodies (immunoglobulins) contain a portion of a heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., 1984, Proc. Natl. Acad. Sci. USA 81:6851-6855).

The term “humanized antibody” refers to antibodies that contain minimal sequence derived from nonhuman immunoglobulin. For the most part, humanized antibodies are human immunoglobulins (recipient or acceptor antibody) in which variable domain hypervariable region residues of the recipient antibody are replaced by hypervariable region residues from a nonhuman species (donor antibody), such as mouse, rat, rabbit, or nonhuman primate having the desired specificity, affinity, and capacity. The hypervariable regions can be complementarity-determining regions (CDRs) defined by sequence (see, for example Kabat 1991, 1987, 1983), or hypervariable loops (HVLs) defined by structure (see for example, Chothia 1987), or both.

A “biomolecular coating” is a coating on a surface that comprises a molecule that is a naturally occurring biomolecule or biochemical, or a biochemical derived from or based on one or more naturally occurring biomolecules or biochemicals. For example, a biomolecular coating can comprise an extracellular matrix component (e.g., fibronectin, collagens, laminins, other glycoproteins, peptides, glycosaminoglycans, proteoglycans, vitronectin, IntercellularCAMs, VascularCAMs, MAdCAMs), or a derivative thereof, or can comprise a biochemical such as polylysine or polyornithine, which are polymeric molecules based on the naturally occurring biochemicals lysine and ornithine. Polymeric molecules based on naturally occurring biochemicals such as amino acids can use isomers or enantiomers of the naturally-occurring biochemicals. Coatings can also include cell surface receptor or cell surface cognate binding proteins or proteins with affinity for said cell surface proteins.

The term “baseline measurement” refers to a physiologic beginning point for a set of cells to be tested and is based on an evaluation of measurements over a period of time before drug is added. This may include a basal cellular metabolism measurement or CReMS reading prior to exogenous activation. This may alternatively include but not be limited to include the CReMS measurement of a normal healthy cell metabolic function with or without exogenous activation.

The term “Cellular Response Measurement System” or “CReMS” refers to a device that can quantitatively determine a change in a physiological or cellular response parameter in a cell, in and between cells, and between cells and the instrumentation device. In embodiments the cell is a whole label free cell. A change in a physiological or cellular response parameter is measured by determining change in an analyte such as glucose, oxygen, carbon dioxide, amine containing materials such as proteins, amino acids, or of the extracellular matrix, or of a cell signaling molecule, or of cell proliferation, cell morphology, or cytoskeletal rearrangement. An example of a CReMS is a biosensor.

The term “CReMS Signal” as used herein is defined as a measure of cellular physiologic change of cells when those cells are analyzed by a chemo-electric CReMS. The CReMS signal and changes in the CReMS signal can have various units as related to the particular chemo-electric transducer measuring the physiologic change. For example, the CReMS signal may have units of but not be limited to cell index, impedance, wavelength units, pH units, voltage, current, or become dimensionless by using ratios of the units. Any of these units may have a time component. The CReMS signal can be mathematically modified for clarity of interpretation as is frequently done by those practiced in the art of biology, biochemistry and biophysics, for example including normalization, baselining, curve subtracting, or any combination of these. The CReMS signal may be measured at a single time point, or, more preferably, over a continuous series of time points representing a complete pattern of cellular physiologic response.

The term CReM “optical signal” is defined as the wavelength value or change in wavelength value measured as light is reflected from the photonic crystal biosensing CReMS upon which the cells rest. The units are typically in picometers or nanometers though could also become dimensionless if ratios of changes are reported. The “optical signal” could be expressed in said units combined with time. The shift in reflected wavelengths of light is proportional to the mass upon the photonic crystal surface. Thus the “optical signal” is a quantitative measure of the number of cells on the CReMS. Furthermore, the “optical signal” is a measure of the cell physiological status as for example changes in cell morphology, cell adhesion, cell viability, structural rearrangements of the cell lead to differences in the amount of mass upon the sensor that are detected as wavelength shifts.

The term “Cell Index” as used herein is defined as a measurement of impedance and can be applied in one instance of the present invention by measuring at a fixed electrical frequency of, for example, 10 kHz and fixed voltage.

And calculated by the equation Cell Indexᵢ=(Rₜₙ−Rₜ₀)/F

Where:

i=1, 2, or 3 time point

F=15 ohm in one example when the instrument is operated at 10 kHz frequency

Rₜ₀ is the background resistance measured at time point TO.

Rₜₙ is the resistance measured at a time point Tn following cell addition, cell physiologic change, or cell activation.

Cell index is a dimensionless parameter derived as a relative change in measured electrical impedance to represent cell status. When cells are not present or are not well-adhered on the electrodes, the CI is zero. Under the same physiological conditions, when more cells are attached on the electrodes, the CI values are larger. CI is therefore a quantitative measure of cell number present in a well. Additionally, change in a cell physiological status, for example cell morphology, cell adhesion, or cell viability will lead to a change in CI.

The term “measurand” is defined as the quantity intended to be measured in a clinical test. For the invention described herein, the quantity intended to be measured is the change in physiologic response of cells to activation. The change in measurements of a physiologic response of cells to activation can be determined mathematically using a variety of Euclidean mathematical analyses and can be reported numerically in the case of a quantitative test or reported as a positive or negative result in the case of a qualitative test. In both quantitative and qualitative tests, the measurand (e.g. test result) is compared to a cut-off value above which and below which different clinical decisions or interpretations are made.

The term “output value” is a type of measurand and refers to the difference in the CReMS signal, such as measurements of cell adhesion or attachment, that occurs in a viable cell sample from a subject contacted with one or more activator agents that selectively effect signaling pathways and or one or more therapeutic agents that effect the same signaling pathways as the activator agents. The output value may be derived using a variety of Euclidean mathematical analyses of the CReMS signal obtained over a period of time, and can be reported numerically in the case of a quantitative test or reported as a positive or negative result in the case of a qualitative test. For example, a cell sample from a subject contacted with an activator agent (e.g., agonist) alone may generate a CReMS signal of 1,000 units and a cell sample from the same subject contacted with the activator agent and therapeutic agent may generate a CReMs signal of 100 units. The output value in this example would equal 900 CReMS signal units, which is the difference in CReMS signal units measured in the cell sample contacted with activator agent alone and the cell sample contacted with the activator agent and therapeutic agent combined. In both quantitative and qualitative tests, the output value (e.g., test result) may be compared to a cut-off value above which and below which different clinical decisions or interpretations are made.

The term “output value percentage” refers to the percent change in CReMS signal that occurs in a viable cell sample from a patient cell sample contacted with one or more activator agents that selectively effect signaling pathways and one or more therapeutic agents that effect the same signaling pathways as the activator agents, as compared to the sample contacted with one or more activator agents or one or more therapeutic agents alone. For example, a cell sample from a subject contacted with an activator agent alone may generate a CReMS signal of 1,000 units and a cell sample from the same subject contacted with the activator agent and therapeutic agent may generate a CReMs of 100. The output value percentage in this example would equal 90%, which is the difference in CReMS signal units measured in the cell sample contacted with activator agent alone and the cell sample contacted with the activator agent and therapeutic agent combined divided by the CReMS signal units measured in the cell sample contacted with activator agent alone. In both quantitative and qualitative tests, the output value percentage may be compared to a cut-off value percentage above which and below which different clinical decisions or interpretations are made.

The term “basal morphology” refers to the form and structure of a cell or cell sample prior to the introduction of an agent, activator, or stimulus.

The term “cell adhesion” (used interchangeably with “cellular adhesion,” “cell attachment” or “cellular attachment”) refers to the binding of a cell to another cell, to an extracellular matrix component or to a surface (e.g., microtiter plate).

The term “biomarker” refers, in the most general sense, to a biological metric of the condition of a cell or patient health or disease status. A non-limiting listing of general biomarkers includes biologically derived molecules found in a mammal, biological activity of a mammalian cell or tissue, gene copy number, gene mutations, single nucleotide polymorphisms, gene expression levels, mRNA levels, splice variants, transcriptional modifications, post-transcriptional modifications, epi-genetic modifications, cell surface markers, differential expression of a protein or nucleic acid (including all forms of functional RNA), amplification of a nucleic acid, cell morphology, post-translational modifications, protein truncations, phosphorylations, dephosphorylations, ubiquitination, de-ubiquitination, metabolites, hormones at any stage of biosynthesis, cytokines, chemokines, and combinations thereof. A subset of biomarkers are used for diagnostic and therapeutic selection purposes to help pathologists diagnose disease and to help doctors prescribe therapy. Biomarkers typically measure, in fixed, mounted tissue, a gene copy number, a genetic mutation, or the level of a protein without specification of the state or activity of the protein. The present invention includes a new type of biomarker, a physiologic response parameter that is the activity or dynamic result from a live patient cell sample.

The term “biomarker status” refers to assessment of a biomarker(s) in a patient, or patient's cells, and typically is reported as “biomarker positive” when the biomarker is present or “biomarker negative” when the biomarker is absent. When a protein receptor is used as a biomarker, a biomarker positive result is also referred to as the receptor being over-expressed or amplified and a biomarker negative result is referred to as the receptor being normally expressed or non-amplified. For diseases where a biomarker or biomarker signature is a prognostic indicator of disease progression or predicts therapeutic efficacy, current clinical practice relies on the measurement of the amount of biomarker or its related mutations to refine a patient's diagnosis by classifying the patient as either biomarker negative or positive. Determination of biomarker status is often used to guide selection of the drug therapeutic to treat a patient. The cut-off value of a biomarker measurement that is used to distinguish biomarker positive and biomarker negative patients varies from biomarker to biomarker. When the biomarker is a drug target, the cut-off value is the condition above which a patient will receive a therapeutic that targets the biomarker and below which a patient will not receive a therapeutic that targets the biomarker. Clinical trials are typically required to identify the clinical relevance of a biomarker.

The term “biosensor” refers to a device that measures an analyte or a change in an analyte or physiologic condition of a cell. In embodiments, the biosensor typically contains three parts: a biological component or element that binds or recognizes the analyte (including non-limiting examples such as extracellular matrix, cell signaling molecule, or cell proliferation, tissue, cells, metabolites, catabolites, biomolecules, ions, oxygen, carbon dioxide, carbohydrates, proteins etc.), a detector element (operating in a physicochemical manner such as optical, piezoelectric, electrochemical, thermometric, or magnetic), and a transducer associated with both components.

The term “optical biosensor” refers to a device that measures fluorescence, absorption, transmittance, density, refractive index, and reflection of light. In embodiments, an optical biosensor can comprise an optical transducer for converting a molecular recognition or molecular activation event in a living cell, a pathogen, or combinations thereof into a quantifiable signal. Additionally, embodiments could include a photonic crystal device, an optical waveguide device, and a surface plasmon resonance device.

The term “impedance biosensor” refers to a device that measures complex impedance changes (delta Z, or dZ) of live patient cells where impedance (Z) is related to the ratio of voltage to current as described by Ohm's law (Z=V/I). It is sensitive to the local ionic environment at the electrode interface with the cells and detects these changes as a function of voltage and current fluctuations. Physiologic changes of the cells as a result of normal function or activation thereof result in quantifiable changes to the flow of current around the electrodes and influence the magnitude and characteristics of the signal measured. In embodiments, an impedance biosensor can comprise electrodes or an electrical circuit for converting a molecular recognition or molecular activation event in a living cell, a pathogen, or combinations thereof into a quantifiable signal. In embodiments, an ISFET biosensor can comprise an ion selective field effect electrical transducer for converting an analyte recognition or cellular activation event in a living cell, a pathogen, or combinations thereof into a quantifiable signal. When an analyte concentration in an ISFET biosensor changes, the current in the transistor changes, which creates a quantification signal.

The term “cell signaling” refers to the intracellular or intercellular transfer of information. Cells signaling can be achieved by direct contact between cells or by the release of a substance from one cell that is taken up by another cell. Intercellular signaling can occur via an interaction between two molecules (e.g., a ligand and a receptor). Receptor binding can trigger a cascade of intracellular signaling (e.g., initiation of biochemical changes within the cell or modification of the membrane potential).

The term “signaling pathway” refers to a series of cellular components involved in the intracellular or intercellular communication or transfer of information, including cell surface receptors, nuclear receptors, signal regulatory proteins, and intracellular signaling components. As used herein, a particular “signaling pathway” may be named according to the cell surface receptor that triggers the cascade of intracellular signaling or according to any of the components involved in the intracellular signaling. For example, binding of LPA to LPA receptors or S1P to S1P receptors initiates signaling pathway activation that can include PI3K or RTK.

The terms “signaling activity,” “pathway activity,” “cell signaling activity,” and “signaling pathway activity” are used interchangeably and refer to the events occurring during abnormal or normal function of a signaling pathway. Signaling activity is often associated primarily with one cell surface receptor (e.g. a GPCR). However, signaling activity in one pathway may be driven by signaling activity associated with pathway members from other signaling pathways that are upstream, downstream or lateral to a signaling pathways' cell surface receptor. This reflects the interconnected nature of signaling activity where multiple points of pathway convergence, cross-talk between pathways, and feedback loops between pathways can enable signaling activity from one pathway to affect the signaling activity of a different pathway (see Giulliano et al., Bidirectional Crosstalk between the Estrogen Receptor and Human Epidermal Growth Factor Receptor 2 Signaling Pathways in Breast Cancer: Molecular Basis and Clinical Implications, Breast Care (Basel). 2013 August; 8(4): 256-262). Cancer patients whose tumors are driven by signaling activity from two or more interconnected pathways may thus respond to a targeted therapy that binds to a target different than an abnormally functioning pathway's activation point. Due to the nature and complexity of cancer, a cancer patient signaling pathway dysfunction may contain unique interconnections to other pathways that are not commonly described in healthy normal populations.

The term “cytoskeletal organization” refers to the arrangement of the internal scaffold of a cell. A cell's cytoskeleton comprises filaments that serve to support cytoplasmic or membrane elements and/or intracellular organelles. The cytoskeleton also helps to maintain the shape of a cell.

The term “cell proliferation” refers to an increase in the number of cells as a result of cell growth and cell division.

The term “cell survival” refers to the viability of a cell characterized by the capacity to perform certain functions such as metabolism, growth, movement, reproduction, some form of responsiveness, and adaptability.

The term “efficacy” refers to the extent to which a specific intervention produces a beneficial result. In embodiments, the intervention can be a therapeutic agent, such as a small molecule or an antibody or a targeted peptide of an organic reagent with high affinity and specificity for intervention at a known protein. A beneficial result includes without limitation an inhibition of symptoms, a decrease in cell growth, an increase in cell killing, an objective tumor response, an increase in a patient's survival period, an increase in a patient's progression free survival period, an increase in a patient's disease free survival period, a decrease in inflammation, and an increase in immune responsiveness.

The term “extracellular matrix component” refers to a molecule that occurs in the extracellular matrix of an animal. It can be a component of an extracellular matrix from any species and from any tissue type. Non-limiting examples of extracellular matrix components include laminins, collagens, fibronectins, other glycoproteins, peptides, glycosaminoglycans, proteoglycans, etc. Extracellular matrix components can also include growth factors.

The term “global phenotype” refers to a plurality or composite of observable properties of a cell or cell sample as a whole and reflect development, biochemical or physiological properties, phenology, behavior, and products of behavior. A global phenotype may include but not be limited to cell size, cell shape, distinctive protuberances, outgrowths, spreading, attachment foci density, cytoskeletal arrangements, cell proliferation patterns, receptor phagocytosis, or attachment foci number, changes in pH, uptake or efflux of metabolites, signaling proteins and growth factors, oxygen, CO2, glucose, ATP, and ions such as magnesium, calcium, potassium.

The term “event specificity” refers to a physical observation of a specific property of a cell. Such specific properties relate to a specific cellular function, exogenous activation, or pathway agonism/antagonism as part of the intended and/or expected physiological response of the cell to a particular activator or therapeutic agent. Activators and therapeutic agents may be known to be targeted to affect a certain aspect of the cell function such as cytoskeletal structure, or a cellular pathway. The physically observable event is called event specificity because the physically observable event in the cell in the presence of the activator or the therapeutic agent is a reflection of the intended and/or expected effect the activator or therapeutic agent on the cell. For example, the addition of vinblastine to most cell samples on an attachment biosensor type of CReMS produces a profound reduction in signal. Vinblastine is a cellular cytoskeletal scaffolding disrupter. The reduction in signal is a physically observable event of the cell linked specifically to loss of cell shape and attachment caused by the drug action at microtubule molecules.

The term “synergy” or “synergistic” refers to a test result where the CReMS signal, output value, or output value percentage measured when a cell sample is tested with one or more activating agents and/or two or more therapeutic agents is greater than the sum of corresponding measurements obtained when the cell sample is tested with the activator agent[s] and/or therapeutic agents individually. A synergistic result would occur when the simultaneous addition of two therapeutic agents to a cell sample contacted with one or more activating agents produces a greater CReMS signal, output value, or output value percentage than the sum of CReMs signal, output values, or output value percentages obtained when each therapeutic agent is tested individually with the one or more simultaneous activator agents.

The term “Impedance” as used herein is defined by a physical law relating voltage and current by the equation: Impedance (ohm)=Voltage (volts)/Current (amperes) or Z=V/I.

“Mammal” for purposes of treatment or therapy refers to any animal classified as a mammal, including humans, domestic and farm animals, and zoo, sports, or pet animals, such as dogs, horses, cats, cows, and the like. Preferably, the mammal is human.

The term “microcantilever device,” “microcantilever array,” or “microcantilever apparatus” refers to a type of CREMS instrument comprising at least one cantilever, a flexible beam that may be bar-shaped, V-shaped, or have other shapes, depending on its application. One end of the microcantilever is fixed on a supporting base, another end standing freely. Microcantilevers can measure concentrations using electrical methods to detect phase difference signals that can be matched with natural resonant frequencies (examples as described in U.S. Pat. No. 6,041,642, issued Mar. 28, 2000, which is hereby incorporated by reference) Determining a concentration of a target species using a change in resonant properties of a microcantilever on which a known molecule is disposed, for example, a macromolecular biomolecule such as DNA, RNA, or protein. Deflection is measured using optical and piezoelectric methods.

The term “normal functioning” refers to pathways in cells that have a defined system of checks and balances that prevent the cells from becoming dysfunctional from unnatural levels of signaling, replication, loss of contact inhibition, and aberrant gene copying and amplification. In many cases, with pathways beginning in some quiescent or steady basal state, addition of small amounts of activator at the pathway members' EC50 concentration will have only a small transient effect as the cell system recognizes the activator, initiates the pathway activity, and then down regulates the activator effect to maintain balance with other cellular function. Diseased function often is recognizable as over-reaction to an activator, hyper/hypo activity along the pathway, inappropriate inter-pathway activity to accommodate the activator effect, and failure to downregulate the minimal activator effect. Additionally, with some diseased states, a basal state for some pathway members cannot be reached for a pathway. These systems are described as constitutively activated.

The term “normal reference interval” is defined here as the interval between and including two numbers, an upper and lower reference limit, which are estimated to enclose a specified percentage of the values obtained from a population of healthy subjects (e.g. those lacking the disease of interest). For most analytes, the lower and upper reference limits are estimated as the 2.5th and 97.5th percentiles of the distribution of test results for the reference population, respectively. In some cases, only one reference limit is of medical importance, usually an upper limit, say the 97.5th percentile. The confidence intervals for the estimates of the limits of the reference interval can be constructed assuming random sampling of the reference population—generally about 120 reference subjects. The width of each confidence interval depends on the number of reference subjects, as well as the distribution of the observed reference values.

The normal reference range cutoff is determined and set by a process of selection of reference individuals, analytical methods applied to those reference individuals, and concludes with data collection and analysis as defined by the publication Clinical Laboratory Standards Institute Approved Guideline EP28-A3C “Defining, Establishing, and Verifying Reference Intervals in the Clinical Laboratory” whose content is incorporated by reference here in its entirety.

In one embodiment, reference individuals would be individuals free of disease, especially all forms of cancer. A normal reference interval would be determined by testing the normal reference individuals using the methods described herein. The upper limit of the normal reference interval would represent the upper limit of normal pathway activity. In one embodiment, a cut-off value that distinguishes between positive and negative test results would equal the upper limit of the normal reference interval. In other embodiments, the cut-off value would equal the upper limit of the normal reference interval plus any, a combination, all, or a multiple of one, a combination, or all of the following values: limit of detection, limit of blank, limit of quantification, standard deviation of the measurand.

The group of reference individuals free of disease could be further defined by various characteristics beyond the disease of interest. For instance, in an application of the present invention to breast cancer, the group of reference women free of disease may be further defined to include any, combinations, or all of the following characteristics: pre- or post-menopausal, lactating or non-lactating, having borne children, having BRCA gene mutations, presence of diabetes, obesity as determined by BMI (Body Mass Index). abstinence from pharmacologic agents such as hormones or other drug addictions, abstinence from dietary materials such as alcohol and or familial history of cancer.

The terms “abnormal signaling pathway,” “aberrant signaling pathway,” and “dysfunctional signaling pathway” are used interchangeably and refer to a cell signaling pathway that has been disrupted in such a way as to impair the ability of the cell to perform its normal function. The source of the cell signaling disruption and resulting dysfunction is typically a consequence of damage to the genome or proteome that interferes with the signaling pathways' normal function. This damage can be, for example, the result of endogenous processes such as errors in replication of DNA, the intrinsic chemical instability of certain DNA bases, tumor microenvironment, dynamic system adjustment or selection, or from attack by free radicals generated during metabolism. Some inactivating mutations occur in genes responsible for maintaining genomic integrity facilitating the acquisition of additional mutations. Additional mechanisms that affect the genomic level of cellular control involve epigenetic mechanisms whereby the expression of specific genes has been altered by changes to the histone proteins' function. The epigenome function has been demonstrated to be highly adaptive or responsive to many different environmental conditions including conditions that participate in disease etiology and propagation. Various RNA-based mechanisms of pathway dysfunction have been described at the transcriptional, post-transcriptional, translational, and post-translational levels.

Additionally, many actions of pathway dysfunction at the protein level are known to those skilled in the art of cellular molecular biology. Pathway dysfunction can be the result of over or under expression of a pathway member or members or co-factor(s), protein activity present in unnatural cell types or cellular locations, protein interaction with unnatural pathway members also known as pathway cross-reactivity, dysfunctional feedback or feedforward loops, or post-translational modifications. Pathway dysfunction can additionally be the result of activity of the proteome, proteasome, kinome, metabolome, nuclear proteins and factors, cytoplasmic proteins and factors, and or mitochondrial proteins and factors.

When cells with dysfunctional pathways replicate, they can pass on the abnormality to their progeny, which increases the likelihood that the cells become diseased. By analyzing the activity of a cell signaling pathway in live cells, it is possible to determine whether the signaling pathways of the cells are functioning normally or abnormally.

The terms “ultra-sensitive signaling pathway” and “hyperactive signaling pathway” are used interchangeably and refer to a cell signaling pathway in which only a very low level of cellular input difference, such as a signaling pathway activator (e.g., a receptor ligand concentration difference of 1 nM to 10 nM), is capable of causing a low level of activity (e.g., 10% cellular output) to change to a very high level of pathway activation responsiveness (e.g., 90% cellular output). Typically, a component (e.g., enzyme) within an ultra-sensitive or hyperactive signaling pathway is considered to be ultra-sensitive or hyperactive if it requires less than an 81-fold increase in stimulus to drive activity from 10% to 90% maximal response. Signaling pathway ultrasensitivity is described further in and incorporated by reference in their entirety in Ferrell, J. E. and Ha, S. H. (2014) Trends in Biochem. Sci. 39:496-503; Ferrell, J. E. and Ha, S. H. (2014) Trends in Biochem. Sci. 39:556-569; Ferrell, J. E. and Ha, S. H. (2014) Trends in Biochem. Sci. 39:612-618; Huang, C. Y. and Ferrell, J. E. (1996) Proc. Natl. Acad. Sci. USA 93:10078-10083; Kim, S. Y. and Ferrell, J. E. (2007) Cell 128:1133-1145; Trunnell, N. B. et al. (2011) Cell 41:263-274.

The terms “polynucleotide” and “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs and a basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R′, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The term “polypeptide” refers to a peptide or protein containing two or more amino acids linked by peptide bonds, and includes peptides, oligimers, proteins, and the like. Polypeptides can contain natural, modified, or synthetic amino acids. Polypeptides can also be modified naturally, such as by post-translational processing, or chemically, such as amidation, acylation, cross-linking, and the like.

The term “quartz crystal resonators/microbalance” refers to a type of CREMS device that measures mass by measuring the change in frequency of a piezoelectric quartz crystal when it is disturbed by the addition of a small mass such as a virus or any other tiny object intended to be measured. Frequency measurements are easily made to high precision, hence, it is easy to measure small masses.

The term “sample” refers to anything which may contain a moiety to be isolated, manipulated, measured, quantified, detected or analyzed using apparatuses, microplates or methods in the present disclosure. The sample may be a biological sample, such as a biological fluid or a biological tissue. Examples of biological fluids include suspension of cells in a medium such as cell culture medium, urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). The biological samples may further include cell suspensions, solutions containing biological molecules (e.g. proteins, enzymes, nucleic acids, carbohydrates, chemical molecules binding to biological molecules).

The term “cell sample” refers to cells isolated from a particular subject, where the cells are isolated from a subject's biological fluids, excretions, or tissues. Cells isolated from tissue can include tumor cells. Cells isolated from tissue include homogenized tissue, and cellular extracts, and combinations thereof. Cell samples include isolation from, but are not limited to, blood, blood serum, blood plasma, urine, semen, seminal fluid, seminal plasma, prostatic fluid, pre-ejaculatory fluid (Cowper's fluid), excreta, tears, saliva, sweat, biopsy, ascites, cerebrospinal fluid, lymph, marrow, or hair.

The term “CELx” test refers generally to the various embodiments of the methods described herein.

The term “disease cell sample” refers to a plurality of cells from the site of disease or cells that have the characteristic of disease.

The term “healthy cell sample” refers to a cell sample wherein the cells do not have or are extracted from a tissue that does not have the disease that is being tested. For example, when a particular subject is being tested for the effects of a therapeutic agent against the subject's breast cancer, non-cancerous cells or cells from non-breast tissue are considered “healthy.” The term “healthy cell sample” is not a determination or reflection upon the whole health status of the subject. For purposes of deriving a normal reference interval, it is often the case that the healthy cell samples used are obtained from subjects who do not have the disease that is being tested.

The term Analytical “Sensitivity” refers to a test or the detection limit, and is defined as the lowest quantity differentiated from Zero. (e.g. 95% confidence intervals or 2 standard deviations (SD) above the mean of the Zero control are commonly used).

The Term Clinical “Sensitivity” refers to the proportion of subjects with the target condition in whom the test is positive or how often the test is positive when the condition of interest is present. Clinical “Sensitivity” of a test is defined as an estimate of accuracy provided by the calculation: 100%×TP/(TP+FN) where TP is the number of True Positive events for an outcome being tested and FN are the number of False Negatives events, incorrectly determined events as negative.

Clinical “Specificity” refers to the proportion of subjects without the target condition in whom the test is negative or how often the test is negative when the condition of interest is absent. Clinical specificity is estimated by the calculation: 100%×TN/(FP+TN) where TN is the number of True Negative events for an outcome being tested and FP is the number of False Positives, incorrectly determined events as positive.

The term “surface plasmon resonance device” refers to an optical biosensor type of CReMS that measures binding events of biomolecules at a metal surface by detecting changes in the local refractive index.

The term “therapeutic agent” refers to any synthetic or naturally occurring biologically active compound or composition of matter which, when administered to an organism (human or nonhuman animal), induces a desired pharmacologic, immunogenic, and/or physiologic effect by local and/or systemic action. The term encompasses those compounds or chemicals traditionally regarded as drugs, vaccines, and biopharmaceuticals including molecules such as proteins, peptides, hormones, nucleic acids, gene constructs and the like. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment. Therapeutic agents include, but are not limited to, anticancer therapeutics, antipsychotics, anti-inflammatory agents, and antibiotics.

The terms “cytotoxic therapy” and “chemotherapy” refer to treatment with one or more therapeutic agents, wherein the agent(s) exhibits non-specific or non-targeted cytotoxicity against diseased cells (as well as, possibly, non-diseased cells).

The term “targeted therapeutic,” “targeted therapeutic agent,” “targeted pathway drug,” “pathway drug,” or “targeted drug,” refers to any molecule or antibody with therapeutic capacity designed to selectively bind to a specific biomolecule (e.g. protein) involved in a disease process, thereby regulating its activity. Non-limiting examples of biomolecules to which a targeted therapeutic may bind include cell-surface receptors and inter- and intracellular signaling pathway components. As described herein, a “targeted therapeutic” that is administered to a subject for treatment may be the same targeted therapeutic that is tested in vitro as described herein to determine the status of a signaling pathway in a subject cells or, alternatively, the targeted therapeutic that is chosen to be administered for treatment can be a different targeted therapeutic than was tested in vitro but that targets (e.g., selectively affects) the same signaling pathway as the targeted therapeutic that was tested in vitro (e.g., affects the same point or node of the signaling pathway as the tested targeted therapeutic).

“RAS” or “RAS proteins,” as used herein, refer to RAS subfamily members (H-RAS, N-RAS, and K-RAS) of the Ras superfamily of small GTPases. RAS proteins play a vital role in transducing extracellular cues into diverse cellular responses, including cell proliferation, apoptosis, and differentiation. Mutations of RAS constitutively activate downstream effector pathways, which can lead to deregulated cell growth, inhibition of cell death, invasiveness, and induction of angiogenesis. RAS is one of the most common oncogenes in human cancer, with activating mutations being found in about 30% of all human tumors. In addition, many tumors exhibit activation of RAS signaling in the absence of RAS genes (Ji et al. Trends Mol. Med. 18, 27-35). For example, elevated RAS pathway activity is present in more than 50% of human breast tumors—particularly in highly aggressive sub-types, even though they seldom exhibit RAS mutations and are generally considered to be a ‘non-RAS’ tumor type (Sears and Gray, Cancer Discov. 7, 131-133; Siewertsz Van Reesema, Clin. Lab. Int. 40, 18-23)

A “RAS pathway” (also referred to as a “RAS effector pathway” in the art), as used herein, refers to a signaling pathway which transduces signals from activated RAS, receptor tyrosine kinases, and G-protein-coupled receptors (GPCRs) to promote cell proliferation and transformation. Such RAS pathways are known in the art and include, for example, RAF/MEK/ERK, PLCF/PKC, PI3K/AKT/mTOR, RAL-GEF/RAL/TBK1, AKT/BAD/BCL, and TIAM1/RAC/PAK.

A “RAS node,” as used herein, refers to a signaling component (which may include related proteins such as gene isoforms) of a RAS pathway such as RAS/RAF/MEK/ERK, PI3K/AKT/mTOR, RAL/CDC42, RAL-GEF/RAL/TBK1, AKT/BAD/BCL, and TIAM1/RAC/PAK that can be activated by RAS, receptor tyrosine kinases, and G-protein-coupled receptors. For instance, heterodimers of the ERBB family can activate the PI3K/AKT/mTOR cascade directly through sites on HER3 or adaptors such as GRB2 while also directly activating the RAS/RAF/MEK/ERK cascade through sites on EGFR/HER2/HER4 or adaptors such as SHC. Alternatively, GPCRs such as SIP and LPA can initiate hyperactivated signaling through the RAF/MEK/ERK, PI3K/AKT/mTOR, and RAL/CDC42 pathways. An “inhibitor of a RAS node,” as used herein, refers to an inhibitor of a component (which may include related proteins such as gene isoforms) within a given RAS pathway which transduces a signal from activated RAS, a receptor tyrosine kinase or a GPCR. RAS nodes are well known in the art and include, for example, PI3K, AKT (Akt1, Akt2, Akt3), mTOR, S6K, RAL (RalA, RalB), RalBP1, RalGDS, TBK1, PLCε, RGL (Rg11, Rg12, Rg13), RAF (A-RAF, B-RAF, C-RAF), MEK (MEK1/2), ERK (ERK1/2), TIAM1/2, RAC, PAK, Bcl-xL, and BCL-2 (see, e.g., Clark, et al. Journal of Cell Science (2020) 133, jcs238865; Baines et al., Future Med Chem 2011; 3:1787-808; Vigil et al., Nat Rev Cancer 2010; 10:842-57; Moghadam et al., Cancer Med. 2017; 6:2998-3013; Kang et al., Front Oncol. 2012:2:206; Carne Trecesson et al., Nature Communications 2017; 8:1123; Gaspar et al., Small GTPases 2020,

DOI:10.1080/21541248.2020.1724596). In some embodiments, the inhibitor of a RAS node inhibits one or more RAS nodes selected from the group consisting of: PI3K, AKT, mTOR, RAF, MEK, ERK, and BCL. Gedatolisib is an example of an inhibitor which inhibits more than one RAS node, i.e., PI3K and mTOR. In some embodiments, the RAS pathway and RAS node promote cell proliferation and/or cell survival.

The term “PI3K” refers to the phosphatidylinositol-3-kinase family of lipid kinases. PI3Ks have sequence homology in their kinase domains, but have distinct substrate specificities, expression patterns, and modes of regulation. PI3Ks are subdivided into four classes (Classes I-IV). Classes I, II, and III are lipid kinases that phosphorylate phosphoinositide lipids. Class I PI3Ks include four isoforms: p110α (PI3Kα), p110β (PI3Kβ), p110δ (PI3Kδ), and p110γ (PI3Kγ), and are further subdivided into IA or IB according to their regulatory subunit. Class IA, which includes PI3Kα, PI3K3, and PI3Kδ, has a p85 regulatory subunit containing two SH2 domains which is activated by interaction with RTKs. Class IB, which includes PI3K7, has a p101 regulatory subunit which is activated by GPCRs. Class II consists of four members (CIIα, CIIβ, and CIIδ) and Class III consists of one member, Vps34. Class IV kinases, also call PIKKs (PI3K-related protein kinase) are a subfamily of serine/threonine protein kinases that share homology with PI3K, but phosphorylate proteins rather than phosphoinositide lipids. PIKKs include mTOR (mammalian target of rapamycin).

The term “AKT” or “Protein kinase B (PKB)” refers to a serine/threonine kinase that regulates cell survival signals in response to growth factors, cytokines, and oncogenic RAS. AKT is recruited to the plasma membrane of cells through interactions with the secondary messenger PIP3, which is formed by phosphorylation of lipids on the plasma membrane by PI3K, and subsequently mediates downstream responses, including proliferation, migration, angiogenesis, and survival.

The term “mTOR” refers to the protein mammalian target of rapamycin, which is a serine/threonine kinase related to the PI3K family and is a downstream effector of the PI3K/AKT signaling pathway. mTOR functions as a regulator of cell growth and metabolism, and exists in two complexes, mTORC1 and mTORC2.

The term “RAF” refers to a protein encoded by a gene family consisting of three genes, yielding three isoforms which share highly conserved amino-terminal regulatory regions and catalytic domains at the carboxy terminus: A-RAF, B-RAF, and C-RAF (RAF1). B-RAF is recruited by Ras:GTP to the intracellular cell membrane where B-RAF becomes activated. In turn, B-RAF activates MEK1/2, and MEK1/2 activate ERK1/2. Mutations in B-RAF (e.g., V600E), which is found in a significant fraction of malignancies, allow for B-RAF to signal independently of upstream signals and promote excessive activation of downstream MEK and ERK signaling, which leads to excessive cell proliferation and survival and oncogenesis. Overactivation of downstream signaling by mutated B-RAF is implicated in many malignancies. Mutated B-RAF is found in a significant fraction of malignancies.

The term “MEK” or “mitogen-activated protein kinase kinase” is a kinase which phosphorylates mitogen-activated protein kinase (MAPK). Subtypes of MEK include MAP2K1 (MEK1), MAP2K2 (MEK2), MAP2K3 (MKK3), MAP2K4 (MKK4), MAP2K5 (MKK5), MAP2K6 (MKK6), MAP2K7 (MKK7). As used herein, “MEK” preferably refers to MEK1 or MEK2, or both MEK1 and MEK2.

The term “ERK” or “extracellular signal-regulated kinase” refers to a serine/threonine kinase which is member of the MAPK family. ERK is a component of the RAS/RAF/MEK signaling pathway, wherein RAF activates MEK1/2, and MEK1/2 activates ERK1/2. As used herein, “ERK” can refer to ERK1 or ERK2, or both ERK1 and ERK2.

The term “Bcl-2,” which is encoded by the BCL2 gene, refers to a protein encoded by the B-Cell CLL/Lymphoma 2 gene, which is located on chromosome 18. Bcl-2 is a proto-oncogene which regulates mitochondrial permeabilization and is a key point in the intrinsic cell apoptosis pathway. It is also the founding member of the Bcl-2 family of cell death regulators, anti-apoptotic members of which include, e.g., Bcl-2, Bcl-xL, Bcl-w, Mcl-1, and Al. “BCL,” as used herein, refers to the anti-apoptotic members of the Bcl-2 family. “Bcl-xL” or “B-cell lymphoma-extra large)” is a member of the Bcl-2 family and refers to the long form of Bcl-x generated by alternative splicing in exon 2 of Bcl-x. Bcl-xL is an anti-apoptotic protein that prevents apoptosis in part by inhibiting Bax.

The term “receptor tyrosine kinase” or “RTK” refers to a member of the growth factor receptor family of proteins. Growth factor receptors are typically involved in cellular processes including cell growth, cell division, differentiation, metabolism and cell migration. RTKs have a conserved domain structure including an extracellular domain, a membrane-spanning (transmembrane) domain, and an intracellular tyrosine kinase domain. Classification by structural motifs has identified 20 families of RTKs, each with a conserved tyrosine kinase domain. Examples of RTKs include erythropoietin-producing hepatocellular (EPH) receptors, epidermal growth factor (EGF) receptors, fibroblast growth factor (FGF) receptors, platelet-derived growth factor (PDGF) receptors, vascular endothelial growth factor (VEGF) receptor, cell adhesion RTKs (CAKs), Tie/Tek receptors, insulin-like growth factor (IGF) receptors, and insulin receptor related (IRR) receptors. Exemplary genes encoding RTKs include ERBB2 (also known as HER2), ERBB3, DDR1, DDR2, TKT, EGFR, EPHA1, EPHAB, FGFR2, FGFR4, FLT1 (also known as VEGFR-1), FLK1 (also known as VEGFR-2) MET, PDGFRA, PDGFRB, and TEK.

The term “G-protein coupled receptor” or “GPCR” refers to a membrane-associated receptor that associates with a G-protein and has 7 alpha helical transmembrane domains. GPCRs associate with a ligand or agonist and also associate with and activate G-proteins. “GPCR activity” refers to the ability of a GPCR to transduce a signal.

The term “lysophospholipid GPCR” refers to a class of GPCRs that are activated by lysophospholipids, such as lysophosphatidic acid (LPA), sphingosine 1-phosphate (S1P), lysophosphatidyl inositol (LPI), and a lysophosphatidylserine (LyPS).

The term “agonist” refers to a compound which binds to a receptor and causes a response in a cell. For example, an “agonist of GPCR signaling” or “GPCR agonist” refers to an agent (e.g., a ligand) that activates the GPCR upon binding to the GPCR and induces an intracellular response mediated by the GPCR. An exemplary GPCR agonist is a lysophospholipid such as LPA or S1P which binds to a lysophospholipid GPCR, such as an LPA receptor or S1P receptor. Additional examples of GPCR agonists are described herein.

The term “RAS node therapy” or “RAS node-targeted therapy” refers to treatments using one or more therapeutic agents that are designed to specifically target a RAS node within a RAS pathway (e.g., PI3K, AKT, mTOR, RAF, MEK, ERK, and BCL), including but not limited to antibodies and small molecules which target the RAS node.

The term “PI3K therapy” or “PI3K-targeted therapy” refers to treatments using one or more therapeutic agents that are designed to specifically target the PI3K molecule and/or signaling pathway(s), including but not limited to, for example antibodies and small molecules that target the PI3K molecule and/or signaling pathway(s).

The term “RTK therapy” or “RTK-targeted therapy” refers to treatments using one or more therapeutic agents that are designed to specifically target the RTK molecule and/or signaling pathway(s), including but not limited to, for example antibodies and small molecules that target the RTK molecule and/or signaling pathway(s).

The term “anti-proliferative drug,” “anti-proliferative agent,” or “apoptosis inducing drug” refers to any molecule or antibody with therapeutic capacity that functions to reduce cell division, reduce cell growth, or kill cells. In many cases, the activity of these drugs is directed towards broad classes of biomolecules (e.g. DNA intercalation) involved in normal cellular processes and thus the drug may be less discriminant towards cell disease status.

The term “therapeutically active” refers to an effect on a signaling pathway that occurs when a subject's cancer cells are contacted with a targeted therapeutic agent, such as a small molecule or an antibody or a targeted peptide or any organic reagent with high affinity and specificity for intervention at a known protein. A therapeutically active targeted therapeutic agent is one that affects the signaling pathway(s) the targeted therapeutic agent is intended to affect. A targeted therapeutic agent that is more therapeutically active in a subject's cancer cells than another targeted therapeutic agent is one that has a greater effect on the signaling pathway it is intended to affect than the other therapeutic agent has on the signaling pathway the other therapeutic agent is intended to affect. There is evidence that in at least certain cancers, two or more signaling pathways may be interconnected with multiple points of convergence, cross-talk and feedback loops, such that inhibiting only one of the pathways can still result in the maintenance of signaling via the other (reciprocal pathway) (see e.g., Saini, K. S. et al. (2013) Cancer Treat. Rev. 39:935-946). Since the activities of one signaling pathway can affect the activities of other signaling pathways, a targeted therapeutic may disrupt signaling activity associated indirectly with that targeted therapeutic's binding site. In these cases, a targeted therapy may be considered therapeutically active if it is found to inhibit signaling activities for pathways not directly associated with that targeted therapeutic binding site.

A “variant” of a polypeptide refers to a polypeptide that contains an amino acid sequence that differs from a reference sequence. The reference sequence can be a full-length native polypeptide sequence or any other fragment of a full-length polypeptide sequence. In some embodiments, the reference sequence is a variable domain heavy chain or variable domain light chain consensus sequence. A polypeptide variant generally has at least about 80% amino acid sequence identity with the reference sequence.

### Methods of Selection and Treatment of Cancer Patients

Provided herein is a method of selecting a human cancer patient for treatment with a RAS node (e.g., PI3K, AKT, mTOR, RAF, MEK, ERK, and BCL) or RTK targeted therapeutic agent. The method used to select the human subject for treatment involves evaluating the effects of an inhibitor of a RAS node or RTK on GPCR-mediated signaling in the subject's cancer cells. The methods used to select the human cancer patient for treatment with a RAS node or RTK targeted therapeutic agent involve evaluating the amount of activity initiated in a patient's cells by a GPCR agonist (e.g., a lysophospholipid such as LPA or S1P) and inhibited by an inhibitor of a RAS node or RTK (e.g., a RAS node or RTK targeted therapeutic agent). Thus, the methods described herein allow for, e.g., identifying and selecting a patient with abnormal GPCR signaling involving the RAS node or RTK for treatment with a RAS node or RTK targeted therapeutic agent.

Accordingly, in one aspect, provided herein is a method of selecting a human subject diagnosed with cancer for treatment with a RAS node targeted therapeutic, the method comprising:

culturing a sample of viable cancer cells obtained from the subject;

contacting (1) a first portion of the sample with an agonist of G-protein coupled receptor (GPCR) signaling and an inhibitor of a RAS node, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

selecting the subject for treatment with a RAS node targeted therapeutic which inhibits the same RAS node as the inhibitor if the output value that characterizes the change in cell adhesion or attachment is equal to or greater than a pre-determined cut-off value.

In another aspect, provided herein is a method of treating a human subject diagnosed with cancer mediated by a RAS node, wherein cancer cells of the subject have been tested in a method comprising:

culturing a sample of viable cancer cells obtained from the subject;

contacting (1) a first portion of the sample with an agonist of GPCR signaling and an inhibitor of a RAS node, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

administering to the subject a RAS node targeted therapeutic which inhibits the same RAS node as the inhibitor if the output value that characterizes the change in cell adhesion or attachment is equal to or greater than a pre-determined cut-off value.

In certain embodiments, rather than using a pre-determined cut-off value, the decision to select a patient or treat a patient with a RAS node targeted therapeutic is based on an output value, expressed as a percentage (hereafter, “output value percentage”), that characterizes whether a change in cell adhesion or attachment has occurred in first portion as compared to the second portion. For instance, an output value percentage of 30% would be assigned if the readout for the second portion (i.e., agonist alone) is 1000 and the readout for the first portion (agonist+inhibitor) is 700. The difference between the two output values, 300, represents the amount of agonist activity that is reduced by the inhibitor. Similarly, an output value percentage of 75% would be assigned if the readout for the second portion is 1000 (i.e. agonist alone) and the readout for the first portion (agonist+inhibitor) is 250.

Accordingly, in another aspect, provided herein is a method of selecting a human subject diagnosed with cancer for treatment with a RAS node targeted therapeutic, the method comprising:

culturing a sample of viable cancer cells obtained from the subject;

contacting (1) a first portion of the sample with an agonist of GPCR signaling and an inhibitor of a RAS node, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with a RAS node targeted therapeutic which inhibits the same RAS node as the inhibitor if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In another aspect, provided herein is a method of treating a human subject diagnosed with cancer mediated by a RAS node, wherein cancer cells of the subject have been tested in a method comprising:

culturing a sample of viable cancer cells obtained from the subject;

contacting (1) a first portion of the sample with an agonist of GPCR signaling and an inhibitor of a RAS node, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject a RAS node targeted therapeutic which affects the same RAS node as the inhibitor if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

Exemplary RAS nodes which can be inhibited by the inhibitor include, but are not limited to, PI3K, AKT (Akt1, Akt2, Akt3), mTOR, S6K, RAL (RalA, RalB), RalBP1, RalGDS, TBK1, PLCF, RGL (Rg11, Rg12, Rg13), RAF (A-RAF, B-RAF, C-RAF), MEK (MEK1/2), ERK (ERK1/2), TIAM1/2, RAC, PAK, Bcl-xL, and Bcl-2, and a combination thereof. In some embodiments, the RAS node inhibited by the inhibitor is selected from the group consisting of PI3K, AKT, mTOR, RAF, MEK, ERK, Bcl-xL, and a combination thereof.

In some embodiments, the inhibitor of the RAS node inhibits a RAS node selected from the group consisting of PI3K, AKT (Akt1, Akt2, Akt3), mTOR, S6K, RAL (RalA, RalB), RalBP1, RalGDS, TBK1, PLCF, RGL (Rg11, Rg12, Rg13), RAF (A-RAF, B-RAF, C-RAF), MEK (MEK1/2), ERK (ERK1/2), TIAM1/2, RAC, PAK, Bcl-xL, and Bcl-2, and a combination thereof. In some embodiments, the inhibitor of the RAS node inhibits a RAS node selected from the group consisting of PI3K, AKT, mTOR, RAF, MEK, ERK, Bcl-xL, and a combination thereof.

In some embodiments, the first portion of the sample is contacted with two or more inhibitors of RAS nodes, each of which inhibits a different RAS node. For example, the two or more inhibitors may inhibit a combination of RAS nodes selected from the group consisting of: (a) PI3K, mTOR, and BCL (e.g., Bcl-xL, Bcl-2, Mcl-1), (b) PI3K, mTOR, and RAF, (c) PI3K, mTOR, and ERK, and (d) PI3K, mTOR, and MEK. Other combinations of two or more RAS nodes are also contemplated and within the scope of the methods described herein. In some embodiments, the subject is treated or selected for treatment with a combination of RAS node targeted therapeutics which inhibit the different RAS nodes.

In some embodiments, the inhibitor of the RAS node inhibits more than one RAS node. For example, the inhibitor may be a multi-specific inhibitor of two or more RAS nodes. By way of example, the inhibitor gedatolisib inhibits the RAS nodes mTOR and PI3K.

In some embodiments, the RAS node targeted in the methods described herein is PI3K, and the inhibitor of the RAS node is a PI3K inhibitor.

In some embodiments, the PI3K inhibitor selectively inhibits the same PI3K isoform. For example, in one embodiment, the PI3K inhibitor selectively inhibits the p110α catalytic subunit of PI3K. In another embodiment, the PI3K inhibitor selectively inhibits the p1100 catalytic subunit of PI3K. In another embodiment, the PI3K inhibitor selectively inhibits the p110γ catalytic subunit of PI3K. In another embodiment, the PI3K inhibitor selectively inhibits the p110δ subunit of PI3K. Isoform-selective PI3K inhibitors are well known in the art. In some embodiments, the PI3K inhibitor selectively inhibits class IA PI3Ks (i.e., p110α, p110β, and p110γ).

In some embodiments, the PI3K inhibitor inhibits more than one isoform of the p110 subunit of PI3K. In some embodiments, the PI3K inhibitor inhibits all isoforms of the p110 catalytic subunit of PI3K.

Non-limiting examples of PI3K inhibitors include, e.g., wortmannin, LY294002, hibiscone C, Idelalisib (GS-1101, CAL-101), Copanlisib, Duvelisib, Alpelisib, (BYL719), Taselisib (GDC-0032), GDC-0077, Perifosine, Idealisib, Pilaralisib (XL147), Buparlisib (BKM120), Duvelisib, Umbralisib, PX-866, Dactolisib, CUDC-907, Voxtalisib, ME-401, IPI-549, SF1126, RP6530, INK1117, Pictilisib (GDC-0941), Palomid 529, SAR260301, GSK1059615, GSK2636771, CH5132799, CZC24832, AZD6482, AZD8835, WX-037, AZD8186, KA2237, CAL-120, AMG-319, AMG-511, HS-173, INCB050465, INCB040093, TGR-1202, ZSTK474, PWT33597, IC87114, TG100-115, TGX221, CAL263, RP6530, PI-103, GNE-477, IPI-145, BAY 80-6946, BAY1082439, PX866, BEZ235, MKM120, MLN1117, SAR245408, and AEZS-136, serabelisib (TAK-117), gedatolisib, omipalisib, and pilarlisib.

In some embodiments, the PI3K inhibitor also inhibits mTOR signaling. Such inhibitors include, but are not limited to, BGT-226, DS-7423, PF-04691502, PKI-179, GSK458V, GSK2126458, P7170, SB2343, VS-5584, NVP-BEZ235, SAR245409, GDC-0084, GDC-0980, LY3023414, PQR309, XL765, SF-1126, PF-05212384, PKI-587, dactolisib, pictilisib (GDC-0941), LY3023414, gedatolisib, omipalisib, and PQR309.

In some embodiments, the PI3K inhibitor also inhibits RAF signaling. Such inhibitors include, but are not limited to, ASN003.

In some embodiments, the PI3K inhibitor also inhibits HDAC signaling. Such inhibitors include, but are not limited to, fimepinostat.

In one embodiment, the PI3K inhibitor and/or PI3K targeted therapeutic is alpelisib. In another embodiment, the PI3K inhibitor and/or PI3K targeted therapeutic is IPI-549.

In one embodiment, provided herein is a method of selecting a human subject diagnosed with cancer for treatment with a PI3K targeted therapeutic, the method comprising:

culturing a sample of viable cancer cells obtained from the subject;

contacting (1) a first portion of the sample with an agonist of GPCR signaling and an inhibitor of PI3K signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with a PI3K targeted therapeutic which inhibits the same PI3K signaling pathway as the inhibitor if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In another embodiment, provided herein is a method of treating a human subject diagnosed with cancer mediated by PI3K signaling, wherein cancer cells of the subject have been tested in a method comprising:

culturing a sample of viable cancer cells obtained from the subject;

contacting (1) a first portion of the sample with an agonist of GPCR signaling and an inhibitor of PI3K signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject a PI3K targeted therapeutic which affects the same PI3K signaling pathway if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In some embodiments, the RAS node targeted in the methods described herein is ERK, and the inhibitor of the RAS node is an ERK inhibitor. In some embodiments, the ERK inhibitor selectively inhibits ERK1. In other embodiments, the ERK inhibitor selectively inhibits ERK2. In yet other embodiments, the ERK inhibitor inhibits both ERK1 and ERK2. ERK inhibitors, including isoform-specific ERK inhibitors, are well known in the art and include, for example, ravoxertinib, SCH772984, SCH900353 (MK8353), ulixertinib, AZD0364 (ATG017), VX-11e (VTX-113), CC-90003, LY3214996, FR180204, ASN007, E6201, and GDC0994.

In one embodiment, provided herein is a method of selecting a human subject diagnosed with cancer for treatment with an ERK targeted therapeutic, the method comprising:

culturing a sample of viable cancer cells obtained from the subject;

contacting (1) a first portion of the sample with an agonist of GPCR signaling and an inhibitor of ERK signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with an ERK targeted therapeutic which inhibits the same ERK signaling pathway as the inhibitor if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In another embodiment, provided herein is a method of treating a human subject diagnosed with cancer mediated by ERK signaling, wherein cancer cells of the subject have been tested in a method comprising:

culturing a sample of viable cancer cells obtained from the subject;

contacting (1) a first portion of the sample with an agonist of GPCR signaling and an inhibitor of ERK signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject an ERK targeted therapeutic which affects the same ERK signaling pathway if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In some embodiments, the RAS node targeted in the methods described herein is MEK, and the inhibitor of the RAS node is a MEK inhibitor. In some embodiments, the MEK inhibitor selectively inhibits MEK1. In other embodiments, the MEK inhibitor selectively inhibits MEK2. In yet other embodiments, the MEK inhibitor inhibits both MEK1 and MEK2. MEK inhibitors, including isoform-specific MEK inhibitors, are well known in the art and include, for example, trametinib, binimetinib, pimasertib, cobimetinib, PD901, U0126, selumetinib, PD325901, TAK733, CI-1040 (PD184352), PD198306, PD334581, PD98059, and SL327.

In one embodiment, provided herein is a method of selecting a human subject diagnosed with cancer for treatment with a MEK targeted therapeutic, the method comprising:

culturing a sample of viable cancer cells obtained from the subject;

contacting (1) a first portion of the sample with an agonist of GPCR signaling and an inhibitor of MEK signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with a MEK targeted therapeutic which inhibits the same MEK signaling pathway as the inhibitor if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In another embodiment, provided herein is a method of treating a human subject diagnosed with cancer mediated by MEK signaling, wherein cancer cells of the subject have been tested in a method comprising:

culturing a sample of viable cancer cells obtained from the subject;

contacting (1) a first portion of the sample with an agonist of GPCR signaling and an inhibitor of MEK signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject a MEK targeted therapeutic which affects the same MEK signaling pathway if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In some embodiments, the RAS node targeted in the methods described herein is AKT, and the inhibitor of the RAS node is an AKT inhibitor. In some embodiments, the AKT inhibitor selectively inhibits AKT 1. In other embodiments, the AKT inhibitor selectively inhibits AKT 2. In yet other embodiments, the AKT inhibitor inhibits both AKT 1 and AKT 2. AKT inhibitors, including isoform-specific AKT inhibitors, are well known in the art and include, for example, ARQ 751, borussertib, TAS-117, afuresertib, M2698, MK-2206, capivasertib, and ipatasertib.

In one embodiment, provided herein is a method of selecting a human subject diagnosed with cancer for treatment with a AKT targeted therapeutic, the method comprising:

culturing a sample of viable cancer cells obtained from the subject;

contacting (1) a first portion of the sample with an agonist of GPCR signaling and an inhibitor of AKT signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with a AKT targeted therapeutic which inhibits the same AKT signaling pathway as the inhibitor if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In another embodiment, provided herein is a method of treating a human subject diagnosed with cancer mediated by AKT signaling, wherein cancer cells of the subject have been tested in a method comprising:

culturing a sample of viable cancer cells obtained from the subject;

contacting (1) a first portion of the sample with an agonist of GPCR signaling and an inhibitor of AKT signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject a AKT targeted therapeutic which affects the same MEK signaling pathway if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In some embodiments, the RAS node targeted in the methods described herein is mTOR, and the inhibitor of the RAS node is an mTOR inhibitor. In some embodiments, the mTOR inhibitors also inhibit other RAS nodes, such as PI3K (e.g., dual PI3K/mTOR inhibitors). In one embodiment, the mTOR inhibitor inhibits mTORC1. In another embodiment, the mTOR inhibitor inhibits mTORC2. In yet another embodiment, the mTOR inhibitor inhibits both mTORC1 and mTORC2. mTOR inhibitors are well known in the art and include, for example, gedatolisib, sirolimus, everolimus, temsirolimus, dactolisib, AZD8055, ABTL-0812, PQR620, GNE-493, KU0063794, torkinib, ridaforolimus, sapanisertib, voxtalisib, torin 1, torin 2, OSI-027, PF-04691502, apitolisib, GSK1059615, WYE-354, vistusertib, WYE-125132, BGT226, palomid 529, WYE-687, WAY600, GDC-0349, XL388, bimiralisib (PQR309), omipalisib (GSK2126458, GSK458), onatasertib (CC-223), samotolisib, omipalisib, RMC-5552, and GNE-477

In one embodiment, provided herein is a method of selecting a human subject diagnosed with cancer for treatment with an mTOR targeted therapeutic, the method comprising:

culturing a sample of viable cancer cells obtained from the subject;

contacting (1) a first portion of the sample with an agonist of GPCR signaling and an inhibitor of mTOR signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with an mTOR targeted therapeutic which inhibits the same mTOR signaling pathway as the inhibitor if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In another embodiment, provided herein is a method of treating a human subject diagnosed with cancer mediated by mTOR signaling, wherein cancer cells of the subject have been tested in a method comprising:

culturing a sample of viable cancer cells obtained from the subject;

contacting (1) a first portion of the sample with an agonist of GPCR signaling and an inhibitor of mTOR signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject an mTOR targeted therapeutic which affects the same mTOR signaling pathway if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In some embodiments, the RAS node targeted in the methods described herein is RAF, and the inhibitor of the RAS node is a RAF inhibitor. In one embodiment, the RAF inhibitor selectively inhibits A-RAF. In another embodiment, the RAF inhibitor selectively inhibits B-RAF. In yet another embodiment, the RAF inhibitor selectively inhibits C-RAF. In a further embodiment, the RAF inhibitor inhibits more than one isoform of RAF. In yet a further embodiment, the RAF inhibit inhibits all isoforms of RAF. RAF inhibitors are well known in the art and include, for example, PLX7904, GDC-0879, belvarafenib (GDC-5573), SB590885, encorafenib, RAF265, RAF709, dabrafenib (GSK2118436), TAK-632, TAK580, PLX-4720, CEP-32796, sorafenib, vemurafenib (PLX-4032), AZ-628, GW5074, ZM-336372, NVP-BHG712, CEP32496, PLX4032, PF-0728489, and LGX-818.

In one embodiment, provided herein is a method of selecting a human subject diagnosed with cancer for treatment with a RAF targeted therapeutic, the method comprising:

culturing a sample of viable cancer cells obtained from the subject;

contacting (1) a first portion of the sample with an agonist of GPCR signaling and an inhibitor of RAF signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with a RAF targeted therapeutic which inhibits the same RAF signaling pathway as the inhibitor if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In another embodiment, provided herein is a method of treating a human subject diagnosed with cancer mediated by RAF signaling, wherein cancer cells of the subject have been tested in a method comprising:

culturing a sample of viable cancer cells obtained from the subject;

contacting (1) a first portion of the sample with an agonist of GPCR signaling and an inhibitor of RAF signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject a RAF targeted therapeutic which affects the same RAF signaling pathway if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In some embodiments, the RAS node targeted in the methods described herein is Bcl-xL, and the inhibitor of the RAS node is a Bcl-xL inhibitor. Bcl-xL inhibitors are well known in the art and include, for example, navitoclax (ABT-263), G139, GDC-0199 (ABT-199, venetoclax), sabutoxlax (B1-97C1), ABT-737, AT101, TW037, A1331852, BXI-61, WEHI-539, A1331852, and BXI-72. In some embodiments, the Bcl-xL inhibitor further inhibits one or more members of the Bcl-2 family, such as Bcl-2.

In one embodiment, provided herein is a method of selecting a human subject diagnosed with cancer for treatment with a Bcl-xL targeted therapeutic, the method comprising:

culturing a sample of viable cancer cells obtained from the subject;

contacting (1) a first portion of the sample with an agonist of GPCR signaling and an inhibitor of Bcl-xL signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with a Bcl-xL targeted therapeutic which inhibits the same Bcl-xL signaling pathway as the inhibitor if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In another embodiment, provided herein is a method of treating a human subject diagnosed with cancer mediated by Bcl-xL signaling, wherein cancer cells of the subject have been tested in a method comprising:

culturing a sample of viable cancer cells obtained from the subject;

contacting (1) a first portion of the sample with an agonist of GPCR signaling and an inhibitor of Bcl-xL signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject a Bcl-xL targeted therapeutic which affects the same Bcl-xL signaling pathway if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In some embodiments, the RAS node targeted in the methods described herein is Bcl-2, and the inhibitor of the RAS node is a Bcl-2 inhibitor. Bcl-2 inhibitors are well known in the art and include, for example, navitoclax (ABT-263), Obatoclax Mesylate (GX15-070), AZD5991, Jaceosidin, A-1210477, ML311, Gambogic Acid, S63845, Marinopyrrole A (Maritoclax), VU661013, UMI-77, and S64315 (MIK665). In some embodiments, the Bcl-2 inhibitor (e.g., navitoclax) further inhibits one or more members of the Bcl-2 family, such as Bcl-xL.

In one embodiment, provided herein is a method of selecting a human subject diagnosed with cancer for treatment with a Bcl-2 targeted therapeutic, the method comprising:

culturing a sample of viable cancer cells obtained from the subject;

contacting (1) a first portion of the sample with an agonist of GPCR signaling and an inhibitor of Bcl-2 signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with a Bcl-2 targeted therapeutic which inhibits the same Bcl-2 signaling pathway as the inhibitor if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

In another embodiment, provided herein is a method of treating a human subject diagnosed with cancer mediated by Bcl-2 signaling, wherein cancer cells of the subject have been tested in a method comprising:

culturing a sample of viable cancer cells obtained from the subject;

contacting (1) a first portion of the sample with an agonist of GPCR signaling and an inhibitor of Bcl-2 signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject a Bcl-2 targeted therapeutic which affects the same Bcl-2 signaling pathway if the output value that characterizes the change in cell adhesion or attachment is equal to or greater than a pre-determined cut-off value, or if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or for example equal to or greater than 95%.

Once an inhibitor of a RAS node (e.g., PI3K inhibitor, AKT inhibitor, ERK inhibitor, MEK inhibitor, mTOR inhibitor, RAF inhibitor, Bcl-xL inhibitor), or inhibitors of RAS nodes, is determined to affect GPCR signaling in a patient's cancer cells, e.g., as reflected in an output value that is equal to or greater than a pre-determined cut-off or an output value percentage that is equal to or greater than 30%, for example, equal to or greater than 50%, the cancer patient is selected for treatment with a RAS node targeted therapeutic (e.g., PI3K targeted therapeutic, AKT targeted therapeutic, ERK targeted therapeutic, MEK targeted therapeutic, mTOR targeted therapeutic, RAF targeted therapeutic, Bcl-xL targeted therapeutic), or RAS node targeted therapeutics. In some embodiments, the inhibitor of the RAS node and the RAS node targeted therapeutic are the same compound. Alternatively, once GPCR signaling in the patient's cancer cells have been determined to be responsive to the inhibition of a RAS node, the RAS node targeted therapeutic used to treat the patient need not be the same compound as the inhibitor, so long as RAS node targeted therapeutic inhibits the same RAS node as the inhibitor. Accordingly, in other embodiments, the inhibitor of the RAS node and RAS node targeted therapeutic are different compounds. By way of example, when targeting the RAS node PI3K, the PI3K inhibitor used to determine whether GPCR signaling in the patient's cancer cells is responsive to the inhibition of PI3K may be LY294002, whereas the PI3K targeted therapeutic administered to the patient if the patient's cancer cells are responsive to LY294002 may be the PI3K inhibitor alpelisib.

The methods described herein may also be used to target signaling nodes co-involved with RAS, such as RTKs. Accordingly, in another aspect, provided herein is a method of selecting a human subject diagnosed with cancer for treatment with an RTK targeted therapeutic, the method comprising:

culturing a sample of viable cancer cells obtained from the subject;

contacting (1) a first portion of the sample with an agonist of GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

selecting the subject for treatment with an RTK targeted therapeutic if the output value that characterizes the change in cell adhesion or attachment is equal to or greater than a pre-determined cut-off value.

In another aspect, provided herein is a method of treating a human subject diagnosed with cancer mediated by RTK signaling, wherein cancer cells of the subject have been tested in a method comprising:

culturing a sample of viable cancer cells obtained from the subject;

contacting (1) a first portion of the sample with an agonist of GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

administering to the subject a targeted therapeutic that inhibits RTK signaling if the output value that characterizes the change in cell adhesion or attachment is equal to or greater than a pre-determined cut-off value.

In certain embodiments, rather than using a pre-determined cut-off value, the decision to select a patient or treat a patient with an RTK targeted therapeutic is based on an output value percentage that characterizes whether a change in cell adhesion or attachment has occurred in first portion as compared to the second portion.

Accordingly, in another aspect, provided herein is a method of selecting a human subject diagnosed with cancer for treatment with a RTK targeted therapeutic, the method comprising:

culturing a sample of viable cancer cells obtained from the subject;

contacting (1) a first portion of the sample with an agonist of GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with an RTK targeted therapeutic if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or 95% or higher.

In another aspect, provided herein is a method of treating a human subject diagnosed with cancer mediated by RTK signaling, wherein cancer cells of the subject have been tested in a method comprising:

culturing a sample of viable cancer cells obtained from the subject;

contacting (1) a first portion of the sample with an agonist of GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject a targeted therapeutic that inhibits RTK signaling if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or 95% or higher.

In some embodiments, the inhibitor of RTK signaling is a protein, peptide, lipid, nucleic acid, metabolite, ligand, reagent, organic molecule, signaling factor, growth factor, biochemical, or combinations thereof.

Various RTK inhibitors are known in the art. Exemplary (non-limiting) RTKs and their corresponding inhibitors are described in Table 1.

TABLE 1

| RTK family | Family members | Exemplary Inhibitors |
|---|---|---|
| Epidermal growth | EGFR (ERBB1, HER1), HER2 | erlotinib, gefitinib, lapatinib, |
| factor receptor | (ERBB2), HER3 (ERBB3), | vandetanib, afatinib, panitumumab, |
| (EGFR)/ERBB | HER4 (ERBB4) | cetuximab, brigatinib, icotinib, |
| | | osimertinib, neratinib, zalutumumab, |
| | | nimotuzumab, matuzumab, |
| | | pertuzumab, trastuzumab, |
| | | dacomitinib, acomitinib, BIBW2992, |
| | | tesevatinib, amuvatinib, |
| | | necitumumab, REGN955, MM-151, |
| | | nazartinib, ASP8273, olmutinib, |
| | | TDM1, MEDI4276, ZW25, ZW33, |
| | | tucatinib, rociletinib, ibrutinib. DS- |
| | | 8201, TAS07828, XMT-1522, TAK- |
| | | 788, Sym013, LIM716, |
| | | seribantumab, AMG888, |
| | | lumretuzumab, PF-06804103, |
| | | ARX788, poziotinib, pyrotinib, |
| | | duligotuzuman, MCLA-128, MM- |
| | | 111 |
| Muscle-specific | MUSK | |
| kinase (MuSK) | | |
| Hepatocyte growth | MET (c-Met), RON | cabozantinib, tivantinib, crizotinib |
| factor receptor | | (PF-2341066), tepotinib, capmatinib, |
| (HGFR) | | savolitinib, K252a, SU11274, PHA- |
| | | 665752, ARQ197, foretinib, |
| | | SGX523, MP470, AV229, AMG102, |
| | | CGEN241, DN30, OA5D5, |
| | | rilotumumab, onartuzumab, |
| | | SAR125844, embetuzumab, ABBV- |
| | | 399, sym015, ficlatuzumab, |
| | | merestinib, JNJ-61186372, altiratinib, |
| | | Indo5, BMS-754807, BMS-777607, |
| | | glesatinib |
| Nerve growth | TRKA, TRKB, TRKC | CEP-751, ANA-12, cyclotraxin B, |
| factor receptor | | gossypetin, K252a, entrectinib, |
| (NGFR) | | larotrectinib, LOXO-101 |
| Fibroblast growth | FGFR1, FGFR2, FGFR3, | dovotinib, lenvatinib, ponatinib, |
| factor receptor | FGFR4 | regorafenib, lucitanib, cediranib, |
| (FGFR) | | intedanib, brivanib, PD173074, |
| | | AZD4547, BGJ398, JNJ42756493, |
| | | GP369, BAY1187982, MFGR1877S, |
| | | FP1039, pazopanib, erdafitinib, |
| | | Debio-1347, B-701, fisogatinib, |
| | | FIIN-2, FIIN-3, BLU9931, |
| | | LY2874455, LY3076226, sunitinib, |
| | | vandetanib |
| Insulin receptor | IGFR (IGF-IR), IR | AG538, AG1024, NVP-AEW541, |
| (IR) | | figitumumab, linsitinib, dalotuzumab, |
| | | MEDI-573, teprotumumab, |
| | | ganitumab, ceritinib, MM-141 |
| Colon carcinoma | CCK4 (PTK7) | cofetuzumab pelidotin (PF- |
| kinase (CCK) | | 06647020) |
| Ephrin receptor | EPHA1, EPHA2, EPHA3, | dasatinib, nilotinib, NVP-BHG712, |
| (EphR) | EPHA4, EPHA5, EPHA6, | sitravatinib, ALW-II-41-27, |
| | EPHA7, EPHA8, EPHA9, | tesevatinib, JI-101, 123C4 |
| | EPHA10 EPHB1, EPHB2, | |
| | EPHB3, EPHB4, EPHB5, | |
| | EPHB6 | |
| Receptor related to | RYK | |
| tyrosine kinases | | |
| (RYK) | | |
| Rearranged during | RET | sorafenib, vandetanib, lenvalinib, |
| transfection (RET) | | apatinib, AST487, amuvatinib, |
| | | alectinib, dovitinib, pazopanib, |
| | | ponatinib, sunitinib |
| RPTK expressed | ROS | crizotinib, entrectinib, lorlatinib, |
| in some epithelial | | ceritinib, TPX-0005, DS-6051b, |
| cell types (ROS) | | cabozantinib, merestinib |
| Platelet-derived | PDGFRα, PDGFRβ, KIT (c- | imatinib, dasatinib, sunitinib, |
| growth factor | KIT, CD117), FLT3 (CD135), | linifanib, nilotinib, KTN0182A, |
| receptor (PDGFR) | M-CSFR (CD115, CSFR1R) | gilteritinib, quizartinib, midostaurin, |
| | | pazopanib, sorafenib, lestaurtinib, |
| | | ripretinib, masitinib, avapritinib, |
| | | dovitinib, pexidartinib, telatinib, |
| | | motesanib, PLX7486, ARRY386, |
| | | JNJ-40346527, BLZ945, |
| | | emactuzumab, AMG820, IMC-CS4, |
| | | cabiralizumab, JI-101, lenvatinib, |
| | | apatinib, cabozantinib, CHMFL-KIT- |
| | | 033, SU14813, AST487, Ki20227, |
| | | amuvatinib, OSI-930, flumatinib, |
| | | toceranib, AZD3229, AC710, |
| | | AZD2932, ICK03, PLX647, c-Kit- |
| | | IN-3 |
| Discoidin domain | DDR1, DDR2 | nilotinib |
| receptor (DDR) | | |
| Leukocyte | LTK, ALK | ceritinib |
| tyrosine kinase | | |
| (LTK) | | |
| Vascular | VEGFR1, VEGFR2 (KDR), | sunitinib, sorafenib, cabozantinib, |
| endothelial growth | VEGFR3 (FLT4) | cediranib, vatalanib, vandetanib, |
| factor receptor | | dovotinib, tesevatinib, JI-101, |
| (VEGFR) | | lenvatinib, apatinib, pazopanib, |
| | | linifanib, ripretinib, SU14813, |
| | | Ki20227, telatinib, motesanib, |
| | | toceranib, AZD2932, bevacizumab |
| Tyrosine kinase | TIE1, TIE2, TEK | rebastinib, BAY-826 |
| receptor in | | |
| endothelial cells | | |
| (TIE) | | |
| AXL | AXL, MER, TYRO3 | bemcentinib, R428 (BGB324), |
| | | YW327.6S2, GL2I.T, TP-0903, |
| | | LY2801653, amuvatinib, bosutinib, |
| | | MGCD265, ASP2215, cabozantinib, |
| | | foretinib, SGI-7079, gilteritinib, |
| | | merestinib, gilteritinib, BGB324, |
| | | HuMax-AXL-ADC |
| Receptor orphan | ROR1, ROR2 | UC-961 |
| (ROR) | | |
| Lemur (LMR) | AATYK1, AATYK2, AATYK3 | |
| RTK106 | RTK106 | |

Once an RTK inhibitor is determined to affect GPCR signaling in a patient's cancer cells, e.g., as reflected in an output value that is equal to or greater than a pre-determined cut-off or an output value percentage that is equal to or greater than 30%, more preferably equal to or greater than 50%, the cancer patient is selected for treatment with an RTK targeted therapeutic. In some embodiments, the RTK inhibitor and RTK targeted therapeutic are the same compound. Alternatively, once GPCR signaling in the patient's cancer cells have been determined to be responsive to RTK inhibition, the RTK targeted therapeutic used to treat the patient need not be the same compound as the RTK inhibitor. Accordingly, in other embodiments, the RTK inhibitor and RTK targeted therapeutic are different compounds.

In some embodiments, the RTK inhibitor and/or RTK targeted therapeutic is an inhibitor of a member of the EGFR/ERBB, MuSK, HGFR, NGFR, FGFR, IR, CCK, EphR, RYK, RET, ROS, PDGFR, DDR, LTK, VEGFR, TIE, AXL, ROR, LMR, or RTK106 family. In one embodiment, the RTK inhibitor and/or RTK targeted therapeutic is an inhibitor of a member of the ErbB family. In a specific embodiment, the RTK inhibitor and/or RTK targeted therapeutic is an inhibitor of HER2. In a further specific embodiment, the RTK inhibitor and/or RTK targeted therapeutic is neratinib.

GPCRs expressed in vertebrates can be divided into four classes based on sequence similarity and function, including Class A (rhodopsin-like), Class B (secretin receptor family), Class C (metabotropic glutamate receptors), and Class F (frizzled/smoothened receptors). Subclasses of GPCRs which can be targeted in the present methods are described in, e.g., Alexander et al., Br J Pharmacol. 2019; 176 S1: S21-S141 and www.guidetopharmacology.org, and include, but are not limited to, 5-hydroxytryptamine receptors, acetylcholine receptors (muscarinic), adenosine receptors, adhesion class GPCRs, adrenoceptors, angiotensin receptors, apelin receptor, bile acid receptor, bombesin receptors, bradykinin receptors, calcitonin receptors, calcium-sensing receptor, cannabinoid receptors, chemerin receptors, chemokine receptors, cholecystokinin receptors, class frizzled GPCRs, complement peptide receptors, corticotropin-releasing factor receptors, dopamine receptors, endothelin receptors, G protein-coupled estrogen receptor, formylpeptide receptors, free fatty acid receptors, GABA_{B} receptors, galanin receptors, ghrelin receptor, glucagon receptor family, glycoprotein hormone receptors, gonadotrophin-releasing hormone receptors, GPR18, GPR55, GPR119, histamine receptors, hydroxycarboxylic acid receptors, kisspeptin receptor, leukotriene receptors, lysophospholipid receptors, melanin-concentrating hormone receptors, melatonin receptors, metabotropic glutamate receptors, motilin receptor, neuromedin U receptors, neuropeptide FF/neuropeptide AF receptors, neuropeptide S receptor, neuropeptide W/neuropeptide B receptors, neuropeptide Y receptors, neurotensin receptors, opioid receptors, orexin receptors, oxoglutarate receptor, P2Y receptors, parathyroid hormone receptors, platelet-activating factor receptor, prokineticin receptors, prolactin-releasing peptide receptor, prostanoid receptors, proteinase-activated receptors, somatostatin receptors, succinate receptor, tachykinin receptors, thyrotropin-releasing hormone receptors, trace amine receptor, urotensin receptor, vasopressin and oxytocin receptors, and VIP and PACAP receptors. Additional GPCRs include olfactory receptors, orphan receptors (Class A-C orphans), opsin receptor, taste 1 and 2 receptors, and other 7-transmembrane proteins such as GPR107, GPR137, TPRA1, GPR143, and GPR157.

Suitable agonists of the GPCRs and GPCR subclasses described above are well-known in the art and can be used to stimulate the respective GPCRs in the methods described herein.

In some embodiments, the GPCR is a lysophospholipid GPCR. Lysophospholipid GPCRs are activated by lysophospholipids, which can have a glycerol or sphingoid backbone and have a single carbon chain and polar headgroup. This class of receptors is implicated in a broad range of cellular functions, including proliferation, survival, migration, adhesion, and Ca homeostasis.

Accordingly, in one embodiment, provided herein is a method of selecting a human subject diagnosed with cancer for treatment with a RAS node (e.g., PI3K, AKT, mTOR, RAF, MEK, ERK, BCL) targeted therapeutic, the method comprising:

culturing a sample of viable cancer cells obtained from the subject;

contacting (1) a first portion of the sample with an agonist of lysophospholipid GPCR signaling and an inhibitor of a RAS node, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

selecting the subject for treatment with a RAS node targeted therapeutic which inhibits the same RAS node as the inhibitor if the output value that characterizes the change in cell adhesion or attachment is equal to or greater than a pre-determined cut-off value.

In another embodiment, provided herein is a method of treating a human subject diagnosed with cancer mediated by a RAS node (e.g., PI3K, AKT, mTOR, RAF, MEK, ERK, BCL), wherein cancer cells of the subject have been tested in a method comprising:

culturing a sample of viable cancer cells obtained from the subject;

contacting (1) a first portion of the sample with an agonist of lysophospholipid GPCR signaling and an inhibitor of a RAS node, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

administering to the subject a RAS node targeted therapeutic which inhibits the same RAS node as the inhibitor if the output value that characterizes the change in cell adhesion or attachment is equal to or greater than a pre-determined cut-off value.

In another embodiment, provided herein is a method of selecting a human subject diagnosed with cancer for treatment with a RAS node (e.g., PI3K, AKT, mTOR, RAF, MEK, ERK, BCL) targeted therapeutic, the method comprising:

culturing a sample of viable cancer cells obtained from the subject;

contacting (1) a first portion of the sample with an agonist of lysophospholipid GPCR signaling and an inhibitor of a RAS node and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with a RAS node targeted therapeutic which inhibits the same RAS node as the inhibitor if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or 95% or higher.

In another embodiment, provided herein is a method of treating a human subject diagnosed with cancer mediated by a RAS node (e.g., PI3K, AKT, mTOR, RAF, MEK, ERK, BCL), wherein cancer cells of the subject have been tested in a method comprising:

culturing a sample of viable cancer cells obtained from the subject;

contacting (1) a first portion of the sample with an agonist of lysophospholipid GPCR signaling and an inhibitor of a RAS node, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject a RAS node targeted therapeutic which inhibits the same RAS node as the inhibitor if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or 95% or higher.

In another embodiment, provided herein is a method of selecting a human subject diagnosed with cancer for treatment with a RTK targeted therapeutic (e.g., an ErbB family member targeted therapeutic such as a HER2 targeted therapeutic), the method comprising:

culturing a sample of viable cancer cells obtained from the subject;

contacting (1) a first portion of the sample with an agonist of lysophospholipid GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

selecting the subject for treatment with an RTK targeted therapeutic which inhibits the same RTK as the inhibitor if the output value that characterizes the change in cell adhesion or attachment is equal to or greater than a pre-determined cut-off value.

In another embodiment, provided herein is a method of treating a human subject diagnosed with cancer mediated by RTK signaling, wherein cancer cells of the subject have been tested in a method comprising:

culturing a sample of viable cancer cells obtained from the subject;

contacting (1) a first portion of the sample with an agonist of lysophospholipid GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value that characterizes whether a change in cell adhesion or attachment has occurred in the first portion compared to the second portion; and

administering to the subject an RTK targeted therapeutic which inhibits the same RTK as the inhibitor if the output value that characterizes the change in cell adhesion or attachment is equal to or greater than a pre-determined cut-off value.

In another embodiment, provided herein is a method of selecting a human subject diagnosed with cancer for treatment with a RTK targeted therapeutic (e.g., an ErbB family member targeted therapeutic such as a HER2 targeted therapeutic), the method comprising:

culturing a sample of viable cancer cells obtained from the subject;

contacting (1) a first portion of the sample with an agonist of lysophospholipid GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

selecting the subject for treatment with an RTK targeted therapeutic which inhibits the same RTK as the inhibitor if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or 95% or higher.

In another embodiment, provided herein is a method of treating a human subject diagnosed with cancer mediated by RTK signaling, wherein cancer cells of the subject have been tested in a method comprising:

culturing a sample of viable cancer cells obtained from the subject;

contacting (1) a first portion of the sample with an agonist of lysophospholipid GPCR signaling and an inhibitor of RTK signaling, and (2) a second portion of the sample with the agonist alone;

continuously measuring cell adhesion or attachment of the viable cancer cells in the first and second portions;

determining by mathematical analysis of the continuous measurements an output value percentage that characterizes whether a change in cell adhesion or attachment has occurred in the first portion compared to the second portion; and

administering to the subject an RTK targeted therapeutic which inhibits the same RTK as the inhibitor if the output value percentage is equal to or greater than 30%, for example equal to or greater than 35%, or for example equal to or greater than 40%, or for example equal to or greater than 45%, or for example equal to or greater than 50%, or for example equal to or greater than 55%, or for example equal to or greater than 60%, or for example equal to or greater than 65%, or for example equal to or greater than 70%, or for example equal to or greater than 75%, or for example equal to or greater than 80%, or for example equal to or greater than 85%, or for example equal to or greater than 90%, or 95% or higher.

In some embodiments of the methods described herein, the inhibitor of the RAS node and the RAS node targeted therapeutic are the same compound. In other embodiments, the inhibitor of the RAS node and the RAS node targeted therapeutic are different compounds. In some embodiments, the inhibitor of the RTK signaling and RTK targeted therapeutic are the same compound. In other embodiments, the inhibitor of RTK signaling and RTK targeted therapeutic are different compounds.

In some embodiments of the methods described herein, the lysophospholipid GPCR is selected from the group consisting of a lysophosphatidic acid (LPA) receptor, a sphingosine 1-phosphate (S1P) receptor, a lysophosphatidyl inositol (LPI) receptor, and a lysophosphatidylserine (LyPS) receptor. In some embodiments, the lysophospholipid GPCR is an endothelial differentiation gene (EDG) GPCR, for example, an LPA receptor (LPAR1-3) or an S1P receptor (S1PR1-5). In some embodiments, the lysophospholipid GPCR is a P2Y family GPCR (LPAR4-6).

In some embodiments, the LPA receptor (LPAR) is selected from the group consisting of LPAR1, LPAR2, LPAR3, LPAR4, LPAR5, and LPAR6 (also referred to in the art as LPA₁, LPA₂, LPA₃, LPA₄, LPAs, LPA₆). In some embodiments, the S1P receptor (S1PR) is selected from the group consisting of S1PR1, S1PR2, S1PR3, S1PR4, and S1PR5 (also referred to in the art as S1P₁, S1P₂, S1P₃, S1P₄, and S1P₅). In some embodiments, the LPI receptor is GPR55. In some embodiments, the LyPS receptor is selected from the group consisting of LyPS1 (GPR34), LyPS2 (P2Y10), LyPS2L, and LyPS3 (GPR174).

In some embodiments, the agonist of GPCR signaling is a protein, peptide, lipid, nucleic acid, metabolite, ligand, reagent, organic molecule, signaling factor, growth factor, biochemical, or combinations thereof.

In embodiments for which the GPCR is an LPA receptor, exemplary agonists include, but are not limited to, LPA, FAP-10, FAP-12, and OMPT. Non-limiting examples of LPA which are suitable for use as agonists include, e.g., LPA 18:0, LPA 18:1, LPA 18:2, LPA 20:4, and LPA 16:0.

In embodiments for which the GPCR is an S1P receptor, exemplary agonists include, but are not limited to, S1P, FTY720, BAF312, A971432, ceralifimod, CS2100, CYM50260, CYM50308, CYM5442, CYM5520, CYM5541, GSK2018682, RP001 hydrochloride, SEW2871, TC-G1006, and TC-SP14.

In embodiments for which the GPCR is LPI receptor (GPR55), exemplary agonists include, but are not limited to, LPI and 2-arachidonoyl LPI.

In embodiments for which the GPCR is a LyPS receptor, exemplary agonists include, but are not limited to, LyPS and analogues thereof, e.g., as described in Ikubo et al. J Med Chem 2015; 58:4204-19.

In some embodiments of the methods described herein, the viable cancer cells are contacted with a third portion of the sample containing the therapeutic agent alone. In some embodiments, the step of determining by mathematical analysis of the continuous measurements an output value that characterizes whether a change in cell adhesion or attachment has occurred involves a comparison of the first portion with the second portion and third portion of the sample.

In one embodiment, the GPCR signaling pathway is abnormally active in the subject's cancer cells. Methods for determining abnormally active signaling are described in the subsections below.

Predetermined cut-off values for use in the methods provided herein are described in the subsections below.

In one embodiment, cell adhesion or attachment is measured using an impedance biosensor or an optical biosensor. Use of biosensors is described in further detail in the subsections below.

In one embodiment, the cancer is selected from the group consisting of breast cancer, lung cancer, colorectal cancer, bladder cancer, kidney cancer, ovarian cancer and leukemia. Additional suitable cancers are described in the subsections below.

In some embodiments of the methods described herein, the subject's cancer cells express a non-mutated (i.e., wild-type) RAS node protein. In one embodiment, the subject's cancer cells express non-mutated (i.e., wild-type) PI3K enzyme. In another embodiment, the subject's cancer cells express mutated PI3K enzyme. In one embodiment, the subject's cancer cells express non-mutated (i.e., wild-type) PI3K, ERK, MEK, RAF, BCL, and/or mTOR. In one embodiment, the subject's cancer cells express non-elevated levels of RTK (i.e., RTK negative cancer cells). In one embodiment, the subject's cancer cells express non-elevated levels of HER2 (i.e., HER2 negative cancer cells). In one embodiment, the subject's cancer cells are HER2 negative and express non-mutated (i.e., wild-type) PI3K, ERK, MEK, RAF, BCL, and/or mTOR. In some embodiments, the subject's cancer cells express a non-mutated (i.e., wild-type) GPCR. Accordingly, in one embodiment, the GPCR activated by the agonist of GPCR signaling in the methods described herein is not mutated (i.e., wild-type). In another embodiment, the subject's cancer cells express non-elevated levels of the GPCR. That is, the GPCR activated by the agonist of GPCR signaling in the methods described herein need not be overexpressed, or expressed above a statistically significant level above normal.

In one embodiment, the sample of viable cancer cells is cultured in a media comprising growth factors and free of serum. In one embodiment, the sample of viable cancer cells is also cultured in a media comprising an anti-apoptotic agent and free of serum. Culture conditions and culturing of cells is described in further detail in the subsections below.

### Methods of Measuring GCPR Signaling Pathway Ultra-Sensitivity

As discussed in relation to the methods described herein, in some embodiments, the GPCR signaling pathway in the patient's cancer cells is abnormally active (i.e., ultra-sensitive). Accordingly, provided herein are methods for determining whether a GPCR signaling pathway in a patient's cells is abnormally ultra-sensitive. An ultra-sensitive pathway is one in which the change of only a very low level of cellular input, such as a signaling pathway agonist (e.g., a receptor ligand concentration change from 1 nM to 10 nM) is capable of causing a change from a low level (e.g., 10% of cellular output) of pathway activation to a very high level of pathway activation responsiveness (e.g., 90% cellular output). A signaling pathway in a patient's cancer cells that is abnormally ultra-sensitive is likely to be involved in driving the disease process (e.g., tumor growth), even if there are additional aberrant signaling pathways in the cancer cells. Thus, identifying a signaling pathway in a patient's cancer calls that is abnormally ultra-sensitive is an effective means for selecting therapeutically effective treatment regimens. An embodiment of how ultra-sensitivity of cancer cells to a signaling pathway is described in Example 2.

Accordingly, in one aspect, provided herein is a method of determining whether a human subject diagnosed with cancer has abnormally active GPCR signaling comprising,

culturing a sample comprising viable cancer cells obtained from the subject;

contacting the sample with an agonist of a GPCR signaling pathway;

continuously measuring cell adhesion or attachment of the viable cancer cells in a portion of the sample contacted with the agonist relative to a portion of the sample that has not been contacted with the agonist; and

determining by mathematical analysis of the continuous measurements sensitivity of the sample to the agonist and an output value for the agonist that characterizes whether a change in cell adhesion or attachment has occurred in the portion of the sample contacted with the agonist, as compared to the portion of the sample not contacted with the agonist, wherein the GPCR signaling pathway is considered abnormally active when the output value for the agonist is equal to or greater than a pre-determined cut-off value.

In another aspect, provided herein is a method of determining whether a human subject diagnosed with cancer has abnormally active GPCR signaling comprising,

culturing a sample comprising viable cancer cells obtained from the subject;

contacting the sample with an agonist of a GPCR signaling pathway, wherein a portion of the sample is contacted with a higher concentration of the agonist and a portion of the sample is contacted with a lower concentration of the agonist;

continuously measuring cell adhesion or attachment of the viable cancer cells in the portion of the sample contacted with a higher concentration of the agonist, relative to the portion of the sample contacted with the lower concentration of the agonist; and

determining by mathematical analysis of the continuous measurements the sensitivity of the signaling pathway to the agonist, wherein the GPCR signaling pathway is considered abnormally active when the signaling pathway is ultra-sensitive to the agonist.

In one embodiment, the higher concentration of the activator is EC90 (i.e., an Effective Concentration 90) and the lower concentration of the activator is EC10 (i.e., an Effective Concentration 10). As used herein, an EC90 is that concentration of activator that gives 90% of the maximal response for the activator on the subject's cells. As used herein, the EC10 is that concentration of activator that gives 10% of the maximal response for the activator on the subject's cells. In one embodiment, an EC90:EC10 ratio of less than 81 indicates that the signaling pathway is ultra-sensitive to the activator.

In other embodiments, the higher concentration of the activator is 2-fold, 3-fold, 4-fold, 5-fold, 10-fold, 20-fold, 30-fold, 40-fold, 50-fold, 60-fold, 70-fold or more up to 80-fold more than the lower concentration of the activator.

In another embodiment, the higher concentration of the activator is the only concentration that is used and is determined from comparing results of a small population of patients who are ultra-sensitive with a small population of patients who are not ultra-sensitive by any of the embodiments described herein. Accordingly, in another embodiment, the methods of the disclosure involving measuring signaling pathway ultrasensitivity comprise a contacting step (between the culturing step and the continuously measuring cell adhesion or attachment step) of contacting the sample with an agonist of a GPCR signaling pathway, so as to activate the signaling pathway as measured by an effect on cell adhesion or attachment, wherein the concentration of the activator used is a concentration that has been determined to identify ultrasensitivity of the signaling pathway.

In one embodiment, the sensitivity of the signaling pathway to the activator is determined using the Hill equation to determine a Hill Coefficient.

The Hill equation is known in the art and can be expressed most simply as:

$$Output = \frac{{Input}^{n}}{{K}^{n} + {Input}^{n}}$$

$$And n = \frac{Log [ 81 ]}{Log [ EC 90 / EC 10 ]}$$

wherein:
Input is the input concentration. It can be expressed in many different forms such as pM, nM, M, mM, M, ng/mL, %, etc.
K_{0.5} is the half-maximal concentration constant. It may also be referred to as K_{half}. It is the substrate concentration that gives rise to a reaction that is 50% complete.
n is the Hill coefficient.

In one embodiment, a Hill Coefficient value of greater than one indicates that the signaling pathway is ultra-sensitive to the activator.

The Hill equation expressed in an alternative manner:

$$θ = \frac{{[ L ]}^{n}}{{K}_{d} + {[ L ]}^{n}}$$

Wherein

θ—Fraction of the activity derived from the pathway
[L]—Free (unbound) ligand (activator) concentration
K_{d}—Apparent dissociation constant derived from the law of mass action (the equilibrium constant for dissociation), which is equal to the ratio of the dissociation rate of the ligand-receptor complex to its association rate

$$( {K}_{d} = \frac{{k}_{d}}{{k}_{a}} ) .$$

n—the Hill coefficient

One of ordinary skill in the art would recognize that the equation above is useful in creating a linear plot of

$$log ( \frac{θ}{1 - θ} )$$

versus log L that yields a linear plot wherein the slope is n, the Hill coefficient.

One of ordinary skill in the art would also recognize that EC₉₀/EC₁₀<81 represents an ultrasensitive signaling instance, wherein the smaller the EC₉₀/EC₁₀ ratio, the greater the ultrasensitivity.

In one embodiment, the GPCR signaling pathway is a lysophospholipid GPCR signaling pathway, for example, a lysophospholipid GCPR selected from the group consisting of LPAR, S1PR, LPIR (GPR55), and a LyPSR. In some embodiments, the LPA receptor (LPAR) is selected from the group consisting of LPAR1, LPAR2, LPAR3, LPAR4, LPAR5, and LPAR6. In some embodiments, the S1P receptor (S1PR) is selected from the group consisting of S1PR1, S1PR2, S1PR3, S1PR4, and S1PR5. In some embodiments, the LyPS receptor is selected from the group consisting of LyPS1 (GPR34), LyPS2 (P2Y10), LyPS2L, and LyPS3 (GPR174).

In some embodiments, the agonist of GPCR signaling (e.g., lysophospholipid GPCR signaling) is a protein, peptide, lipid, nucleic acid, metabolite, ligand, reagent, organic molecule, signaling factor, growth factor, biochemical, or combinations thereof. Any GPCR agonist known in the art is suitable for determining whether the corresponding GPCR signaling pathway is ultrasensitive in a subject's cells. Exemplary lysophospholipid GPCR agonists suitable for use are described in the preceding subsection.

In some embodiments of the methods described herein, the responsiveness of the abnormally active GPCR signaling pathway to a RAS node or RTK targeted therapeutic is tested after first determining whether the GPCR signaling pathway (e.g., lysophospholipid GPCR signaling pathway) is abnormally active. In other embodiments, the sensitivity of the patient's cells to an agonist of GPCR signaling is tested simultaneously with a determination of whether an inhibitor of a RAS node or RTK signaling affects the GPCR signaling pathway.

In one embodiment, cell adhesion or attachment is measured using an impedance biosensor or an optical biosensor. Use of biosensors is described in further detail in the subsections below.

In one embodiment, the cancer is selected from the group consisting of breast cancer, lung cancer, colorectal cancer, bladder cancer, kidney cancer, ovarian cancer and leukemia. Additional suitable cancers are described in the subsections below.

In one embodiment, the subject's cancer cells express a non-mutated (i.e., wild-type) RAS node protein. In one embodiment, the subject's cancer cells express non-mutated (i.e., wild-type) PI3K enzyme. In another embodiment, the subject's cancer cells express mutated PI3K enzyme. In one embodiment, the subject's cancer cells express non-mutated (i.e., wild-type) PI3K, ERK, MEK, RAF, BCL, and/or mTOR. In one embodiment, the subject's cancer cells express non-elevated levels of RTK (i.e., RTK negative cancer cells). In one embodiment, the subject's cancer cells express non-elevated levels of HER2 (i.e., HER2 negative cancer cells). In one embodiment, the subject's cancer cells are HER2 negative and express non-mutated (i.e., wild-type) PI3K, ERK, MEK, RAF, BCL, and/or mTOR. In one embodiment, the GPCR activated by the agonist of GPCR signaling in the methods described herein is not mutated (i.e., wild-type). In another embodiment, the subject's cancer cells express non-elevated levels of the GPCR activated by the agonist of GPCR signaling.

In one embodiment, the sample of viable cancer cells is cultured in a media comprising growth factors and free of serum. In another embodiment, the sample of viable cancer cells is also cultured in a media comprising an anti-apoptotic agent and free of serum. Culture conditions and culturing of cells is described in further detail in the subsections below.

In some embodiments, the response of a sample to one or more of these agents can also be measured in the presence or absence of a growth factor that perturbs cell proliferation or of an anti-apoptotic agent. Growth factors that perturb cell proliferation include growth hormone, epidermal growth factor, vascular endothelial growth factor, platelet derived growth factor, hepatocyte growth factor, transforming growth factor, fibroblast growth factor, nerve growth factors, and others known to those practiced in the art. Anti-apoptotic agents include compounds that regulate anti-apoptotic proteins or pathways (e.g., taxols on Bcl-2 protein activity and Gefitinib for control of the anti-apoptotic Ras signaling cascade).

### Sample Preparation and Culturing

Embodiments of the invention include systems, kits, and methods to determine the effectiveness of a targeted therapeutic agent (e.g., a RAS node or RTK targeted therapeutic agent), monitor the effectiveness of the targeted therapeutic agent, or identify a dose of a targeted therapeutic agent when administered to a subject's diseased cells.

Traditionally, disease has been classified by the tissue or organ that the disease affects. Due to better knowledge of the underlying mechanisms (e.g., hyperactive RTK or GPCR signaling, genetic, autoimmune response, etc.), it is now understood that diseases which affect the same tissue/organ, or produce the same symptoms, may have different etiologies and may have heterogeneous gene expression profiles. In addition, it has been shown in many diseases that there are responders and non-responders to therapeutic agents. In embodiments, any disease type, for which responders and non-responders are identified, can be employed in the methods herein in order to predict or prognosticate whether a particular therapeutic drug combination of drugs will be effective for a particular individual, e.g. a determination whether the individual is a responder or a non-responder.

One example of a disease type that is known to be heterogeneous in nature and to have responders and many non-responders is cancer. Cancer is typically classified according to tissue type. However, a more accurate description of the heterogeneity of cancer is reflected in the different mutations of the different cancers. An even more accurate description of the heterogeneity of cancer is the actual functional, physiological result of the mutation in a particular patient's cells. For instance, breast cancer has different types and different mutations that cause cancer of this organ. Outcomes and treatments can be different based on whether the mutation causing the cancer is a gain of function (e.g., proto-oncogene causing increase protein production) or loss of function mutation (e.g., tumor suppressor) and in which gene. Due to the heterogeneity of a particular cancer, it would be expected that there would a heterogeneous response to a particular therapeutic agent. Embodiments of this invention allow the testing of a particular subject's cancer cells to a therapeutic agent or a panel of therapeutic agents to determine the efficacy of a specific therapeutic agent or the most effective therapeutic agent for a particular subject's cancer to select a treatment for the subject.

Embodiments of the invention include disease cell samples of cancer cells from individual subjects. Such cancer cells can be derived from, but not limited to, Acute Lymphoblastic Leukemia (ALL), Acute Myeloid Leukemia (AML), Adrenocortical Carcinoma, Anal Cancer, Appendix Cancer, Astrocytomas, basal cell carcinoma, Extrahepatic Bile Duct Cancer, Bladder Cancer, Bone Cancer, Osteosarcoma, Malignant Fibrous Histiocytoma, Brain Stem Glioma, Central Nervous System Atypical Teratoid/Rhabdoid Tumor, Central Nervous System Embryonal Tumors, Central Nervous System Germ Cell Tumors, Craniopharyngioma, Ependymoblastoma, Ependymoma, Medulloblastoma, Medulloepithelioma, breast cancer, Pineal Parenchymal Tumors of Intermediate Differentiation, Supratentorial Primitive Neuroectodermal Tumors, Pineoblastoma, Bronchial Tumors, Carcinoid Tumor, Cervical Cancer, Chronic Lymphocytic Leukemia (CLL), Chronic Myelogenous Leukemia (CML), Chronic Myeloproliferative Disorders, Colon Cancer, Colorectal Cancer, Cutaneous T-Cell Lymphoma, Ductal Carcinoma In Situ (DCIS), Endometrial Cancer, Esophageal Cancer, Esthesioneuroblastoma, Ewing Sarcoma, Extragonadal Germ Cell Tumor, Intraocular Melanoma, Retinoblastoma, fibrous histocytoma, Gallbladder Cancer, Gastric Cancer, Gastrointestinal Carcinoid Tumor, Gastrointestinal Stromal Tumors (GIST), Gestational Trophoblastic Tumor, Glioma, Hairy Cell Leukemia, Heart Cancer, Hepatocellular Cancer, Langerhans Cell Histiocytosis, Hodgkin Lymphoma, Hypopharyngeal Cancer, islet cell tumors, Kaposi sarcoma, renal cell cancer, Laryngeal Cancer, Lip Cancer, Liver Cancer, Lobular Carcinoma In Situ (LCIS), Lung Cancer, Merkel cell carcinoma, Melanoma, mesothelioma, mouth cancer, multiple myeloma, Nasal Cavity and Paranasal Sinus Cancer, Nasopharyngeal Cancer, Neuroblastoma, Non-Hodgkin Lymphoma, Non-Small Cell Lung Cancer, Oral Cavity Cancer, Oropharyngeal Cancer, Ovarian Cancer, Pancreatic Cancer, Papillomatosis, Paraganglioma, Parathyroid Cancer, Penile Cancer, Pharyngeal Cancer, Pheochromocytoma, Pineal Parenchymal, Pituitary Tumor, Pleuropulmonary Blastoma, Prostate Cancer, rectal cancer, rhabdomyosarcoma, salivary gland cancer, squamous cell carcinoma, small intestinal cancer, testicular cancer, throat cancer, thyroid cancer, ureter cancer, urethral cancer, uterine cancer, vaginal cancer, vulvar cancer, and Wilm's tumor.

Autoimmune diseases are characterized by increased inflammation due to immune system activation against self antigens. Current therapies target immune system cells such as B cells and inflammatory molecules such as anti TNFα. Therapies can be broadly characterized as immune modulating or immunosuppressant. Drugs may be targeted to particular molecules such as TNF alpha, Integrins, sphingosine receptors, and interleukins. Other drugs act as anti-inflammatory agents such as corticosteroids. In yet other cases, drugs are immunosuppressants such as mercaptopurines and cyclophosphamide. With respect to autoimmune conditions, peripheral blood cells may be examined for the response to a certain therapeutic. In other embodiments, tissue samples of the site of inflammation, for example, synovial tissue in rheumatoid arthritis or colon tissue for ulcerative colitis.

For example, some patients with rheumatoid arthritis are known to be non-responders to anti-TNFα antibodies. In an embodiment, peripheral blood cells can be obtained from a patient suspected as having RA and a decrease in cell signaling ability of the patient's TNF Receptor and associated MAPK pathway can be used to determine whether the patient is likely to be a responder or non-responder to an immunomodulating or immunosuppressant compound. Likewise other therapeutics such as those targeting to IL-6, Interferon alpha, Interferon gamma, and the like may be tested in the same way. In other embodiments, it is known that patients that have multiple sclerosis are nonresponders to interferon beta. Cell samples from subjects can be tested against a panel of drugs to see which if any of the drugs are effective for a particular subject by inducing a change in a cellular physiological parameter. Examples of advantageous outcomes would be a reduction in cellular inflammation parameters, as determined by the American College of Rheumatology (ACR) criteria or an increase in cell adhesion for strengthening the blood-brain barrier function.

In other embodiments, patients may have a disease caused by infection of cells by a microorganism, a foreign body, or a foreign agent. Blood cells or tissue samples infected with a microorganism may be evaluated for responsiveness to various antibiotics, antivirals, or other therapeutic candidates. For example, there are a number of different therapeutic agents for hepatitis C infection that reduce viral function, infected tissue samples can be contacted with one or more therapeutic agents and a change in a cellular physiological parameter is detected. Therapeutic agents are selected that provide a change in a cellular physiological parameter of the infected tissue, and/or a therapeutic agent that provides a change in a cellular physiological parameter at the lowest dose. Outcomes such as increase in cell survival or increase in cell growth would be considered advantageous. In other embodiments where the therapeutic is designed to effect the human cell directly such as by blocking viral entry via a specific receptor type or activation of a cellular pathway, the patient cell could be tested for receptor binding or pathway activation by said therapeutic as described in other embodiments herein.

In embodiments, the cell samples can be obtained before therapy is initiated, during therapy, after therapy, during remission, and upon relapse. The methods as described herein are useful to predict therapeutic efficacy prior to treatment, during treatment, when a patient develops resistance, and upon relapse. The methods of the disclosure are also useful as to predict responders or non-responders to a therapeutic agent or combination of agents.

In certain embodiments, the cells are not contacted or treated with any kind of fixative, or embedded in paraffin or other material, or any detectable label. In other embodiments, it is preferred that the cells remain whole, viable and/or label free. Thus, viable primary cells can be used as the cell sample. In some other embodiments, a cell sample is provided for both the diseased tissue and healthy tissue. In some embodiments, the cell sample is provided in both viable and fixed form. A cell sample provided in fixed form can serve as a control for comparison to the viable cells that are analyzed in accord with the methods as described herein particularly for improved identification and correlation of additional biomarkers.

In other embodiments of the invention, cells from an individual subject are used to determine therapeutic effectiveness. Cells can be collected and isolated by well-known methods (i.e., swab, biopsy, etc.). Both diseased and non-diseased cells can be used. Non-diseased cells can be used as a negative control, a baseline measure, a comparison for measures over time, etc. In embodiments, a control sample of tissue cells from the same subject may also be obtained. A control sample may be taken from another healthy tissue in the subject or from healthy tissue from the same organ as the diseased tissue sample or more preferably healthy tissue is taken from an individual without disease. Diseased cells are cells extracted from a tissue with active disease. In an embodiment, diseased cells can be tumor cells, such as breast cancer cells. Cancerous cells do not necessarily have to be extracted from a tumor. For instance, leukemic cells can be collected from the blood of a patient with leukemia. Cells can be collected from different tissue sites such as the sites of metastasis, circulating tumor cells, primary tumor sites, and recurrent tumor sites, and cellular responsiveness compared to one another. In another embodiment, diseased cells can be extracted from a site of autoimmune disease, such as rheumatoid arthritis. In certain embodiments, the number of cells in each tissue sample is preferably at least about 5000 cells. In other embodiments, the cell number in the tissue sample may range from about 5000 to 1 million cells or greater. Cell samples include isolation from, but are not limited to, blood, blood serum, blood plasma, urine, semen, seminal fluid, seminal plasma, prostatic fluid, pre-ejaculatory fluid (Cowper's fluid), excreta, tears, saliva, sweat, biopsy, ascites, cerebrospinal fluid, lymph, marrow, or hair. In some other embodiments, the cell samples can contain or be derived from patient serum, fractions thereof, organoids, fibroblasts, stromal cells, mesenchymal cells, epithelial cells, white blood cells, red blood cells, B cells, T cells, immune cells, stem cells, or combinations thereof.

In one embodiment, the extraction of cells from a subject is performed at the same location as method of evaluating signaling pathway activity (e.g, the CReMS system described herein) (e.g., at a laboratory, hospital). As such, the cells can be suspended or preserved in a well-known transfer medium to bridge the time from subject to biosensor. In another embodiment, the extraction of cells from a subject is at a different location from the method of evaluating signaling pathway activity (e.g, the CReMS system described herein). Once obtained the cell samples are maintained in a medium that retains the cell viability. Depending on the length of time for transportation to the site of analysis, different media may employed. In embodiments, when transportation of the tissue sample may require up to 10 hours, the media has an osmolality of less than 400 mosm/L and comprises Na+, K+, Mg+, Cl-, Ca+2, glucose, glutamine, histidine, mannitol, and tryptophan, penicillin, streptomycin, contains essential amino acids and may additionally contain non-essential amino acids, vitamins, other organic compounds, trace minerals and inorganic salts, serum, cell extracts, or growth factors, insulin, transferrin, sodium selenite, hydrocortisone, ethanolamine, phosphophorylethanoloamine, triiodothyronine, sodium pyruvate, L-glutamine, to support the proliferation and plating efficiency of human primary cells. Examples of such a media include Celsior media, Roswell Park Memorial Institute medium (RPMI), Hanks Buffered Saline, and McCoy's 5A, Eagle's Essential Minimal Media (EMEM), Dulbecco's modified Eagle's medium (DMEM), Leibovitz L-15, or modifications thereof for the practice of primary cell care. In embodiments, the media and containers are endotoxin free, nonpyrogenic and DNase- and RNase-free.

In other embodiments, the diseased cells obtained from a tissue specimen of an individual subject are extracted using steps that include mincing and enzyme digestion of a tissue specimen, separation of extracted cells by cell type, and/or culturing of the extracted cells. The culturing reagent can include various supplements, for example, patient serum or patient derived factors.

A further aspect includes a method of extracting organoids from a tissue specimen, which can subsequently be used to determine the efficacy of a therapeutic agent in an individual subject. Such a method comprises mincing and enzyme digestion steps. A further aspect includes a method of culturing organoids from a tissue specimen, which can subsequently be used to determine the efficacy of a therapeutic agent in an individual subject. Such a method comprises mincing, enzyme digestion, separation by cell type, and culturing steps. A further aspect may include the specific recombination of the so separated cells to perform the methods described herein.

In certain embodiments, prior to assessing signaling pathway activity in a sample of viable cells from a subject, the cells are first cultured in a media free of serum and any agents that could perturb the GPCR signaling pathway to be assessed such that the cells are synchronized with respect to physiological state and pathway activation. In certain embodiments, the cell sample is cultured in a media free of serum and growth factors. In other embodiments, the cells are cultured in a media that maintains functional cellular cyclic adenosine monophosphate (cAMP), functional thyroid receptors and/or functional G-protein-coupled receptors (or any combination of two or three of the aforementioned properties) in order to support proliferation and plating efficacy of human primary cells.

In one embodiment, the sample of viable cancer cells is cultured in a media comprising growth factors and free of serum. In another embodiment, the sample of viable cancer cells is also cultured in a media comprising an anti-apoptotic agent and free of serum. Non-limiting examples of anti-apoptotic agents include kinase inhibitors, protease inhibitors, stress inhibitors, death receptor inhibitors, cytochrome C inhibitors, anoikis inhibitors, including Rho-associated kinase inhibitors, ALK5 inhibitors, caspase inhibitors, matrix metalloprotease inhibitors, redox buffering agents, reactive oxygen species inhibitors, TNFα inhibitors, TGFβ inhibitors, cytochrome C release inhibitors, carbonic anhydrase antagonists without calcium channel activation, integrin stabilizers, integrin ligands, Fas inhibitors, FasL inhibitors, Bax inhibitors and Apaf-1 inhibitors.

Additional suitable culturing conditions for preparation of a primary cell sample, e.g., a viable cancer cell sample obtained from a subject, are described in detail in PCT Application No. PCT/US2016/057923, published as WO 2017/070353, the entire contents of which is expressly incorporated herein by reference.

### Sample Analysis

Systems and methods of the invention utilize a system referred to herein as a cellular response measurement system (CReMS). CReMS refers to a device (e.g., biosensor) that can quantitatively determine a change in a physiological parameter in a cell, in and between cells, and between cells and the instrumentation device. A change in a physiological parameter is measured by determining change in an analyte (including non-limiting examples such as extracellular matrix, cell signaling molecule, or cell proliferation, tissue, cells, metabolites, catabolites, biomolecules, ions, oxygen, carbon dioxide, carbohydrates, proteins etc.). In some embodiments, the biosensor is measuring a change in the physiological parameter in isolated whole label free viable cells. In some embodiments, a biosensor is selected that can measure an expected change due to the type of therapeutic and/or activator agent.

An example of a CReMS is a biosensor. Examples of biosensors are electrochemical biosensors, electrical biosensors, optical biosensors, mass sensitive biosensors, thermal biosensors, and ISFET biosensors. Electrochemical biosensors measure potentiometric, amperometric and/or voltammetric properties. Electrical biosensors measure surface conductivity, impedance, resistance or electrolyte conductivity. Optical biosensors measure fluorescence, absorption, transmittance, density, refractive index, and reflection. Mass sensitive biosensors measure resonance frequency of piezocrystals. Thermal biosensors measure heat of reaction and adsorption. ISFET biosensors measure ions, elements, and simple molecules like oxygen, carbon dioxide, glucose, and other metabolites of interest in the life sciences. In some embodiments, the biosensor is selected from the group consisting of an impedance device, a photonic crystal device, an optical waveguide device, a surface plasmon resonance device, quartz crystal resonators/microbalances, and a microcantilever device. In some embodiments, an optical biosensor can comprise an optical transducer for converting a molecular recognition or molecular activation event in a living cell, a pathogen, or combinations thereof. In a specific embodiment, the device is an impedance device.

In an example of a biosensor used to measure protein or other in vitro biomolecular interactions, the capture of a specific protein mass is translated into meaningful biochemical and biophysical values. Applying a simple calculation with the captured mass involving the molecular weight of the specific protein captured, the number of moles are evaluated, leading to equilibrium binding constants and other interaction descriptive values known to those experienced in the art. In an example of a biosensor used for cell assays, specific adhesion molecules on the cell surface modulate their attachment and morphology close to the surface of the sensor and other nearby cells upon application of external chemical or other stimulus via specific cellular pathways.

The biosensor can detect these modulations that can be selected in such a way as to be unique to the stimulus and pathway within the cell employed to respond to stimuli. When designed properly, the biosensor result for said cell assay can be exquisitely quantitative in molecular and functional terms. Said biosensor result can be a temporal pattern of response for further uniqueness. Biomolecular activators or perturbants known to turn on and turn off specific pathways within the cell can be used as controls for determining the specificity of the CReMS biosensor signal. Methods for curve deconvolution of the temporal response of the biosensor result (e.g. non-linear Euclidean comparison with control responses) can be applied to further more finely detail specific cellular responses. Use of titrating external stimuli in a cellular biosensor assay can also provide further biochemical and biophysical parameter description.

One example of a label-free sensor is a high frequency quartz resonator or quartz crystal microbalance (QCM) or resonating cantilever. The resonator includes a quartz crystal with a patterned metal electrode upon its surface. The quartz material has well-characterized resonance properties when a voltage is applied. By applying an alternating voltage to the electrodes at a particular frequency, the crystal will oscillate at a characteristic frequency. The oscillation frequency is modulated in quantitative ways when mass is captured on the sensor surface; additional mass results in lower resonator frequency. Therefore, by measuring small changes in the resonant frequency of the quartz oscillator, very small changes in deposited mass can be measured without attaching a label to the biomolecule or cell under study.

Ion Selective Field Effect Transistor (ISFET) devices are miniaturized, nanoscale, devices that are capable of measuring selected ions, elements, and simple molecules like oxygen, carbon dioxide, glucose, and other metabolites of interest in the life sciences. They have been extensively described at the electromechanical operational level as well as at the bioapplication level. To date they have not been described for the use with a specific patient's cells to discern response or resistance or temporal patterns thereof to proposed therapeutic intervention in disease processes.

Optical biosensors are designed to produce a measurable change in some characteristic of light that is coupled to the sensor surface. The advantage of this approach is that a direct physical connection between the excitation source (the source of illumination of the sensor), the detection transducer (a device that gathers reflected or transmitted light), and the transducer surface itself is not required. In other words, there is no need for electrical connections to an optical biosensor, simplifying methods for interfacing the sensor with fluid required for stabilizing and studying most biological systems. Rather than detecting mass directly, all optical biosensors rely on the dielectric permittivity of detected substances to produce a measurable signal. The changes in dielectric permittivity are related to the difference in ratio of the speed of light in free space to that in the medium. This change essentially represents the refractive index of the medium. The refractive index is formally defined as the square root of the dielectric constant of a medium (see Maxwell's equation for more explicit treatment of this relationship). An optical biosensor relies on the fact that all biological material, such as proteins, cells, and DNA, have a dielectric constant that is higher than that of free space. Therefore, these materials all possess the intrinsic ability to slow down the speed of light that passes through them. The optical biosensors are designed to translate changes in the propagation speed of light through a medium that contains biological material into a quantifiable signal that is proportional to the amount of biological material that is captured on the sensor surface.

Different types of optical biosensors include but are not limited to ellipsometers, surface plasmon resonant (SPR) devices, imaging SPR devices, grating coupled imaging SPR devices, holographic biosensors, interference biosensors, Reflectometric Interference Spectroscopy (RIFS), Colorimetric Interference Biosensors, Difference Interferometers, Hartman Interferometers, Dual Polarization Interferometers (DPI), Waveguide sensor chips, Integrated Input Grating Coupler devices, Chirped Waveguide Grating devices, Photonic crystal devices, Guided Mode Resonant Filter devices based upon Wood's Anomalies, Trianglular Silver Particle Arrays. And further include devices that measure a variety of wavelengths of the electromagnetic spectrum including but not limited to visible, ultraviolet, near infrared, and infrared. The modes of operation include but are not limited to scattering, inelastic scattering, reflection, absorbance, Raman, transmittance, transverse electric wave, and transverse magnetic wave.

The surface plasmon resonance device is an optical biosensor that measures binding events of biomolecules at a metal surface by detecting changes in the local refractive index. In general, a high-throughput SPR instrument consists of an auto-sampling robot, a high resolution CCD (charge-coupled device) camera, and gold or silver-coated glass slide chips each with more than 4 array cells embedded in a plastic support platform. SPR technology exploits surface plasmons (special electromagnetic waves) that can be excited at certain metal interfaces, most notably silver and gold. When incident light is coupled with the metal interface at angles greater than the critical angle, the reflected light exhibits a sharp attenuation (SPR minimum) in reflectivity owing to the resonant transfer of energy from the incident light to a surface plasmon. Binding of biomolecules at the surface changes the local refractive index and results in a shift of the SPR minimum. By monitoring changes in the SPR signal, it is possible to measure binding activities at the surface in real time.

Since SPR measurements are based on refractive index changes, detection of an analyte is label free and direct. The analyte does not require any special characteristics or labels (radioactive or fluorescent) and can be detected directly, without the need for multistep detection protocols. Measurements can be performed in real time, allowing collection of kinetic data and thermodynamic data. Lastly, SPR is capable of detecting a multitude of analytes over a wide range of molecular weights and binding affinities. Thus, SPR technology is quite useful as a cellular response measurement system.

A CReMS for the measurement of complex impedance changes (delta Z, or dZ) of live patient cells is described in this embodiment where impedance (Z) is related to the ratio of voltage to current as described by Ohm's law (Z=V/I). For example, a constant voltage is applied to electrodes to which patient cells are attached, producing a current that at differential frequencies flows around, between cells and through cells. This CReMS is sensitive to the local ionic environment at the electrode interface with the cells and detects these changes as a function of voltage and current fluctuations. Physiologic changes of the cells as a result of normal function or activation thereof result in quantifiable changes to the flow of current around the electrodes and influence the magnitude and characteristics of the signal measured in such a CReMS.

In certain embodiments, the biosensor detects a change in global phenotype with event specificity. A global phenotype comprises one or more cellular response parameters selected from the group consisting of pH, cell adhesion, cell attachment pattern, cell proliferation, cell signaling, cell survival, cell density, cell size, cell shape, cell polarity, O₂, CO₂, glucose, cell cycle, anabolism, catabolism, small molecule synthesis and generation, turnover, and respiration, ATP, calcium, magnesium, and other charged ions, proteins, specific pathway member molecules, DNA and or RNA in various cellular compartments, genomics, and proteomics, post-translational modifications and mechanisms, levels of secondary messenger, cAMP, mRNA, RNAi, microRNAs and other RNA with physiologic function, and combinations thereof. With respect to event specificity, a cellular parameter is selected that reflects a change in a cell sample that is an expected change for that type of therapeutic and/or activator agent. For example, if a therapeutic agent is known to target a cytoskeletal element, a cell contacted with such an agent would be expected to show a change in cell adhesion in the presence of the agent.

In other embodiments, the change in attachment pattern is a change in cell adhesion. In some cases, the change in cell adhesion is indicated by a change in a refractive index or a change in impedance. In yet other embodiments, the change in attachment pattern is a change in basal morphology, a change in cell density, or a change in cell size or cell shape. In a specific embodiment, the change in basal morphology is a change in cell polarity. In embodiments, a decrease in cell signaling indicates a change in cytoskeletal organization.

In other embodiments, the methods of the disclosure provide for analysis of cell samples that are label free and that can be measured in real time. In one embodiment, the cell sample analyzed is a label free, viable, and not subject to any treatments to fix the cells. In another embodiment, the therapeutic and/or activator agents used in the methods and kits of the disclosure are also label free. To date label free methods have not been applied to determining therapeutic efficacy in effective ways.

Label free assays can reduce the time and cost of screening campaigns by reducing the time and misleading complications of label assays. Assays that can identify and quantify gene expression, gene mutation, and protein function are performed in formats that enable large-scale parallelism. Tens-of-thousands to millions of protein-protein or DNA-DNA interactions may be performed simultaneously more economically with label-free assays.

In contrast to the large variety of labeled methods, there are relatively few methods that allow detection of molecular interaction and even fewer still for cellular function without labels. Label-free detection removes experimental uncertainty created by the effect of the label on molecular folding of therapeutic and activator agents, blocking of active sites on cells, or the inability to find an appropriate label that functions equivalently for all molecules in an experiment that can be placed effectively within a cell. Label-free detection methods greatly simplify the time and effort required for assay development, while removing experimental artifacts from quenching, shelf life, and background interference.

Labels are a mainstay of biochemical and cell-based assays. Labels comprise the majority of all assay methods and have to overcome several problems, especially in the context of the study of complex dynamic activities in live human cells. Use of radioactive labels create large quantities of contaminated materials and must be used in specialized facilities with regulatory methods to prevent harm (at the cellular level) to those that use them. The excitation/emission efficiency of fluorophores is degraded by time and exposure to light, reducing the ability of the label to be accurate and precise, and requiring that assays be read once only in an end point manner so that temporal information cannot be obtained. All label-based assays require a significant amount of time to develop a process for attaching the label in a homogenous and uniform manner, determining that the label will be linearly quantitative, and will not interfere or affect the interaction or process being measured. The uniform application of labels in complex mixtures is complicated by the presence of all the molecules that are needed for the process to proceed naturally. Addition of the label only allows for visualization of that molecule function indirectly, not the entire system function directly (i.e. some extended assumptions may be necessary). Cellular activities are even more difficult to measure accurately with labels. A useful test must figure out how the label will get onto the right molecule, the right way, in the right location with respect to the cell, and be certain that the label is not disturbing the normal cellular processes.

Label-free detection generally involves the use of a transducer that is capable of directly measuring some physical property of a biological compound or bioentity such as a DNA molecule, peptide, protein, or cell. All biochemical molecules and cells have finite physical values for volume, mass, viscoelasticity, dielectric permittivity, heat capacity and conductivity that can be used to indicate their presence or absence, increase or decrease, and modification using a type of sensor. Additionally, living systems utilize molecules to provide energy and carry out their life processes, such as O₂/CO₂ consumption/generation, glucose production/consumption, ATP production/consumption that cause measurable changes such as pH in their environ over finite periods of time. The sensor functions as a transducer that can convert one of these physical properties into a quantifiable signal such as a current or voltage that can be measured.

In some cases, in order to use a transducer as a biosensor, the surface of the transducer must have the ability to selectively capture specific material such as a protein or specific cell type, while not allowing undesired material to attach. Selective detection capability is provided by building of a specific coating layer of chemical molecules on the surface of the transducer. The material that is attached to the sensor surface is referred to as the sensor coating while the detected material is called the analyte. Thus, in some cases, a biosensor is the combination of a transducer that can generate a measurable signal from material that attaches to the transducer, and a specific recognition surface coating containing a receptor ligand that can bind a targeted analyte from a test sample.

In certain embodiments, a coating is selected for a biosensor that is associated with a particular cellular component or pathway. For example, in those cases, where the cellular physiological parameter is change in cell adhesion, a coating is selected that provides for adhesion of the cells in the cell sample to the biosensor surface. In embodiments, the coating that enhances adhesion of the cells to the biosensor includes extracellular matrix, fibronectin, integrins and the like. In other embodiments, a coating is selected that binds to a particular cell type based on a cell surface marker. In some embodiments, such cell surface markers include GPCRs, such as lysophospholipid GPCRs (e.g., LPARs and S1PRs).

In other embodiments, the biosensor is coated with a biomolecular coating. CReMS surfaces contacting cells may contain a biomolecular coating prior to addition of cells, during addition of cells, or after addition of cells. The coating material may be synthetic, natural, animal derived, mammalian, or created by cells placed on the sensor. For example, a biomolecular coating can comprise an extracellular matrix component known to engage integrins, adherins, cadherins and other cellular adhesion molecules and cell surface proteins (e.g., fibronectin, laminin, vitronectin, collagens, IntercellularCAMs, VascularCAMs, MAdCAMs), or a derivative thereof, or can comprise a biochemical such as polylysine or polyornithine, which are polymeric molecules based on the naturally occurring biochemicals lysine and ornithine, polymeric molecules based on naturally occurring biochemicals such as amino acids can use isomers or enantiomers of the naturally-occurring biochemical, antibodies, fragments or peptide derivatives of antibodies, complement determining region (CDR), designed to attach specific cell surface proteins to the biosensor.

Methods for attaching viable cells to a microplate may include, for example, coating the sensor microplate surface with a reactive molecule having one end designed to interact with the surface of the biosensor, and another end that designed to react with functional groups on a peptide. For example, when using a gold-coated biosensor, the reactive molecule could include a sulfur atom or other chemical moiety designed to chemically interact with the biosensor surface. The other end of the molecule can specifically react with, for example, the amide or carboxy groups on a peptide.

Methods for attaching viable (e.g., primary cells) to a biosensor surface are also described in detail in PCT Application No. PCT/US2016/0579023, the entire contents of which is expressly incorporated herein by reference.

In another example, the biosensor surface can be coated with molecules that adhere through van der waals forces, hydrogen bonding, electrostatic attraction, hydrophobic interaction, or any combination of these such as one practiced in the art might use to apply proteins. An extracellular matrix (ECM) molecule can also be added to the first surface molecular coating. Humphries 2006 Integrin Ligands at a Glance. Journal of Cell Science 119 (19) p3901-03 describes adhesion molecules useful in this invention. Additional ECM molecules that can be used to contact specific cell adhesion molecules include those described in Table 1 of Takada et al., Genome Biology 8:215 (2007). This example is for integrins involved in cell-ECM and cell-cell adhesion. Many other adhesion molecules have been described with properties related to physiologic control and response (see Table 2 below).

TABLE 2
Ligand-binding specificities
of human integrins

| | |
|---|---|
| Integrins | ECM and cell-cell Ligands |
| α1β1 | Laminin, collagen |
| α2β1 | Laminin, collagen, thrombospondin, E-cadherin, tenascin |
| α3β1 | Laminin, thrombospondin, uPAR |
| α4β1 | Thrombospondin, MAdCAM-1, VCAM-1, fibronectin, |
| | osteopontin, ADAM, ICAM-4 |
| α5β1 | Fibronectin, osteopontin, fibrillin, thrombospondin, ADAM, |
| | COMP, L1 |
| α6β1 | Laminin, thrombospondin, ADAM, Cyr61 |
| α7β1 | Laminin |
| α8β1 | Tenascin, fibronectin, osteopontin, vitronectin, LAP-TGF-β, |
| | nephronectin, |
| α9β1 | Tenascin, VCAM-1, osteopontin, uPAR, plasmin, angiostatin, |
| | ADAM [25], VEGF-C, VEGF-D[26] |
| α10β1 | Laminin, collagen |
| α11β1 | Collagen |
| αVβ1 | LAP-TGF-β, fibronectin, osteopontin, L1 |
| αLβ2 | ICAM, ICAM-4 |
| αMβ2 | ICAM, iC3b, factor X, fibrinogen, ICAM-4, heparin |
| αXβ2 | ICAM, iC3b, fibrinogen, ICAM-4, heparin, collagen [27] |
| αDβ2 | ICAM, VCAM-1, fibrinogen, fibronectin, vitronectin, Cyr61, |
| | plasminogen |
| αIIbβ3 | Fibrinogen, thrombospondin,, fibronectin, vitronectin, vWF, |
| | Cyr61, ICAM-4, L1, CD40 ligand [28] |
| αVβ3 | Fibrinogen, vitronectin, vWF, thrombospondin, fibrillin, tenascin, |
| | PECAM-1, fibronectin, osteopontin, BSP, MFG-E8, ADAM-15, |
| | COMP, Cyr61, ICAM-4, MMP, FGF-2 [29], uPA [30], uPAR [31], |
| | L1, angiostatin [32], plasmin [33], cardiotoxin [34], LAP-TGF-β, |
| | Del-1 |
| α6β4 | Laminin |
| αVβ5 | Osteopontin, BSP, vitronectin, CCN3 [35], LAP-TGF-β |
| αVβ6 | LAP-TGF-β, fibronectin, osteopontin, ADAM |
| α4β7 | MAdCAM-1, VCAM-1, fibronectin, osteopontin |
| αEβ7 | E-cadherin |
| αVβ8 | LAP-TGF-β |

Additional coatings may include antibodies or other proteins known to have affinity for patient cell surface proteins so as to bring the patient cells into close proximity to the biosensor for the purpose of performing the methods described herein. It may also be beneficial to confirm that the patient cells are attached in the desired manner to the microplate. Specific biosensor coatings can additionally be used to enhance, improve, clarify, segregate, or detect specific cell signals from specific patient cell types and cell signaling responses to activation and therapeutics by linking the sensor coating to specific cellular pathways (see, e.g., Hynes, Integrins, Cell, 110:673-687 (2002)). A biosensor comprises an area to seed cells. For example, a biosensor can comprise a microtiter plate containing wells to seed cells. One or more cell samples can be seeded on a biosensor by physical adsorption to a surface in a distinct location. A biosensor can comprise 1, 10, 24, 48, 96, 384, or more distinct locations. A cell sample can comprise about 100 to about 100,000 individual cells or any cell number in between. An optimal cell sample depends on the size and nature of a distinct location on a biosensor. A cell sample can comprise about 5000 cells or less; about 10,000 cells or less; about 15,000 cells or less; about 20,000 cells or less; about 25,000 cells or less; or about 50,000 cells or less. A cell sample can comprise about 1000 to about 2500 cells; about 1000 to about 5000 cells; 5000 to about 10,000 cells; about 5000 to about 15,000 cells; about 5000 to about 25,000 cells; about 1000 to about 10,000 cells; about 1000 to about 50,000 cells; and about 5000 to about 50,000 cells. In certain embodiments, a change in a cellular response or physiological parameter is measured over a defined period of time. In other embodiments, the defined period of time is the amount of time that it takes for the control cells to reach a steady state in which a change in the output of the physiological parameter varies by 20% or less. In other embodiments, the change is observed in cells in 1 hour or less. In other embodiments, the change is observed in cells for at least 1 min. to about 60 min. and every minute in between. In other embodiments, the change in cell response is measured from about 10 minutes to about one week or 200 hours. In other embodiments, when a therapeutic agent is targeted to a cellular pathway, the cellular response is measured from about 10 minutes to about 5 hours, about 10 minutes to about 4 hours, about 10 minutes to about 3 hours, about 10 minutes to about 2 hours, about 10 minutes to about 1 hour, or about 10 minutes to about 30 minutes or any time point in between. In other embodiments, when a therapeutic agent affects cell proliferation or cell killing or cellular resistance, the cellular response is measured from about 1 hour to about 200 hours. In yet other embodiments, a combination of responses (otherwise described as a full temporal pattern) between 1 minute and 200 hours is used to determine therapeutic effect of a compound on cells and the cells ability to develop resistance. This timeframe encompasses the important process of short-term pathway signaling, dynamic reprogramming and longer term cellular responses important in assessing a probable response and maintenance thereof in a patient.

Once cells of a particular subject have been seeded on a biosensor, baseline measurements can be determined. Baseline measurements can be taken on the same cell sample, or a control cell sample. The control sample can comprise healthy cells or diseased cells from the same patient and/or same tissue. The control sample can comprise diseased cells that do not receive GPCR agonist, RAS node or RTK inhibitor, or RAS node or RTK targeted therapeutic agent. A control sample can comprise disease cells known to respond to the GPCR agonist. In other embodiments, the control sample comprises disease cells known not to respond to the GPCR agonist. The control sample may include application of an agonist to healthy or diseased cells of a particular patient, designed to elicit a standardized response relating to cell health, cell metabolism, or cell pathway activity.

The control would be determined for each disease and/or drug type. In one embodiment, this involves a comparison against a healthy cell control from the same patient or comparison against a result for a pool of non-diseased patients (e.g. a normal reference range). For example, with cell killing drugs, the method will show benefit of killing disease cells over healthy cells to achieve a significant therapeutic index. Other embodiments include the use of pathway tools to determine pathway function and control by the drug. For targeted therapeutics, the tools can be activator agents (e.g., activator agents), bioreagents or small molecules which are used as controls to perturb a pathway and determine a targeted drug's ability to disrupt the activation. In yet other embodiments, the physiologic effect of a drug on a cell is measured without exogenous perturbation by an activator agent noting, for example, the temporal pattern or rate of oxygen consumption, the rate or temporal pattern of acidification, ion flux, or metabolite turnover.

In a particular embodiment, the biosensor signal is measured over a continuous time course. There are distinctive patterns on the time vs. biosensor signal plot that are indicative of a patient cell response to drug treatment. Evaluation of these patterns is useful to identify the presence of an efficacious event. A time course or constantly changing measurement of live and fully functional cells is more beneficial than the current practice used in typical whole cell assays that only represent a point in time. The methods described herein measure dynamic systems as they would occur in a patient and represent the most accurate means of determining patient response. In the case of pathway responses, recording of a complete time course or temporal pattern is superior in ability to support more complex analysis and obviates selecting the optimum time point for a single measurement.

Comparison against controls could occur at a temporal maxima, minima, or as differences between maximal signal-minimal signal, or by comparing integrated areas under a curve (AUC) for a time course plot or other non-linear comparisons (e.g. summation of difference vectors) of the test well against positive or negative control wells or comparisons of perturbed and unperturbed wells for the same patient viable diseased cells. Additional analyses supported only by measuring with a biosensor are time to reach maxima/minima, and other derivatives of the temporal time course. In the case of longer term responses, the time of comparison may be of a specific time point after a few days or a week of treatment or multiple applications of drug. The longer time course may also compare changes in slope or compare second derivatives of the time versus biosensor signal plot at the beginning, middle or end of a week of drug treatment. Significant changes compared to control may include absolute drop in biosensor signal related to curtailment of cellular metabolism. Alternatively, the drop may be followed by an increase that could indicate development of resistance to the drug during the assay. Additionally, non-linear Euclidean analyses could be used to produce a measure of total differences between controls and patient samples over a complete time-course. This too would be significant with respect to predicting the outcome for a patient.

In certain embodiments, the output of a biosensor over a defined period of time is represented as a cell index. The cell index is the change in impedance from a test starting point. Cell Index is defined as a measurement of impedance and can be applied in one instance of the present invention by measuring at a fixed electrical frequency of, for example, 10 kHz and fixed voltage.

And calculated by the equation Cell Indexᵢ=(Rₜₙ−Rₜ₀)/F

Where:

i=1, 2, or 3 time point

F=15 ohm in one example when the instrument is operated at 10 kHz frequency

Rₜ₀ is the background resistance measured at time point TO.

Rₜₙ is the resistance measured at a time point Tn following cell addition, cell physiologic change, or cell perturbation.

Cell index is a dimensionless parameter derived as a relative change in measured electrical impedance to represent cell status. When cells are not present or are not well-adhered on the electrodes, the CI is zero. Under the same physiological conditions, when more cells are attached on the electrodes, the CI values are larger. CI is therefore a quantitative measure of cell number present in a well. Additionally, change in a cell physiological status, including cell morphology, changes in basal, stable, or quiescent condition, cell adhesion, or cell viability will lead to a changes in CI.

The cell index is a quantitative measure of the presence, density, attachment or changes thereof based upon a starting point or baseline impedance measurement. The baseline starting point impedance is a physical observable characteristic and an indication of the health, viability, and physiologic status of a cell prior to any treatment with drug or other activator. The baseline starting point can be used as a qualitative control for the CELx test. Addition of drug or activator causes the impedance to change in temporal patterns reflective of the specificity of the cellular physiologic change experienced by the cell. Changes in a cell physiological status, for example cell morphology, cell number, cell density, cell adhesion, or cell viability will lead to a change in the cell index.

Physiologic response parameters can additionally include cell cycle analysis and can be measured using any number of chemical biosensors such as fluorescent dyes conjugated or unconjugated or other colorimetric changes in patient cells associated with functional and dysfunctional pathways. For example, changes in cell cycle for a population of cells using an unconjugated dye can be quantified with propidium iodide or similar dyes known to intercalate into DNA and correlate with cell cycling through G0, G1, S, G2, Gm phases of growth and replication by assessing changes in the amount of DNA. With one dye type, propidium iodide, the fluorescence of cells in the G2/M phase will be twice as high as that of cells in the G0/G1 phase. Propidium iodide can also intercalate into RNA and often ribonucleases are used to differentiate fluorescence signal from DNA compared to RNA. Examples also include dyes specific for particular proteins linked to cell cycle check points and provide additional cell cycle status measurement. Common instruments useful for performing these measurements but not limited to those listed here are fluorescence microscopy, confocal laser scanning microscopy, flow cytometry, fluorometry, fluorescence polarization, homogenous time resolved fluorescence, and fluorescence activated cell sorting (FACS).

Unconjugated dyes can be utilized with the present invention as a chemical biosensor of physiologic status of a cell or pathway while measuring metabolic parameters such as anabolism, catabolism, small molecule synthesis and generation, turnover, and respiration. A well-known cell physiologic response, named the Warburg Effect, describes the shift from oxidative phosphorylation to lactate production for energy generation in tumor and other diseased cells, and key signaling pathways, oncogenes and tumor suppressors can be measured by any of the chemical biosensor methods described here or by opto-electronic biosensors. Cellular oxygen consumption or respiration and glycolysis in cellular responses produces protons and causes rapid, easily measurable changes to the concentrations of dissolved oxygen and free protons or acidity.

An additional but not limiting example of a physiologic response parameter utilizing a chemical biosensor is the amount of ATP being utilized by cells in culture based on quantitation of the ATP present (Ex. CellTiterGlo and similar luciferase driven assays), an indicator of metabolically active and inactive cellular function.

Calcium, magnesium, and other charged ions that are important for biomolecular folding and function are in flux due to physiologic response. These too can be measured by chemical biosensors such as Cal-520, Oregon Green BAPTA-1, fura-2, indo-1, fluo-3, fluo-4, Calcium Green-1, and other EGTA or EDTA-like chemistries for specific ion complexation and measurement. These physiologic response parameters can be measured using many types of unconjugated reactive or binding dyes or other electronic or spectroscopic means. Many of these methods can be arranged so as to be non-destructive to the cells allowing the physiologic function of the same cell population to be continuously measured repeatedly over time.

Conjugated dyes such as those attached to natural cell protein binding ligands or attached to immunoparticles (antibodies or fragments of antibodies or high specificity high affinity synthetic molecules such as aptamers), or nucleic acid polymer hybridization probes can be used to measure physiologic response parameters related to proteins, specific pathway member molecules, DNA and or RNA in various cellular compartments, genomics, and proteomics, and are able to measure specific post-translational modifications and mechanisms. The post-translational modification and epigenetic means of cellular control can involve regulation by a multitude of enzymes performing pathway functions that include but are not limited to ribozymes, kinases, phosphatases, ubiquitinases, deubiquitinases, methylases, demethylases, and proteases. Examples of these molecules used for staining formalin fixed paraffin mounted samples of dead cells can be found in the DAKO Immunohistochemical Staining Methods Education Guide—Sixth Edition or at Cell Signaling Technology tutorials and application guides http://www.cellsignal.com/common/content/content.jsp?id=tutorials-and-application-guides. These two examples may be even more useful with the present invention for measuring live cell response. Common instruments useful for performing this measurement but not limited to these methods are fluorescence microscopy, confocal laser scanning microscopy, flow cytometry, fluorometry, homogenous time resolved fluorescence, fluorescence polarization, and fluorescence activated cell sorting (FACS).

Combinations of conjugated and non-conjugated dyes can also be employed by the present invention to measure physiologic response of cells. Following activation, one type of receptor responsible for controlling physiologic response are GPCRs. They transmit information and control cells via two signaling pathways: changes in the level of secondary messenger cAMP, or changes in the level of intracellular Ca2+, which is liberated by secondary messenger inositol (1,4,5) triphosphate (IP3). cAMP detection for example can be based on a competitive immunoassay using cryptate-labeled anti-cAMP antibody (or other immunocapture molecule) and d2-labeled cAMP that competes with cellular cAMP for the GPCR reaction and subsequent antibody binding. The specific signal (i.e. energy transfer) is inversely proportional to the concentration of cAMP in the standard or sample.

Measurement of physiologic response by quantifying mRNA, RNAi, microRNAs and other RNA with physiologic function can be a very sensitive method employed with the practice of the present invention for determining activation of a cellular change at the transcription level. RNA can be quantified for example but not limited to these listed here by using rtPCR, qPCR, selective sequence probing, selective sequence capture, and sequence hybridization methods that all employ chemical sensors.

Immuno-capture and hybridization methods include those using bead based methods such as Luminex or fiber optic tip technologies such as Illumina or protein, DNA, RNA, or other hybridization microarray technologies where the specific capture reagent is immobilized onto a solid surface that is used to fish out, isolate, and accurately measure the physiologic response molecule(s) from the cells. These methods offer the benefit of measuring a multitude of response parameters in a single experiment.

A change in a cellular response or physiological parameter is determined by comparison to a baseline measurement. The change in cellular parameter or physiological response depends on the type of CReMS. For example, if the change in cellular response is determined optically, physically observable changes could be measured for example as a function of optical density at spectral wavelengths for chemical absorbance or transmittance, changes in a surface plasmon measurement device, or changes detected by photonic crystal devices. If the change in cellular parameter or physiological response is determined electrically, physically observable changes could be measured for example using milli or micro impedance changes of cells adhered to electrodes. Changes in pH, glucose, carbon dioxide, or ions, could be measured electronically using ion selective field effect transistors (ISFET).

In other embodiments, a rate of change is determined by a method measuring a CReMS response for a period of time required to determine a difference in cellular physiologic response to a therapeutic. The rate of change is described by various interpretation of the time course data and can be expressed as a rate or further derivative function of the rate including acceleration of the rate.

Tests that measure a physiological condition of a patient can derive one or more cutoff values above which and below which the patient is predicted to experience different clinical outcomes. In embodiments, one or more cutoff values for determining a change in cellular response is determined by a method comprising: determining a standard deviation, a signal to noise ratio, a standard error, analysis of variance, or other statistical test values known by those practiced in the art for determining appropriate confidence intervals for statistical significance of a set of samples from known responding cell samples and from a set of samples from known nonresponding patients; and determining the difference between the two and setting the cutoff value between the confidence intervals for both groups. An additional embodiment utilizes a cutoff determined from a normal reference range defined by CReMS response from patients known not to be diseased. In this embodiment, a single patient disease material test result is reported by comparing perturbed and unperturbed viable cancer cell results as described further elsewhere in the present invention to the non-diseased reference range interval.

A normal (healthy) reference range test result establishes what “normal” pathway activity is by conducting a study using normal tissue from healthy subjects. The test result then assesses whether any patients who are not expected to have the diseased pathway (e.g. biomarker negative patients) do in fact have abnormal pathway activity when compared to the values derived from the normal reference interval study. Results of the present invention are also compared for the abnormal measurements observed from biomarker-negative patients against the measurements made from those subjects who are currently diagnosed with the disease (biomarker positive patients) to see if the patients who are biomarker negative have pathway activity that is both abnormal and comparable to the pathway activity of biomarker positive patients. Those patients who have abnormal pathway activity that is comparable to the pathway activity of patients currently receiving and benefiting from therapies intended to disrupt the pathway activity would thus be diagnosed as having the pathway disease and should thus be treated with the drug that targets the biomarker (e.g., RAS node or RTK inhibitor) in order to disrupt the pathway activity (e.g., GPCR signaling pathway activity).

The present invention thus enables physicians to create more precise means of diagnosing a disease based on the functional activity of a diseased pathway. This is in contrast to the approach taken with when a single biomarker is measured that relies on a correlative, not a causative model. Current biomarker approaches can result in a high percentage of false negative and false positive results. The present invention will reduce the percentage of false results.

Preferred embodiments include 80-90% confidence intervals, more preferred embodiments include >90% confidence intervals and most preferred embodiments include >95% or >99% confidence intervals.

In some embodiments, a cutoff value is validated by determining the status of blinded known samples as responders or non-responders using a cutoff value and unblinding the sample and determining the accuracy of predicting the status of the sample. In the case of a single cutoff value, output values that fall below the cutoff value or are closer to the output values for the known responders indicate the patient sample is exhibiting responsiveness to the therapeutic agent. If the output values are at or above the cutoff output value or are closer to the output values for the known non-responders output value, the cell sample is identified as a non-responder to the therapeutic agent. In some embodiments, an output value of the biosensor at a defined period of time is classified as no response, weakly responsive or responsive.

In preferred embodiments, a cutoff value is validated by determining the status of blinded known samples as having the disease pathway response (e.g. cancer patients with abnormal GPCR signaling) or not having the disease pathway response (e.g. cancer patients with normal GPCR signaling) using a cutoff value and unblinding the samples and determining the accuracy of predicting the status of the sample. In the case of a single cutoff value, output values that fall below the cutoff value or are closer to the output values of the samples not having the disease pathway response indicate the patient sample is exhibiting no pathway disease. If the output values are at or above the cutoff value or are closer to the output values for the known diseased pathway samples values, the cell sample is identified as diseased pathway present. In some embodiments, an output value of the biosensor at a defined period of time is classified as pathway disease not present, pathway disease inconclusive, or pathway disease present.

An output value at a defined period of time is selected in order to classify the output into the categories. In other embodiments, the defined period of time is the end point of the time period for which the cells have been continuously monitored in the biosensor. In other embodiments, the time period is at least 60 minutes, 120 minutes, 180 minutes, 240 minutes, 300 minutes, 10 hours, 24 hours, 60 hours, or 120 hours. In preferred embodiments the output at a defined period of time is between 30 minutes and 10 hours. In more preferred embodiments, the output at a defined period of time is between 180 minutes and 600 minutes or is 240 minutes.

In other embodiments, the cancer cells of a randomly selected population of cancer patients are tested using the methods described herein. It is expected that the cancer cells obtained from a population of cancer patients will exhibit a wide range of signaling activity levels or output values. To characterize the population distribution of signaling activity levels or output values, a finite mixture model analysis, or other statistical analysis of the individual output values using statistical analysis software (e.g. mixtools, an R package for analyzing finite mixture models) is performed. If two or more groups are identified within the population of cancer patients analyzed, a cut-off value between the mean output value of one group and the mean output value of the second or other groups is selected.

In embodiments, an output value classified as no response is indicated by an output value that differs from the output value of the baseline prior to administration of a therapeutic agent or a control cell not treated with the therapeutic agent by no more than at least 20% or less, 15% or less, 10% or less, or 5% or less.

In other embodiments, an output value classified as weakly responsive is indicated by an output value that differs from the output value of the baseline prior to administration of a therapeutic agent or a control cell not treated with the therapeutic agent by at least 50% or less and greater than 5%. In other embodiments, an output value percentage classified as responsive is indicated by an output value that differs from the baseline prior to administration of a therapeutic agent or a control cell not treated with the therapeutic agent by at least greater than 50%. In embodiments, the control sample is a sample of the disease cells from the same subject and not treated with the therapeutic agent.

A further aspect of the methods described herein involves developing an algorithm that can be used to predict the efficacy of a therapeutic agent (e.g., an inhibitor of a RAS node or RTK signaling) in an individual subject. The algorithm incorporates the values derived using the methods described herein, in combination with values assigned to one or more patient characteristics that define an aspect of an individual subject's health. Patient characteristics can include, but are not limited to, the presence of metastases, the location of metastases, nodal status, disease free interval from initial diagnosis of cancer to diagnosis of metastases, receipt of adjuvant chemotherapy, receipt of other drug therapies, receipt of radiation therapy, dominant site of disease, tumor mass size, body-mass index, number of tender joints, number of swollen joints, pain, disability index, physician global assessment, patient global assessment, Bath Ankylosing Spondylitis Functional Index, Bath Ankylosing Spondylitis Disease Activity Index, Bath Ankylosing Spondylitis Metrology Index, C-Reactive Protein, total back pain, inflammation, genetic status, history of other illnesses, other vital health statistics status, and any combinations thereof. The algorithm that incorporates these values would weight these values according to their correlation to disease progression in a population of patients with the disease that the therapeutic agent is intended to treat. Disease characteristics that did not demonstrate any correlation with differential response would not be included in the algorithm.

In one embodiment, a numerical value representing the patient characteristics can be derived from a regression analysis of the test results (i.e., values derived from the methods of determining responsiveness to a targeted therapeutic agent, an agonist of GPCR signaling, a combination of a targeted therapeutic agent and agonist, etc., as described herein), the patient characteristics, and the clinical outcome of a group of patients studied. In one example, optimization of an algorithm using the test results in combination with variables based on patient characteristics data can be performed by dividing the test output values into 10 intervals based on 9 equally spaced cut-points of width 0.10 beginning with 0.10. For each cut-point, a Cox regression can be run using an indicator variable which takes on the value “one” if a subject has an algorithm value less than or equal to the cut-point and “zero” otherwise. The hazard ratio, being the comparison of those at or below the cut-off, versus those above the cut-off, will be determined for each cut-point. The value of the cut-point that minimizes the estimated hazard ratio is then selected.

For example, it may be found that when a patient's total tumor mass is above a certain value, their responsiveness to a drug, as determined by the methods described herein, will not be sufficient to prolong the patient's potential progression free period beyond the median result found for those patients not responsive to the drug. In the case when a test result indicates that the drug is functional in the patient, and that they would otherwise be expected to benefit from it, the algorithm including the patient characteristics variables would report that the result is indeterminate since the tumor mass variable offsets the test result value.

Another aspect of the methods described herein provides a method for determining a cut-off value for a test that identifies patients likely or unlikely to respond to a targeted therapeutic agent. This method involves a) selecting a group of patients, each of whom has the same disease and is prescribed the same therapeutic, b) using the methods described herein to derive a test value for each subject within a group of patients, c) observing the health status of each member of the group of patients tested over a period of time sufficient for a significant percentage of the total patients tested to reach a predefined clinical endpoint and record the length of time required for each of the patients to reach, if they did, the predefined clinical endpoint, d) identifying two or more candidate cut-off values that are equidistant in value to the other, wherein each candidate cut-off value represents a value below which a patient is predicted to respond or not respond and above which a patient is predicted to respond in opposite manner of those whose scores fell below the cut-off value, e) using a statistical method to analyze the difference between the clinical endpoint periods for patients whose test value was at or below the cut-off and the clinical endpoint periods for those patients whose test value was above the cut-off, and f) selecting the cut-off value that results in the greatest percentage of patients who are predicted not to respond to the therapy amongst the group of candidate cut-off values that indicates there is a statistically significant difference between the group of patients above and below the cut-off value.

Using the methods described herein, it is possible to derive a numeric test result value for an individual subject that can be compared to the test value derived from other individuals with the same disease whose cells were tested with the same therapeutic. This makes it possible to predict the efficacy of a therapeutic on an individual subject by: a) recording the test result values for a group individual subjects who have the same disease and were tested with the same therapeutic, b) compiling those values into a list, c) ordering the list on the basis of test results values for the individual subjects tested on the basis of each individual subject's absolute numeric test value, and d) determining the percentile rank of an individual subject's test value, wherein the percentile rank of an individual subject's test value is predictive of the efficacy of the therapeutic agent for the disease in the individual subject.

Another embodiment includes analyzing the results obtained from a clinical trial testing the efficacy of the same therapy to estimate the percentile ranking of a particular result and then identifying the percentile rank for an individual subject's test value, and identifying the clinical trial end point result that corresponds to the same percentile ranking, wherein the clinical trial end point result at the same percentile ranking as the individual subject's test value is predictive of the clinical result an individual subject is likely to obtain from the therapeutic agent for the disease. The clinical trial end points can include, for example, time-to-progression period, progression-free survival period, overall survival period, objective response period, ACR response, change in Total Sharp Score, erosion score, and Joint Space Narrowing, clinical response, pain, disability index, clinical remission, body-surface area involvement, physicians global assessment, and psoriasis area and severity index.

Another embodiment includes a method to determine the statistical correlation between the test result values derived from the methods described herein and the clinical outcome for an individual who received the therapeutic that was tested. This method comprises: a) selecting a group of patients, each of whom has the same disease and is prescribed the same therapeutic, b) using the methods described herein to derive a test result value for an individual, c) compiling a list of test result values for each subject within a group of patients who have the same disease and were tested with the same therapeutic, d) observing the health status of each member of the group of patients tested over a period of time sufficient for a significant percentage of the total patients tested to reach a predefined clinical endpoint, e) recording the length of time required for each of the patients to reach, if they did, the predefined clinical endpoint, and f) analyzing the end-point data (e.g. time-to-progression period, progression-free survival, ACR response) in such a manner that the statistical relationship between the end point result and the test value is determined.

By way of example, once the results from a clinical trial are available, the determination of an estimate of the cut-off value—“C*”—proceeds as follows. Assuming that a Cox regression test indicates that the test value is predictive of a patient outcome, such as time-to-progression (TTP), the test values will be divided into 10 intervals based on 9 equally spaced cut-points of width 0.10 beginning with 0.10. For each cut-point, a Cox regression will be run using an indicator variable which takes on the value “one” if a subject has an assay value less than or equal to the cut-point, and “zero” otherwise. The hazard ratio, being the comparison of those at or below the cut-off versus those above the cut-off, will be determined for each cut-point. The value of the cut-point that maximizes the estimated hazard ratio will be selected for use in the subsequent pivotal phase of the study. For the final analysis, a Cox proportional hazard regression can be run with an indicator variable (below the cut-point versus above the cut-point). The final analysis can also include other putative predictive patient characteristic variables of TTP.

When a therapeutic agent is a targeted therapeutic agent that binds to a cell surface receptor, the change in cellular responsiveness is measured in the absence or presence of an activator agent or perturbant that binds to the receptor. In some embodiments, the therapeutic agent is administered to the cell sample before, at the same time or after the activator or perturbant. In some embodiments, the activator agent or perturbant is label free. A therapeutic agent is selected that inhibits the cellular responsiveness to the activator agent or perturbant as compared to baseline measurement and optionally, as compared to other therapeutic agents, regardless of the density of the cell surface receptors. In some embodiments, a therapeutic agent is selected that inhibits the action of the activator agent or perturbant independent of the density of cell receptors.

The change in the physiological parameter can be an increase or a decrease in the parameter as compared to baseline or healthy or unperturbed cell control. The changes could represent full agonism, superagonism, irreversible agonism, selective agonism, co-agonism, inverse agonism, or partial limiting agonism, reversible and irreversible antagonism, competitive antagonism, non-competitive antagonism, or un-competitive antagonism. The changes can occur sooner, later or not at all as compared to an appropriate control. The changes could be selected to occur for a longer or shorter period of time. Changes could be selected that are reversible or irreversible.

For example, a therapeutic agent that results in a decrease in cell signaling would be selected for treatment of an autoimmune condition. Peripheral blood cells that respond to an agent that inhibits the action of a cytokine show a decrease in cell signaling. In another example, for disease cells responsive to an anticancer agent, such as a humanized antibody targeted to a receptor like Her2, the disease cells would show a significant reduction in EGF family pathway signaling. In other cases, for disease cells responsive to an anti-angiogenic agent, the disease cells would show a reduction in VEGF pathway signaling or reduction in proliferative ability. The CReMS response or physically observable characteristic measured for each type of agent is dependent upon the intended physiological response the drug was designed to illicit and can be as specific or general as needed. The key is the use of the CReMS for physiological measurement of a live cell for a period of time to test the response the drug was intended to alter.

A particular therapeutic agent or agents can be administered to the diseased cells, and optionally, healthy cells to determine the effectiveness of the particular therapeutic or therapeutics. Diseased cells and/or healthy cells can also be untreated so as to compare the effect of the therapeutic or therapeutics on treated and untreated diseased and/or healthy cells. A single therapeutic can be administered to determine how a subject will respond to the therapeutic treatment. In another embodiment, a panel of different therapeutics can be administered to cells of a particular subject.

In certain embodiments, a cutoff value for efficacy of a therapeutic agent (e.g., a RAS node or RTK targeted therapeutic agent) to inhibit activation of a cellular pathway (e.g., a GPCR signaling pathway, such as a lysophospholipid GPCR signaling pathway) is determined in one embodiment by adding the drug and measuring the physiologic response. In another embodiment, the pathway is perturbed with and without drug pre-treatment. Changes to the physiologic baseline signal or reductions of the activation signal by the drug at the 85% confidence interval or ideally greater than the 90% confidence interval or more ideally greater than the 95% or 99% confidence interval are deemed efficacious. In embodiments, a cutoff value for efficacy of a therapeutic agent that inhibits cell proliferation or enhances cell killing is determined by recording the physiologic response over time. Reductions to the physiologic baseline signal or deviation from the temporal pattern as compared to non-treated or healthy cells or a combination thereof by the drug at the 85% confidence interval or ideally greater than the 90% confidence interval or more ideally greater than the 95% or 99% confidence interval are deemed efficacious.

The sensitivity and specificity of the therapeutic agent for treating the disease of an individual subject is determined by comparing the cellular physiologic pathway response as measured by the CReMS to determine that the drug is working as it was designed on a specific target and determining that a cutoff value for efficacy has been attained.

In some embodiments, the activator agent (e.g., a GPCR agonist) and/or the therapeutic agent (e.g., RAS node or RTK targeted therapeutic agent) are titrated in order to obtain the Hill Slope, EC₅₀ or IC₅₀ value for either agent. The data obtained from the activating agent titration and/or the therapeutic agent titration may be used to assess the potency (what concentration achieves one half maximal effect) and or efficacy (maximum achievable effect) of either agent.

In one embodiment, the method for determining therapeutic efficacy of an agent (e.g., a RAS node or RTK targeted therapeutic agent) for a disease in an individual subject comprises: administering the agent to at least one isolated disease cell sample from the individual subject in a cellular response measurement system (CReMS); and determining whether a change in a cellular response parameter of the cell sample to the agent occurs as compared to a baseline measurement, wherein the change in cellular response indicates that the agent has therapeutic efficacy for the disease in the individual subject. In embodiments, a method further comprises administering to at least one isolated disease cell sample from the individual subject in a cellular response measurement system an agonist that activates a GPCR signaling pathway (e.g., an agonist of a lysophospholipid GPCR) before or after administering the therapeutic agent.

The test can measure the effectiveness of a drug in a range of concentrations from below 1 nM to greater than 100 uM generally with less than 20% standard deviation and optimally with less than 5% standard deviation. The compound test range will correspond to dosing levels as defined on a drug packaging label known as the maximum tolerated dose. Unlike most tests that cannot ascertain the number of live cells in the actual set of cells in the test, this test is only working with the live cells as determined in a quality control and baseline physiologic determination step at the beginning of the test. The result of this feature reduces the variance of the test result. The test can be conducted using a temperature, oxygen, humidity, and carbon dioxide range generally acceptable for cell viability commonly known to those practiced in the art. In some cases, a preferred temperature range is between 25° C.-40° C. In other cases, the temperature may be optimized further to +0.5° C. within this range for specific perturbations and maintained using standard temperature controlled incubator cabinets.

Methods of the invention include administering candidate therapeutics to a subject's cells to determine safety and to determine therapeutic effectiveness. Additionally, administration of a candidate therapeutic to a subject's diseased cells may be used as a method of selecting the proper patient population for a phase II or III clinical trial. Methods of the invention include testing diseased cells against known therapeutic combinations. Additionally, methods of the invention include testing known and candidate therapeutics.

Methods of the invention also including administering combinations of therapeutic agents to determine if a particular combination of agents produces a more effective result (i.e., amelioration or cure of disease symptoms). A combination of therapeutic agents is two or more therapeutic agents administered to the same cell sample. In an embodiment of the invention, the combination of therapeutic agents is administered to a cell sample concurrently. In an embodiment, at least one therapeutic agent is administered to the cell sample at a time different than the administration of the other at least one therapeutic agent of the combination.

After administration of therapeutic agents to a cell sample, real time data can be collected on multiple aspects of the cell sample. For instance, pH and temperature can be measured. Additionally, other factors, such as “cell death factors”, can be determined. A cell death factor as determined by a CReMS can be a change in a physicochemical property as measured by the CReMS. For instance, cancer cells will attach to a surface and provide a baseline reading for a refractive index. Administration of a therapeutic agent that promotes cancer cell death would cause a change in the refractive index since the cancer cells in a sample would round up and detach from a surface. This could be measured by an optical biosensor utilizing surface plasmon resonance in a continuous real-time manner.

In certain embodiments, the methods involve determining an optimal dose range for a particular therapeutic. Determination of a dose range allows for proper design of clinical trials and/or allows the physician to balance efficacy with detrimental side effects. In some embodiments, a method comprises administering a range of doses of a therapeutic agent to separate samples of diseased cells from the same patient, and determining the dose range that results in a change in a physiological parameter of the cells as described herein as compared to baseline and/or healthy control cells.

Once any of the methods described herein are used to determine whether an individual subject's disease cells respond to one or more therapeutic agents (e.g., a RAS node or RTK targeted therapeutic agent), for example, upon stimulation with an agonist of a GPCR signaling pathway (e.g., a lysophospholipid GPCR signaling pathway), the results are communicated to a health care worker to allow for selection of a therapeutic agent for treatment of the subject. In some embodiments, the methods further comprise administering the selected therapeutic agent to the subject.

Measuring the signaling pathway activity can detect the presence of abnormalities consistent with the disease. To accomplish this, a platform has been developed that leverages the intimate connection between cellular signaling pathway operation and cell adhesion processes. Interaction of transmembrane cell adhesion receptors, such as integrins, cadherins, Ig CAMs, and selectins, with their cognate binding sites in the extracellular matrix or on other cells, has demonstrated connection to multiple cellular signaling processes. The adhesion connections communicate through organized membrane-proximal cytoskeletal structures that are directly linked to intracellular signaling cascades. This makes it possible to affect specific adhesion molecules via specific cellular pathways upon application of pathway activators.

To measure how activation of a cellular pathway effects cell adhesion, a device is used that measures complex impedance changes of viable patient cells attached to specific extracellular matrix (ECM) materials coating a microelectrode. Known as cellular impedance biosensors, these devices are comprised of a standard microplate with thin gold electrodes covering the bottom of each well. Wells employed with a selective extracellular matrix attach viable cells in a specific manner to the microplate well electrodes. The presence of viable cells on top of the well electrodes affects the local ionic environment at the electrode/cell interface, leading to an increase in electrode impedance. When cells are perturbed or stimulated to change their function, the accompanying changes in cell adhesion thus alter the impedance. Specificity of the adhesion response can be determined by the application of specific ECM or tool compounds or drugs known to act at various points in the pathway. Impedance results are further supported by immunodetection of specific proteome changes at time points indicated by the impedance temporal pattern. Systems are capable of detecting adhesion changes in the sub-nanometer to micrometer range and generate data for categorizing various pathway pharmacologies on live cells. The amount of impedance measured, referred to as a cell attachment signal (CAS), expressed in ohms, can be used to monitor cell viability, adhesion, and signaling pathway activation. Data generated is impedance versus time.

Therefore, in a diagnostic test of the invention, the analyte is the cell attachment signal (CAS) that viable patient cells generate, alone or in the presence of cell activators, when placed in the well of a microplate and analyzed with a biosensor such as an impedance biosensor. For every test, the CAS is measured and analyzed for two groups of patient cell samples.

1) Patient cells only (C)

2) Patient cells+activator pathway factor(s) (CF)

3) Patient cells+activator pathway factor(s)+confirming agent (CCF)

In some embodiments, C corresponds to patient cells only, CF corresponds to patient cells+an agonist of GPCR signaling, such as an agonist of a lysophospholipid GPCR (e.g., LPA, S1P), and CCF corresponds to patient cells+an agonist of GPCR signaling+an inhibitor of a RAS node or RTK signaling (e.g., a RAS node or RTK targeted therapeutic).

To detect whether the signaling pathway is functioning normally or abnormally, the signaling pathway in a patient's diseased cells are perturbed with an agonist, e.g., an agonist of GPCR signaling pathway, and a confirming agent (e.g., an inhibitor of a RAS node or RTK signaling) and the resulting activity is compared to the effect the agonist and/or the confirming agent has on a cut-off value. The cut-off value can be derived from a study involving analysis of the signaling pathway activity of a sample set of healthy cells obtained from subjects who do not have cancer. The assay measurand reflects the change in CAS between the CF and C cells in a patient's diseased cells and the change in CAS between the CF and CCF diseased cells. If the signaling pathways are abnormal, the CAS change between the CF and C diseased cells and/or the CAS change between the CF and CCF diseased cells will be above a cut-off value. The cut-off value is typically above the upper limit of the normal reference interval for the pathway activity of interest. In a simplified embodiment, the measurement of the CAS of the CF and/or CCF samples after the point of activation compared to the CAS at the point in time immediately before activation with the pathway factor or confirming agent may also be a useful measure.

The diagnostic assays of the invention can be used in essentially any clinical situation, in particular those in which currently a genetic or protein biomarker is used as an indicator of disease and thus as an indicator for therapeutic decision-making. In accordance with the methods of the invention, the approach described herein can be used to examine the activity of a RAS node or RTK that is affected by an agonist of a GPCR signaling pathway to determine whether a RAS node or RTK targeted therapeutic agent should be prescribed to a patient, regardless of whether they exhibit a positive result using the standard biomarker assay.

Methods of analyzing the continuous measurements to determine whether a change in a physiological response parameter occurs in the cellular sample are described herein (e.g., magnitude of response (positive or negative), time to max or min, slope of time vs. magnitude at any point of the response timeline, etc.). These and other methods of non-linear analysis can be used to determine whether a change in a physiological response parameter occurs in the presence of an agonist (e.g., agonist of a GPCR signaling pathway).

Baselines and controls can be used to adjudge the status of the cellular pathway. Suitable baselines can include, but are not limited to, a sample without the agonist, a sample of infinite dilution of the agonist, the same sample prior to or following sufficiently lengthy time after the addition of the agonist, and other such baselining activities known to those skilled in the art of cell based assays.

Suitable controls can include, but are not limited to, a sample of healthy material from the same patient, a set of samples of healthy material from a sufficient number of patients lacking the disease of interest to derive a normal reference interval, a sample with a similar but different agonist, a cell line of known positive or negative response, a sample treated with the inverse activity of the agonist, a sample of diseased material from one or more patients, and other such positive and negative controls known to those practiced in the art of cell based assays.

### Analysis and Interpretation of Test Results

The test results obtained using the methods described herein can be analyzed and interpreted in a variety of ways to provide information to a clinician and/or a patient. Certain embodiments are set forth as follows.

(i) Diseased Pathway Analysis. This analysis identifies whether diseased pathway activity is found in a patient ex vivo. The analysis will provide physicians with a dynamic evaluation of whether a disease process is present in a patient's diseased cells. In this embodiment, tested pathways can be classified into one of four groups categories: constitutively active, hyperactive, not active at all (hypo-active), or normally active. To determine whether the pathway (e.g., GPCR signaling pathway) is diseased and thus suitable for treatment with a targeted therapy (e.g., RAS node or RTK targeted therapeutic), the pathway activity as determined by the methods described herein for a patient suspected of having the disease is compared to a cut-off value derived from a statistical analysis of that pathway activity in a randomly selected population of patients suspected of having the disease. A drug targeting pathway found to have pathway activity that is abnormal (e.g. above a cut-off delineating abnormal and normal pathway activity) would be expected to disrupt that activity, thereby producing the intended effect in a patient.

(ii) Drug Functionality Analysis. This analysis provides two measures of the functionality of a drug ex vivo.

1) Response Score (RS): The response score characterizes the functional effect that a tested drug (e.g., an inhibitor of a RAS node or RTK signaling) has on the targeted pathway. It can be reported on a 0-1 scale, where a higher score indicates greater drug functionality. 2) Response Score Percentile Ranking (RSPR): RSPR characterizes how a patient's Response Score ranks relative to the scores received by other patients tested with the same agent. For each patient, the percentile of their Response Score within the total group is determined. Once a percentile ranking has been assigned, patients can then be classified into one of three groups: a) below median, b) near median, or c) above median. For certain drugs, a wide variation in patient drug response as measured by a clinical endpoint such as time to progression (TTP) will be mirrored in the variation in Response Scores. Since it is often the case that the TTP period of the 75th percentile patient in a clinical trial is 5-10 times greater than the TTP period of the 25th percentile patient, providing physicians with the relative rank of their patient's response score gives them important interpretive context. For instance, they could estimate the TTP period for an individual patient based on the TTP period of patients in a clinical trial at the percentile range that corresponds to the Response Score percentile of the individual patient.

(iii) Prediction of Likely Clinical Outcome. This analysis reflects the correlation found in a clinical trial between the Response Score and the clinical endpoint for patients tested and observed after receiving the agent in question. With this correlation, it is possible to identify the clinical outcome that is consistent with patients who received a certain Response Score in a clinical trial. For example, if TTP was the clinical outcome measured, a patient's results could be classified into one of three categories.
1) Likely TTP Period—Lowest: Patients falling into this sub-population are likely to experience a TTP period that is well below the median TTP period the entire population of patients would experience.
2) Likely TTP Period—Indeterminate: No assessment is provided for patients who receive a Response Score that falls in this category.
3) Likely TTP Period—Highest: Patients falling into this sub-population are likely to experience a TTP period that is well above the median TTP period the entire group of patients would experience.

Clinicians would use the results of the CELx Profile test as guidance as they determine which drug therapy to select. When a patient's cells are tested with multiple agonists agents and targeted therapeutics, the likely clinical outcome of each drug or combination of drugs can be compared so that the physician can select the drug or combination of drugs with a test result that correlates to the greatest likely clinical outcome.

### Kits

In another aspect of the invention, kits are provided. In certain embodiments, the kit comprises a container for a disease cell sample from an individual subject containing a transport medium; a container for a control cell sample from the individual subject containing a transport medium; a biosensor; a non-transitory computer readable medium having computer executable instructions for converting data from the biosensor into an output, wherein the output shows a change in a cellular physiological response parameter over a defined period of time, wherein the cellular physiological response parameter is cell adhesion or cell attachment, classifying the output as above or below a cutoff value indicating status as a responder or nonresponder and/or classifying the sample as having no response, weakly responsive, and responsive; and generating a report with the classification. In another embodiment, the cellular physiological response parameter is selected from the group consisting of pH, cell adhesion, cell attachment pattern, cell proliferation, cell signaling, cell survival, cell density, cell size, cell shape, cell polarity, O₂, CO₂, glucose, cell cycle, anabolism, catabolism, small molecule synthesis and generation, turnover, and respiration, ATP, calcium, magnesium, and other charged ions, proteins, specific pathway member molecules, DNA and or RNA in various cellular compartments, genomics, and proteomics, post-translational modifications and mechanisms, levels of secondary messenger, cAMP, mRNA, RNAi, microRNAs and other RNA with physiologic function, and combinations thereof.

Types and amounts of disease cell samples are described herein. In certain embodiments, the disease cell sample is a whole cell label free viable cell sample having at least 5,000 cells. In some embodiments, a control cell sample is selected from the group consisting of a disease cell sample from the same subject, a healthy cell sample from the same subject, a healthy cell sample from a subject known to be free from disease, a set of samples of healthy material from a sufficient number of patients lacking the disease of interest to derive a normal reference interval, a cell sample known to respond to the therapeutic agent, a cell sample known not to respond to the therapeutic agent, and combinations thereof.

The containers and the transport medium are designed to maintain cell viability and to minimize cell activation. In certain embodiments, the media and containers are endotoxin free, nonpyrogenic and DNase- and RNase-free. Once obtained the cell samples are maintained in a transport medium that retains the cell viability. Depending on the length of time for transportation to the site of analysis, different media may be employed. In some embodiments, when transportation of the tissue sample may require up to 10 hours, the media has an osmolality of less than 400 mosm/L and comprises Na+, K+, Mg+, Cl-, Ca+2, glucose, glutamine, histidine, mannitol, and tryptophan, penicillin, streptomycin, contains essential amino acids and may additionally contain non-essential amino acids, vitamins, other organic compounds, trace minerals and inorganic salts, serum, cell extracts, or growth factors, insulin, transferrin, sodium selenite, hydrocortisone, ethanolamine, phosphophorylethanoloamine, tridothyronine, sodium pyruvate, L-glutamine, to support the proliferation and plating efficiency of human primary cells. Examples of such a media include Celsior media, Roswell Park Memorial Institute medium (RPMI), Hanks Buffered Saline, and McCoy's 5A, Eagle's Essential Minimal Media (EMEM), Dulbecco's modified Eagle's medium (DMEM), Leibovitz L-15, or modifications thereof for the practice of primary cell care.

Biosensors are described herein. In certain embodiments, a biosensor is selected from the group consisting of a biosensor that detects a cellular parameter selected from the group consisting of, cell adhesion, cell attachment, cell morphology, cell phenotype, cell proliferation, cell signaling, cell density, cell polarity, pH, O₂, CO₂, glucose, and combinations thereof. In some embodiments, the device is an impedance or an optical device. Biosensors may be optionally coated as described herein. In embodiments, a biosensor is selected that measures a change in a physiological parameter associated with the type of therapeutic and/or activator agent as described herein.

In other embodiments, the kit comprises a non-transitory computer readable medium having computer executable instructions for converting data from the biosensor into an output, wherein the output shows a change in a cellular physiological response parameter over a defined period of time, wherein the cellular physiological response parameter is selected from the group consisting of pH, cell adhesion, cell attachment pattern, cell proliferation, cell signaling, cell survival, cell density, cell size, cell shape, cell polarity, O₂, CO₂, glucose, and combinations thereof; classifying the output as a responder or nonresponder and/or no response, weakly responsive, and responsive; and generating a report with the classification.

In other embodiments, the invention provides a computing device or computer readable medium with instructions to implement the methods of the disclosure. The computer readable medium includes non-transitory CD, DVD, flash drive, external hard drive, and mobile device.

The kits and methods described herein can employ the use of a processor/computer system. For example, a general purpose computer system comprising a processor coupled to program memory storing computer program code to implement the method, to working memory, and to interfaces such as a conventional computer screen, keyboard, mouse, and printer, as well as other interfaces, such as a network interface, and software interfaces including a database interface find use one embodiment described herein.

The computer system accepts user input from a data input device, such as a keyboard, input data file, or network interface, or another system, such as the system interpreting, for example, the data generated by the biosensor over a defined period of time, and provides an output to an output device such as a printer, display, network interface, or data storage device. Input device, for example a network interface, receives an input comprising a change in a cellular physiological parameter as described herein and/or quantification of these changes. The output device provides an output such as a display, including one or more numbers and/or a graph depicting the detection and/or quantification of the change in a cellular parameter.

The computer system can be coupled to a data store which stores data generated by the methods described herein. This data is stored for each measurement and/or each subject; optionally a plurality of sets of each of these data types is stored corresponding to each subject. One or more computers/processors may be used, for example, as a separate machine, for example, coupled to computer system over a network, or may comprise a separate or integrated program running on computer system. Whichever method is employed these systems receive data and provide data regarding detection/diagnosis in return.

In some embodiments, the computing device can include a single computing device, such as a server computer. In other embodiments, the computing device can include multiple computing devices configured to communicate with one another over a network (not shown). The computing device can store multiple databases within memory. The databases stored on the computing device can be organized by clinic, practicing clinician, programmer identification code, or any other desired category.

Data from the biosensor can be sent to the remote computing system or another data storage device. The communication process initializes and begins at a start module and proceeds to a connect operation. The connect operation communicatively couples the stored information of the health care provider to the remote computing system, for example, via a cabled connection, a wireless local area network (WLAN or Wi-Fi) connection, a cellular network, a wireless personal area network (WPAN) connection, e.g., BLUETOOTH®, or any desired communication link.

A transfer operation transmits data from the biosensor to the computing device. In an embodiment, the transfer operation encrypts the data before transmitting the data between the devices. The communication process can complete and end at a stop module. Once the biosensor data is transferred to a remote computing device, the data is converted to an output, such as a cell index measurement over time. In certain embodiments, a defined endpoint is selected and is used to classify the cell sample as no response, weakly responsive or responsive as described herein. In embodiments, the status of the analysis of the sample as a responder or nonresponder is communicated back to the health care provider using a similar process over cabled connection, a wireless local area network (WLAN or Wi-Fi) connection, a cellular network, a wireless personal area network (WPAN) connection, e.g., BLUETOOTH®, or any desired communication link.

In certain embodiments, the computer readable storage medium has computer-executable instructions that, when executed by a computing device, cause the computing device to perform steps comprising: converting data from the biosensor into an output, wherein the output shows a change in a cellular physiological response parameter over a defined period of time, wherein the cellular physiological response parameter is selected from the group consisting of pH, cell adhesion, cell attachment pattern, cell proliferation, cell signaling, cell survival, cell density, cell size, cell shape, cell polarity, O₂, CO₂, glucose, and combinations thereof in the presence and/or absence of a therapeutic agent; classifying the output as no response, and responsive at a defined endpoint by comparing the output from biosensor from the cell sample in the presence of the therapeutic agent to the output from biosensor from the cell sample in the absence of the therapeutic agent; and generating a report with the classification. In some embodiments, the computer executable instructions comprise instructions for communicating the classification to a health care provider.

In other embodiments, the computer readable storage medium may include instructions for identifying which pathways are operative in the disease cell sample of the subject. The instructions that when executed by a computing device comprise determining whether there is a difference between the output of the biosensor data from a disease cell sample from a subject treated with an agonist (e.g., an agonist of a GPCR signaling pathway) and the output of the biosensor data from a second disease cell sample from the same subject not treated with the agonist to determine whether the pathway responsive to the agonist is active in the disease cell sample; identifying the presence of the difference in output as an indication of activity of the pathway, and communicating the activity of the pathway to a health care provider. Agonists and their pathways are described herein.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Testing of PI3K Inhibitors and GPCR Signaling Agonists in Cancer Cells that are HER2-negative/PI3K Wild-Type

Biosensor and transducer: A 96-well impendence E-Plate (ACEA, San Diego, Calif.) was placed onto an xCELLigence RTCA MP Station impendence biosensor (ACEA, San Diego, Calif.). The biosensor measured simultaneously the impedance of every well. The change in impedance for a particular well is proportional to the number of cells and type of attachment the cells have with the impedance microplate. Changes in impedance indicate a response to perturbation of these small cell populations.
Coatings: The 96-E-Plate were wells were coated with fibronectin and or collagen. Collagen was purchased from Advanced BioMatrix (Carlsbad, Calif.) and fibronectin from Sigma-Aldrich (St. Louis, Mo.).
Tissue and cell samples: Eighteen HER2-negative/PI3K wild-type breast tumor cell samples were studied.
Signaling pathway activator agent (agonist) and targeted therapeutic pairs:

For each of the 18 tumor cells samples, live cell response to an S1P agonist (signaling pathway activator) and an LPA1 agonist (signaling pathway activator) was measured individually and in combination with two PI3K targeted therapies, alpelisib and IPI-549. From these responses, the net amount of PI3K participation in S1P-initiated and LPA1-initiated signaling was quantified.

Other Reagents: Standard media antibiotics (e.g. penicillin, streptomycin) and other buffers were purchased and used as delivered from ATCC (Manassas, Va., USA) or Life Technologies (Grand Island, N.Y.).
Procedure: For each breast tumor cell sample, seven wells were seeded with approximately 12,750 cells in 120 uL culture media. Twenty microliters of each targeted therapeutic (alpelisib, IPI-549) was added separately to two wells of each patient's cells (four wells total) and 20 microliters of standard media were added to the other three wells of each patient's cell sample 18 hours in advance of addition of the signaling pathway activator agent. Twenty microliters of each signaling pathway agonist (S1P, LPA1) was added separately to the wells of each patient's cells that received a targeted therapeutic agent and separately in one additional well for each patient's set of cells without the targeted therapeutic. A single well of cells that received neither the activating agent or targeted therapeutic served as the control.

Cell Sample Test Configuration

| | Well# | | | | | | |
|---|---|---|---|---|---|---|---|
| | 1 | 2 | 3 | 4 | 5 | 6 | 7 |
| Activating and | None | LPA1 | S1P | Alpelisib + | Alpelisib + | IPI-549 + | IPI-549 + |
| Inhibitor Agent | | | | LPA1 | S1P | LPA1 | S1P |

The impedance recording of attachment and adhesion change was performed at 37° C., 5% CO2. Data was recorded on a continuous basis throughout the test, where the data presented is from the initial baseline level of cell attachment compared to the subsequent effects following the targeted therapeutic agent and the signaling pathway agonist additions on the 18 different patient samples. PI3K targeted therapeutics that generate an output value above a pre-determined cut-off value, i.e., 250 or greater in this example, are ones that would be selected to treat the patient.

### Cut-Off Value

The cut-off was previously determined by characterizing the population distribution of signaling activity levels or output values using a finite mixture model analysis with the aid of statistical analysis software (e.g. mixtools, an R package for analyzing finite mixture models). Two or more groups were identified within the population of cancer patients analyzed; the cut-off value represents the mid-point between the mean output value of one group and the mean output value of the second or other groups is selected.

### Results:

Table 3 presents the results, expressed as an output value, for each of the 72 tests performed using the methods described above. For each of the 18 cell samples, two different GPCR signaling pathways were tested, S1P and LPA1 alone and with two different targeted therapeutics, alpelisib and IPI-549. The output value represents the difference between the amount of cell adhesion change resulting from addition of the S1P and LPA1 agonists (signaling pathway activator agent) and the amount of cell adhesion change resulting from addition of the targeted therapeutic agents, alpelisib and IPI-549. In this example, five of the eighteen patient cell samples tested recorded output values with the targeted therapeutic, IPI-549, above 250. These five patients would thus be selected to receive treatment with IPI-549, even though they lack PI3K mutations.

TABLE 3
Results for a Test of GPCR Signaling Pathway
Activity and PI3K Targeted Therapeutics

| | | | | | Drug Selected |
|---|---|---|---|---|---|
| | Alpelisib | IPI-549 | Alpelisib | IPI-549 | on Basis of Test |
| Patent ID | S1P | S1P | LPA | LPA | Results |
| C152 | 155 | 215 | 85 | 476 | IPI-549 |
| C230 | 109 | 193 | 60 | 360 | IPI-549 |
| C371 | 190 | 194 | 152 | 359 | IPI-549 |
| C270 | 112 | 203 | 197 | 343 | IPI-549 |
| C199 | 131 | 162 | −7 | 276 | IPI-549 |
| C197 | 40 | 167 | −54 | 239 | None |
| C201 | 48 | 77 | 84 | 226 | None |
| C223 | 66 | 208 | 26 | 206 | None |
| C251 | 18 | 25 | 118 | 163 | None |
| C307 | 67 | 36 | 72 | 125 | None |
| C129 | 43 | 65 | 7 | 118 | None |
| C219 | 16 | 18 | 55 | 113 | None |
| C133 | 43 | 43 | 16 | 95 | None |
| C434 | −41 | −14 | −20 | 80 | None |
| C148 | 0 | −14 | 18 | 52 | None |
| C125 | 6 | 1 | 23 | 35 | None |
| C213 | −11 | −16 | 7 | 19 | None |
| C276 | 14 | 38 | 2 | 19 | None |

### Discussion

Standard-of-care guidelines require confirmation of PI3K mutation status before treating breast cancer patients with PI3K targeted therapeutics. Since the patient cell samples tested in this example lack PI3K mutations, they would not be eligible to receive treatment with a PI3K targeted therapeutic; they lack a mutation corresponding to the binding site of the targeted therapy. However, in this example, five of the 18 patients' tumor cell samples recorded output values above a cut-off with a PI3K targeted therapeutic and would thus be selected on the basis of the method described herein to be treated with the corresponding PI3K targeted therapeutic. For these five patients, the targeted therapeutic that is administered using the results of this method is thus different than the targeted therapy current standard-of-care treatment guidelines recommend. This example suggests the importance of measuring the activity of a cancer patient's GPCR signaling pathways since in many cases, the underlying disease mechanism, i.e., a demonstrated specific signaling pathway dysfunction in that patient, is not associated with a corresponding genetic mutation.

This example also demonstrates how the method described herein can evaluate the effect of a targeted therapeutic with a binding site different than the binding site of the activating agent in order to select patients for treatment. In this example, two different GPCR pathways were activated with ligands that bind to their respective receptors (S1P and LPA1) and the effect of targeted therapeutics (alpelisib and IPI-549) that bind to PI3K was measured to provide results that may be used to select patients for treatment with a PI3K inhibitor.

### Example 2: Testing of a HER2 Inhibitor and GPCR Signaling Agonists in Cancer Cells that are HER2-negative/PI3K Wild-Type

Biosensor and transducer: A 96-well impendence E-Plate (ACEA, San Diego, Calif.) was placed onto an xCELLigence RTCA MP Station impendence biosensor (ACEA, San Diego, Calif.). The biosensor measured simultaneously the impedance of every well. The change in impedance for a particular well is proportional to the number of cells and type of attachment the cells have with the impedance microplate. Changes in impedance indicate a response to perturbation of these small cell populations.
Coatings: The 96-E-Plate were wells were coated with fibronectin and or collagen. Collagen was purchased from Advanced BioMatrix (Carlsbad, Calif.) and fibronectin from Sigma-Aldrich (St. Louis, Mo.).
Tissue and cell samples: Eighteen HER2-negative/PI3K wild-type breast tumor cell samples were studied.
Signaling pathway activator agent (agonist) and targeted therapeutic pairs: For each of the 18 tumor cells samples, live cell response to an S1P agonist (signaling pathway activator) and an LPA1 agonist (signaling pathway activator) was measured individually and in combination with a HER2 inhibitor (neratinib). From these responses, the net amount of HER-family pathway participation in S1P-initiated and LPA1-initiated signaling was quantified.
Other Reagents: Standard media antibiotics (e.g. penicillin, streptomycin) and other buffers were purchased and used as delivered from ATCC (Manassas, Va., USA) or Life Technologies (Grand Island, N.Y.).
Procedure: For each breast tumor cell sample, five wells were seeded with approximately 12,750 cells in 120 uL culture media. Twenty microliters of the targeted therapeutic (neratinib) was added separately to two wells of each patient's cells and 20 microliters of standard media were added to the other three wells of each patient's cell sample 18 hours in advance of addition of the signaling pathway activator agent. Twenty microliters of each signaling pathway agonist (S1P, LPA1) was added separately to the one well of each patient's cells that received the targeted therapeutic agent and separately in one additional well for each patient's set of cells without the targeted therapeutic. A single well of cells that received neither the activating agent or targeted therapeutic served as the control.

Cell Sample Test Configuration

| Well # | 1 | 2 | 3 | 4 | 5 |
|---|---|---|---|---|---|
| Activating and | None | LPA1 | S1P | Neratinib + | Neratinib + |
| Inhibitor Agent | | | | LPA1 | S1P |

The impedance recording of attachment and adhesion change was performed at 37° C., 5% CO2. Data was recorded on a continuous basis throughout the test, where the data presented is from the initial baseline level of cell attachment compared to the subsequent effects following the targeted therapeutic agent and the signaling pathway activator agent additions on the 18 different patient samples. Targeted therapeutics that record an output value above a pre-determined cut-off value, 500 or greater in this example, are ones that would be selected to treat the patient.

### Cut-Off Value

The cut-off was previously determined by characterizing the population distribution of signaling activity levels or output values using a finite mixture model analysis with the aid of statistical analysis software (e.g. mixtools, an R package for analyzing finite mixture models). Two or more groups were identified within the population of cancer patients analyzed; the cut-off value represents the mid-point between the mean output value of one group and the mean output value of the second or other groups.

### Results:

Table 4 presents the results, expressed as an output value, for each of the 36 tests performed using the methods described above. For each of the 18 cell samples, two different GPCR signaling pathways were tested, S1P and LPA1, alone and with a HER2 inhibitor, neratinib. The output value represents the difference between the amount of cell adhesion change resulting from addition of the S1P or LPA1 agonists (signaling pathway activator agent) and the amount of cell adhesion change resulting from addition of the targeted therapeutic agent, neratinib. In this example, four of the eighteen patient cell samples tested recorded output values with the targeted therapeutic, neratinib, above 500. These four patients would thus be selected to receive treatment with neratinib, even though they are HER2-negative.

TABLE 4
Results for a Test of GPCR Signaling Pathway
Activity and a HER2 Targeted Therapeutics

| | | | Drug Selected on |
|---|---|---|---|
| | Neratinib | Neratinib | Basis of Test |
| Patent ID | S1P | LPA1 | Results |
| C270 | 301 | 962 | Neratinib |
| C223 | 284 | 666 | Neratinib |
| C197 | 313 | 590 | Neratinib |
| C230 | 289 | 506 | Neratinib |
| C152 | 198 | 482 | None |
| C371 | 270 | 460 | None |
| C199 | 283 | 296 | None |
| C129 | 48 | 247 | None |
| C276 | 11 | 166 | None |
| C133 | 65 | 161 | None |
| C201 | −12 | 110 | None |
| C251 | 16 | 106 | None |
| C219 | −48 | 92 | None |
| C125 | −19 | 54 | None |
| C213 | 41 | 42 | None |
| C148 | −11 | 38 | None |
| C307 | 41 | 38 | None |
| C434 | −1 | 35 | None |

### Discussion

Standard-of-care guidelines require confirmation that patients are HER2-positive before treating patients with HER2 targeted therapeutics. Since the patient cell samples tested in this example are HER2-negative, they would not be eligible to receive treatment with a HER2 targeted therapeutic; they lack the mutation corresponding to the binding site of the targeted therapy. However, in this example, four of the 18 patients' tumor cell samples recorded output values above a cut-off with a HER2 targeted therapeutic and GPCR activated signaling and would thus be selected on the basis of the method described herein to be treated with the corresponding HER2 targeted therapeutic. For these four patients, the targeted therapeutic that is administered using the results of this method described herein is thus different than the targeted therapy current standard-of-care treatment guidelines recommend. This example suggests the importance of measuring the activity of a cancer's patients GPCR signaling pathways since in some cases, the underlying disease mechanism, i.e., a demonstrated specific signaling pathway dysfunction in that patient, is not associated with a corresponding genetic mutation.

As with the previous example, this example demonstrates how the method described herein can evaluate the effect of a targeted therapeutic with a binding site different than the binding site of the activating agent in order to select patients for treatment. In this example, two different GPCR pathways were activated with ligands that bind to their respective receptors (S1P and LPA1) and the effect of a targeted therapeutic (neratinib) that binds to HER1, HER2, and HER3 was measured to provide results that may be used to select patients for treatment with neratinib.

### Example 3: Testing of PI3K Inhibitors and GPCR Signaling Agonists in Cancer Cells that have PI3K Mutations

Biosensor and transducer: A 96-well impendence E-Plate (ACEA, San Diego, Calif.) was placed onto an xCELLigence RTCA MP Station impendence biosensor (ACEA, San Diego, Calif.). The biosensor measured simultaneously the impedance of every well. The change in impedance for a particular well is proportional to the number of cells and type of attachment the cells have with the impedance microplate. Changes in impedance indicate a response to perturbation of these small cell populations.
Coatings: The 96-E-Plate were wells were coated with fibronectin and or collagen. Collagen is purchased from Advanced BioMatrix (Carlsbad, Calif.) and fibronectin from Sigma-Aldrich (St. Louis, Mo.).
Tissue and cell samples: Three breast tumor cell samples with PI3K-alpha mutations were studied.
Signaling pathway activator agent (agonist) and targeted therapeutic pairs: For each of the three breast tumor cells samples, live cell response to an S1P agonist (signaling pathway activator) and an LPA1 agonist (signaling pathway activator) was measured individually and in combination and two PI3K targeted therapies, alpelisib and IPI-549. From these responses, the net amount of PI3K participation in S1P-initiated and LPA1 initiated signaling was quantified.
Other Reagents: Standard media antibiotics (e.g. penicillin, streptomycin) and other buffers were purchased and used as delivered from ATCC (Manassas, Va., USA) or Life Technologies (Grand Island, N.Y.).
Procedure: For each breast tumor cell sample, seven wells were seeded with approximately 12,750 cells in 120 uL culture media. Twenty microliters of each targeted therapeutic (alpelisib, IPI-549) was added separately to two wells of each patient's cells and 20 microliters of standard media were added to the other three wells of each patient's cell sample 18 hours in advance of addition of the signaling pathway activator agent. Twenty microliters of the signaling pathway agonists (S1P, LPA1) was added separately to the wells of each patient's cells that received a targeted therapeutic agent and separately in one additional well for each patient's set of cells without the targeted therapeutic. A single well of cells that received neither the activating agent or targeted therapeutic served as the control.

Cell Sample Test Configuration

| | Well# | | | | | | |
|---|---|---|---|---|---|---|---|
| | 1 | 2 | 3 | 4 | 5 | 6 | 7 |
| Activating and | None | S1P | LPA1 | Alpelisib + | IPI-549 + | Alpelisib + | IPI-549 + |
| Inhibitor Agent | | | | S1P | S1P | LPA1 | LPA1 |

The impedance recording of attachment and adhesion change was performed at 37° C., 5% CO2. Data was recorded on a continuous basis throughout the test, where the data presented is from the initial baseline level of cell attachment compared to the subsequent effects following the targeted therapeutic agent and the signaling pathway activator agent additions on the 18 different patient samples. PI3K targeted therapeutics that record an output value above a pre-determined cut-off value, 250 or greater in this example, are ones that would be selected to treat the patient.

### Cut-Off Value

The cut-off was previously determined by characterizing the population distribution of signaling activity levels or output values using a finite mixture model analysis with the aid of statistical analysis software (e.g. mixtools, an R package for analyzing finite mixture models). Two or more groups were identified within the population of cancer patients analyzed; the cut-off value represents the mid-point between the mean output value of one group and the mean output value of the second or other groups is selected.

### Results:

Table 5 presents the results, expressed as an output value, for each of the 10 tests performed using the methods described above. Two different GPCR signaling pathways were tested, S1P and LPA1 separately and with two PI3K inhibitors, alpelisib and IPI-549. The output value represents the difference between the amount of cell adhesion change resulting from addition of the S1P or LPA1 agonists (signaling pathway activator agent) and the amount of cell adhesion change resulting from addition of the targeted therapeutic agents, alpelisib or IPI-549. In this example, only one of the PI3K-mutated patient cell samples recorded output values above 250 with the PI3K targeted therapeutic. The other two patients, despite having PI3K mutations, had output values below the cut-off and thus would not be selected to receive treatment with a PI3K inhibitor.

TABLE 5
Results for a Test of GPCR Signaling Pathway
Activity and PI3K Targeted Therapeutics

| | | | | | Drug |
|---|---|---|---|---|---|
| | | | | | Selected on |
| | Alpelisib | IPI-549 | Alpelisib | IPI-549 | Basis of Test |
| Patent ID | S1P | S1P | LPA1 | LPA1 | Results |
| CL125 | 338 | 0 | 517.2 | −49.2 | Alpelisib |
| CL21 | 96 | 17 | 142.8 | −30 | None |
| CL142 | 72 | 0 | NT | NT | None |

### Discussion

Standard-of-care guidelines require confirmation of PI3K mutation status before treating breast cancer patients with PI3K targeted therapeutics. Since the patient cell samples tested in this example have PI3K mutations, they would be eligible to receive treatment with a PI3K targeted therapeutic; their mutation corresponds to the binding site of the targeted therapy. However, in this example, two of the three patients' tumor cell samples recorded output values below a cut-off with a PI3K targeted therapeutic and would thus not be selected for treatment with the corresponding PI3K targeted therapeutic on the basis of the method described herein. As with the prior examples, this example suggests the importance of measuring the activity of a cancer patient's GPCR signaling pathways since in many cases, the underlying disease mechanism, i.e., a demonstrated specific signaling pathway dysfunction in that patient, is not associated with a corresponding genetic mutation.

### Example 4: Testing of RAS Node Inhibitors and GPCR Signaling Agonists in Cancer Cells that are HER2-Negative and Wild-Type for PI3K, ERK, MEK, RAF, and mTOR

Dysregulated signaling through GPCRs is implicated in oncogenic RAS signaling in breast cancer (BC). The complex regulatory mechanisms that link RAS nodes can trigger an adaptive response (e.g. resistance) when a single RAS node is inhibited, regardless of the mutational status of the node. Inhibition of multiple RAS nodes may thus be required to achieve durable antitumor responses. To identify patients with dysregulated RAS signaling tumors that may respond to RAS node inhibitors, an assay using an impedance biosensor was developed. This Example tested GPCR-initiated signaling activity and the role(s) of PI3K, ERK, MEK, mTOR, RAF, and BCL in transducing this activity in live tumor cells.

Biosensor and transducer: A 96-well impendence E-Plate (ACEA, San Diego, Calif.) was placed onto an xCELLigence RTCA MP Station impendence biosensor (ACEA, San Diego, Calif.). The biosensor measured simultaneously the impedance of every well. The change in impedance for a particular well is proportional to the number of cells and type of attachment the cells have with the impedance microplate. Changes in impedance indicate a response to perturbation of these small cell populations.
Coatings: The 96-E-Plate were wells were coated with fibronectin and or collagen. Collagen was purchased from Advanced BioMatrix (Carlsbad, Calif.) and fibronectin from Sigma-Aldrich (St. Louis, Mo.).
Tissue and cell samples: 28 HER2-negative/PI3K//ERK/MEK/RAF/mTOR Wild-Type breast tumor cell samples were studied.
Signaling pathway activator agent (agonist) and targeted therapeutic pairs: For each of the 28 tumor cells samples, live cell response to an LPA1 agonist (signaling pathway activator) was measured individually and in combination with a PI3K-α inhibitor (GDC-0077), a pan-PI3K inhibitor (copanlisib), a pan-PI3K/mTOR inhibitor (gedatolisib), a Bcl-xL/Bcl-2 inhibitor (navitoclax), a MEK inhibitor (trametinib), an ERK inhibitor (ravoxertinib), and a RAF inhibitor (PLX7904). From these responses, the net amount of LPA1-initiated signaling involving PI3K-α, Class 1 PI3K-isoforms (pan-PI3K), Class 1 PI3K-isoforms and mTOR, Bcl-xL and Bcl-2, MEK, ERK, and RAF, individually and in combination, was quantified.
Other Reagents: Standard media antibiotics (e.g. penicillin, streptomycin) and other buffers were purchased and used as delivered from ATCC (Manassas, Va., USA) or Life Technologies (Grand Island, N.Y.).
Procedure: For each breast tumor cell sample, 12 wells were seeded with approximately 12,750 cells in 120 microliters of culture media. Twenty microliters of GDC-0077, copanlisib, gedatolisib, navitoclax, trametinib, ravoxertinib, and PLX7904 alone and 20 microliters of a combination of gedatolisib and trametinib, gedatolisib and navitoclax, gedatolisib and ravoxertinib, and gedatolisib and PLX7904, was added separately to 10 wells of each patient's cells, and 20 microliters of standard media were added to the other two wells of each patient's cell sample 18 hours in advance of addition of the signaling pathway activator agent. Twenty microliters of the signaling pathway agonist (LPA1) was added separately to the wells of each patient's cells that received a targeted therapeutic agent and separately in one additional well for each patient's set of cells without the targeted therapeutic. A single well of cells that received neither the activating agent or targeted therapeutic served as the control.
The impedance recording of attachment and adhesion change was performed at 37° C., 5% CO2. Data was recorded on a continuous basis throughout the test, where the data presented is from the initial baseline level of cell attachment compared to the subsequent effects following the targeted therapeutic agent and the signaling pathway agonist additions on the 28 different patient samples. The single targeted therapeutics and combination of targeted therapeutics that generate an output value above a pre-determined cut-off value, i.e., 250 or greater in this example, are ones that would be selected to treat the patient.

### Cut-Off Value

The cut-off was previously determined by characterizing the population distribution of signaling activity levels or output values using a finite mixture model analysis with the aid of statistical analysis software (e.g. mixtools, an R package for analyzing finite mixture models). Two or more groups were identified within the population of cancer patients analyzed; the cut-off value represents the mid-point between the mean output value of one group and the mean output value of the second or other groups is selected.

### Results:

Table 4 presents the results, expressed as an output value, for each of the tests performed using the methods described above. For each of the 28 cell samples, LPA1 was tested alone and with different targeted therapeutics as single agents (GDC-0077, copanlisib, gedatolisib, navitoclax, trametinib, ravoxertinib, PLX7904) and with combination agents (gedatolisib and trametinib, gedatolisib and navitoclax, gedatolisib and ravoxertinib, gedatolisib and PLX7904). The output value represents the difference between the amount of cell adhesion change resulting from addition of the LPA1 agonist (signaling pathway activator agent) and the amount of cell adhesion change resulting from addition of the targeted therapeutic agent(s). In this example, 14 of the 28 patient cell samples tested recorded output values with at least one of the targeted therapeutic(s) above 250. No patients would be selected to receive treatment with trametinib, GDC-0077, ravoxertinib as single agents. Five patients would be selected to receive treatment with gedatolisib, four would be selected to receive treatment with copanlisib, one patient would be selected to receive treatment with PLX7904, six patients would be selected to receive treatment with gedatolisib and PLX7904, seven patients would be selected to receive treatment with gedatolisib and ravoxertinib, nine patients would be selected to receive treatment with gedatolisib and trametinib, and 14 patients would be selected to receive treatment with gedatolisib and navitoclax.

TABLE 4
Results for a Test of GPCR Signaling Pathway Activity and RAS Targeted Therapeutics

| Patient | | | | | | | gedatolisib + | gedatolisib + | gedatolisib + | gedatolisib + |
|---|---|---|---|---|---|---|---|---|---|---|
| Sample | gedatolisib | trametinib | GDC-0077 | Copanlisib | ravoxertinib | PLX7904 | navitoclax | LX7904 | ravoxertinib | trametinib |
| C3730.01.9 | 494 | −204 | −113 | 511 | −300 | −173 | 857 | 528 | 526 | 566 |
| C3311.01.10 | 216 | 94 | 14 | 430 | −67 | −17 | 367 | 242 | 305 | 365 |
| CL021.01.869 | 353 | −226 | −466 | −259 | −226 | 19 | 504 | 290 | 127 | 305 |
| C3337.01.6 | 336 | 113 | 139 | 240 | 53 | 58 | 456 | 358 | 372 | 384 |
| C2991.01.7 | 396 | −70 | 55 | 310 | 10 | 53 | 504 | 410 | 422 | 406 |
| C1816.01.6 | 84 | 29 | −22 | 374 | 96 | 158 | 382 | 394 | 377 | 257 |
| C3377.01.2 | 113 | 149 | −161 | 86 | 0 | −58 | 360 | 84 | 173 | 293 |
| C3873.01.4 | 298 | −72 | 122 | 199 | 43 | 89 | 370 | 310 | 290 | 170 |
| C1986.01.10 | 101 | 161 | 62 | 170 | 26 | −19 | 300 | 46 | 226 | 190 |
| C3091.01.13 | 204 | 50 | 60 | 156 | 67 | 50 | 264 | 190 | 209 | 218 |
| C1918.01.5 | 206 | 65 | 38 | 113 | 65 | −5 | 281 | 235 | 185 | 214 |
| C4005.01.2 | 216 | 36 | −12 | 125 | 12 | −22 | 322 | 209 | 218 | 257 |
| C3101.01.6 | 149 | 101 | 0 | 70 | −12 | 262 | 266 | 151 | 161 | 185 |
| C2169.01.10 | 242 | 67 | 34 | 199 | 14 | 14 | 293 | 240 | 252 | 262 |
| C2483.01.6 | 130 | 132 | 113 | 113 | 12 | 67 | 77 | 125 | 142 | 178 |
| C2792.01.2 | 137 | 48 | 43 | 98 | 24 | 5 | 233 | 146 | 120 | 127 |
| C3507.01.5 | 70 | −29 | −58 | 94 | −24 | 46 | 204 | 89 | 55 | 101 |
| C1838.01.155 | 50 | 31 | 38 | 10 | 108 | −91 | 50 | 144 | 72 | 17 |
| C2697.01.4 | 103 | 72 | 31 | 103 | 55 | −2 | 185 | 122 | 125 | 146 |
| C2693.01.2 | 86 | −60 | −24 | 46 | −55 | −10 | 173 | 89 | 98 | 94 |
| C2167.01.4 | 132 | −7 | −12 | 67 | 7 | 22 | 218 | 113 | 156 | 156 |
| C2748.01.4 | 72 | 79 | 65 | 55 | 60 | 14 | 137 | 94 | 58 | 120 |
| C2868.01.6 | 79 | −43 | −24 | 58 | −36 | −12 | 146 | 84 | 89 | 108 |
| C2090.01.14 | 137 | 19 | 36 | 103 | 48 | −38 | 149 | 115 | 134 | 130 |
| C2794.01.9 | 38 | −14 | −89 | 14 | −24 | 36 | 130 | 34 | 62 | 77 |
| C3957.01.6 | 74 | 10 | −10 | 53 | −5 | −22 | 130 | 65 | 60 | 84 |
| C2729.01.4 | 103 | 26 | 34 | 72 | 10 | −2 | 118 | 84 | 106 | 106 |
| C3132.01.13 | 65 | 10 | 0 | 38 | −22 | 22 | 108 | 62 | 36 | 34 |

### Discussion

Standard-of-care guidelines require confirmation of PJ3K/ERK/MEK/RAF/mTOR Wild-Type mutation status before treating breast cancer patients with corresponding targeted therapeutics. Since the patient cell samples tested in this example lacked PJ3K/ERK/MEK/RAF/mTOR mutations, they would not be eligible to receive treatment with a PJ3K/ERK/MEK/RAF/mTOR targeted therapeutics alone or in combination; they lack a mutation corresponding to the binding site of the targeted therapy. However, in this example, 14 of the 28 patients' tumor cell samples recorded output values above a cut-off with a one of the targeted therapeutic(s) and would thus be selected on the basis of the method described herein to be treated with the corresponding targeted therapeutic. For these 14 patients, the targeted therapeutic that is administered using the results of this method is thus different than the targeted therapy current standard-of-care treatment guidelines recommend. This example suggests the importance of measuring the activity of a cancer patient's GPCR signaling pathways since in many cases, the underlying disease mechanism, i.e., a demonstrated specific signaling pathway dysfunction in that patient, is not associated with a corresponding genetic mutation.

This example also demonstrates how the method described herein can evaluate the effect of a targeted therapeutic with a binding site different than the binding site of the activating agent in order to select patients for treatment. In this example, a GPCR pathway was activated with ligands that bind to its receptor (LPA1) and the effect of targeted therapeutics (GDC-0077, copanlisib, gedatolisib, navitoclax, trametinib, ravoxertinib, PLX7904, gedatolisib and trametinib, gedatolisib and navitoclax, gedatolisib and ravoxertinib, gedatolisib and PLX7904) that bind to a RAS node was measured to provide results that may be used to select patients for treatment with a RAS node inhibitor.

1. A method of selecting a human subject diagnosed with cancer for treatment with a RAS node targeted therapeutic, the method comprising:
culturing a sample of viable cancer cells obtained from the subject;
contacting (1) a first portion of the sample with an agonist of G-protein coupled receptor (GPCR) signaling and an inhibitor of a RAS node, and (2) a second portion of the sample with the agonist alone;
continuously measuring cell adhesion or attachment of the viable cancer cells in the first and second portions;
determining by mathematical analysis of the continuous measurements an output value that characterizes whether a change in cell adhesion or attachment has occurred in the first portion compared to the second portion; and
selecting the subject for treatment with a RAS node targeted therapeutic which inhibits the same RAS node as the inhibitor if the output value that characterizes the change in cell adhesion or attachment is equal to or greater than a pre-determined cut-off value, or if the output value percentage is equal to or greater than 30%.

2. The method of claim 1, further comprising
administering to the subject a RAS node targeted therapeutic which inhibits the same RAS node as the inhibitor if the output value that characterizes the change in cell adhesion or attachment is equal to or greater than a pre-determined cut-off value, or if the output value percentage is equal to or greater than 30%.

3. The method of claim 1, wherein the GPCR is a lysophospholipid GPCR, such as an LPA receptor (LPAR) or a S1P receptor (S1PR).

4. (canceled)

5. The method of claim 3, wherein:
(a) the LPAR is selected from the group consisting of LPAR1, LPAR2, LPAR3, LPAR4, LPAR5, and LPAR6, or
(b) the S1PR is selected from the group consisting of S1PR1, S1PR2, S1PR3, S1PR4, and S1PR5.

6-7. (canceled)

8. The method of claim 1, wherein the agonist is a protein, peptide, lipid, nucleic acid, metabolite, ligand, reagent, organic molecule, signaling factor, growth factor, biochemical, or combinations thereof.

9. The method of claim 1, wherein the agonist is lysophosphatidic acid (LPA), FAP-10, FAP-12, OMPT, sphingosine-1-phosphate (S1P), FTY720, BAF312, A971432, ceralifimod, CS2100, CYM50260, CYM50308, CYM5442, CYM5520, CYM5541, GSK2018682, RP001 hydrochloride, SEW2871, TC-G1006, or TC-SP14.

10-12. (canceled)

13. The method of claim 1, wherein the RAS node is selected from the group consisting of: phosphoinositide 3 kinase (PI3K), ERK, MEK, mTOR, RAF, BCL, and a combination thereof.

14. The method of claim 1, wherein the first portion of the sample is contacted with two or more inhibitors, each of which inhibits a different RAS node.

15. The method of claim 14, wherein the two or more inhibitors inhibit a combination of RAS nodes selected from the group consisting of:
(a) PI3K, mTOR, and BCL,
(b) PI3K, mTOR, and RAF,
(c) PI3K, mTOR, and ERK, and
(d) PI3K, mTOR, and MEK.

16. (canceled)

17. The method of claim 1, wherein the inhibitor is a PI3K inhibitor, an ERK inhibitor, a MEK inhibitor, an mTOR inhibitor, a RAF inhibitor, or a Bcl-xL inhibitor.

18. The method of claim 17, wherein the PI3K inhibitor selectively inhibits:
(a) the p110α catalytic subunit of PI3K,
(b) the p110β catalytic subunit of PI3K,
(c) the p110γ catalytic subunit of PI3K,
(d) the p110δ subunit of PI3K,
(e) more than one isoform of the p110 subunit of PI3K, and/or
(f) all isoforms of the p110 subunit of PI3K.

19-24. (canceled)

25. The method of claim 17, wherein:
(a) the PI3K inhibitor is selected from the group consisting of: wortmannin, LY294002, hibiscone C, Idelalisib (GS-1101, CAL-101), Copanlisib, Duvelisib, Alpelisib, (BYL719), Taselisib (GDC-0032), GDC-0077, Gedatolisib, Perifosine, Idealisib, Pilaralisib (XL147), Buparlisib (BKM120), Duvelisib, Umbralisib, PX-866, Dactolisib, CUDC-907, Voxtalisib, ME-401, IPI-549, SF1126, RP6530, INK1117, Pictilisib (GDC-0941), Palomid 529, SAR260301, GSK1059615, GSK2636771, CH5132799, CZC24832, AZD6482, AZD8835, WX-037, AZD8186, KA2237, CAL-120, AMG-319, AMG-511, HS-173, INCB050465, INCB040093, TGR-1202, ZSTK474, PWT33597, IC87114, TG100-115, TGX221, CAL263, RP6530, PI-103, GNE-477, IPI-145, BAY 80-6946, BAY1082439. PX866, BEZ235, MKM120, MLN1117, SAR245408, AEZS-136, serabelisib (TAK-117), gedatolisib, omipalisib, and pilarlisib;
(b) the ERK inhibitor is selected from the group consisting of ravoxertinib, SCH772984, SCH900353 (MK8353), ulixertinib, AZD0364 (ATG017), VX-11e (VTX-113), CC-90003, LY3214996, FR180204, E6201, ASN007, and GDC0994,
(c) the MEK inhibitor is selected from the group consisting of trametinib, binimetinib, pimasertib, cobimetinib, PD901, U0126, selumetinib, PD325901, TAK733, CI-1040 (PD184352), PD198306, PD334581, PD98059, and SL327,
(d) the mTOR inhibitor is selected from the group consisting of gedatolisib, omipalisib, sirolimus, everolimus, temsirolimus, dactolisib, AZD8055, ABTL-0812, PQR620, GNE-493, KU0063794, torkinib, ridaforolimus, sapanisertib, voxtalisib, torin 1, torin 2, OSI-027, PF-04691502, apitolisib, GSK1059615, WYE-354, vistusertib, WYE-125132, BGT226, palomid 529, WYE-687, WAY600, GDC-0349, XL388, bimiralisib (PQR309), onatasertib (CC-223), samotolisib, RMC-5552, and GNE-477,
(e) the RAF inhibitor is selected from the group consisting of PLX7904, GDC-0879, belvarafenib (GDC-5573), SB590885, encorafenib, RAF265, RAF709, dabrafenib (GSK2118436), TAK-632, TAK580, PLX-4720, CEP-32796, sorafenib, vemurafenib (PLX-4032), AZ-628, GW5074, ZM-336372, NVP-BHG712, CEP32496, PLX4032, PF-0728489, and LGX-818, or
(f) the Bcl-xL inhibitor is selected from the group consisting of navitoclax (ABT-263), GDC-0199 (ABT-199), sabutoxlax (B1-97C1), ABT-737, AT101, TW037, A1331852, BXI-61, and BXI-72.

26-37. (canceled)

38. The method of claim 1, wherein the cancer is selected from the group consisting of breast cancer, lung cancer, colorectal cancer, bladder cancer, kidney cancer, ovarian cancer and leukemia.

39-40. (canceled)

41. The method of claim 1, wherein the cancer cells obtained from the subject express wild-type ERK, wild-type MEK, wild-type RAF, wild-type mTOR, wild-type Bcl-xL, and/or wild-type PI3K enzyme.

42-46. (canceled)

47. A method of selecting a human subject diagnosed with cancer for treatment with a receptor tyrosine kinase (RTK) targeted therapeutic, the method comprising:
culturing a sample of viable cancer cells obtained from the subject;
contacting (1) a first portion of the sample with an agonist of G-protein coupled receptor (GPCR) signaling and an inhibitor of RTK signaling, and (2) a second portion of the sample with the agonist alone;
continuously measuring cell adhesion or attachment of the viable cancer cells in the first and second portions;
determining by mathematical analysis of the continuous measurements an output value that characterizes whether a change in cell adhesion or attachment has occurred in the first portion compared to the second portion; and
selecting the subject for treatment with an RTK targeted therapeutic which inhibits the same RTK as the inhibitor if the output value that characterizes the change in cell adhesion or attachment is equal to or greater than a pre-determined cut-off value, or if the output value percentage is equal to or greater than 30%.

48. The method of claim 47, further comprising
administering to the subject an RTK targeted therapeutic which inhibits the (RTK as the inhibitor if the output value that characterizes the change in cell adhesion or attachment is equal to or greater than a pre-determined cut-off value, or if the output value percentage is equal to or greater than 30%.

49. The method of claim 47, wherein the GPCR is a lysophospholipid GPCR, such as an LPA receptor (LPAR) or an S1P receptor (S1PR) selected from the group consisting of: LPAR1, LPAR2, LPAR3, LPAR4, LPAR5, LPAR6, S1PR1, S1PR2, S1PR3, S1PR4, and S1PR5.

50-53. (canceled)

54. The method of claim 47, wherein the agonist is a protein, peptide, lipid, nucleic acid, metabolite, ligand, reagent, organic molecule, signaling factor, growth factor, biochemical, or combinations thereof.

55. The method of claim 47, wherein the agonist is lysophosphatidic acid (LPA), FAP-10, FAP-12, OMPT, sphingosine-1-phosphate (S1P), FTY720, BAF312, A971432, ceralifimod, CS2100, CYM50260, CYM50308, CYM5442, CYM5520, CYM5541, GSK2018682, RP001 hydrochloride, SEW2871, TC-G1006, or TC-SP14.

56-58. (canceled)

59. The method of claim 47, wherein the RTK is selected from a member of the EGFR/ERBB, MuSK, HGFR, NGFR, FGFR, IR, CCK, EphR, RYK, RET, ROS, PDGFR, DDR, LTK, VEGFR, TIE, AXL, ROR, LMR, or RTK106 family.

60-61. (canceled)

62. The method of claim 47, wherein the RTK inhibitor is selected from the group consisting of: erlotinib, gefitinib, lapatinib, vandetanib, afatinib, panitumumab, cetuximab, brigatinib, icotinib, osimertinib, neratinib, zalutumumab, nimotuzumab, matuzumab, pertuzumab, trastuzumab, dacomitinib, acomitinib, BIBW2992, tesevatinib, amuvatinib, necitumumab, REGN955, MM-151, nazartinib, ASP8273, olmutinib, TDM1, MEDI4276, ZW25, ZW33, tucatinib, rociletinib, ibrutinib, DS-8201, TAS07828, XMT-1522, TAK-788, Sym013, LIM716, seribantumab, AMG888, lumretuzumab, PF-06804103, ARX788, poziotinib, pyrotinib, duligotuzuman, MCLA-128, MM-111, cabozantinib, tivantinib, crizotinib (PF-2341066), tepotinib, capmatinib, savolitinib, K252a, SU11274, PHA-665752, ARQ197, foretinib, SGX523, MP470, AV229, AMG102, CGEN241, DN30, OA5D5, rilotumumab, onartuzumab, SAR125844, embetuzumab, ABBV-399, sym015, ficlatuzumab, merestinib, JNJ-61186372, altiratinib, Indo5, BMS-754807, BMS-777607, glesatinib, CEP-751, ANA-12, cyclotraxin B, gossypetin, entrectinib, larotrectinib, LOXO-101, dovotinib, lenvatinib, ponatinib, regorafenib, lucitanib, cediranib, intedanib, brivanib, PD173074, AZD4547, BGJ398, JNJ42756493, GP369, BAY1187982, MFGR1877S, FP1039, pazopanib, erdafitinib, Debio-1347, B-701, fisogatinib, FIIN-2, FIIN-3, BLU9931, LY2874455, LY3076226, sunitinib, AG538, AG1024, NVP-AEW541, figitumumab, linsitinib, dalotuzumab, MEDI-573, teprotumumab, ganitumab, ceritinib, MM-141, cofetuzumab pelidotin (PF-06647020), dasatinib, nilotinib, NVP-BHG712, sitravatinib, ALW-II-41-27, JI-101, 123C4, sorafenib, apatinib, AST487, alectinib, dovitinib, crizotinib, lorlatinib, TPX-0005, DS-6051b, imatinib, linifanib, KTN0182A, gilteritinib, quizartinib, midostaurin, lestaurtinib, ripretinib, masitinib, avapritinib, pexidartinib, telatinib, motesanib, PLX7486, ARRY386, JNJ-40346527, BLZ945, emactuzumab, AMG820, IMC-CS4, cabiralizumab, CHMFL-KIT-033, SU14813, Ki20227, OSI-930, flumatinib, toceranib, AZD3229, AC710, AZD2932, ICK03, PLX647, c-Kit-IN-3, vatalanib, bevacizumab, rebastinib, BAY-826, bemcentinib, R428 (BGB324), YW327.6S2, GL2I.T, TP-0903, LY2801653, bosutinib, MGCD265, ASP2215, SGI-7079, BGB324, HuMax-AXL-ADC, and UC-961.

63-64. (canceled)

65. The method of claim 47, wherein the cancer is selected from the group consisting of breast cancer, lung cancer, colorectal cancer, bladder cancer, kidney cancer, ovarian cancer and leukemia.

66-70. (canceled)

71. A method of determining whether a human subject diagnosed with cancer has abnormally active G-protein coupled receptor (GPCR) signaling comprising,
culturing a sample comprising viable cancer cells obtained from the subject;
contacting the sample with an agonist of a GPCR signaling pathway;
continuously measuring cell adhesion or attachment of the viable cancer cells in a portion of the sample contacted with the agonist relative to a portion of the sample that has not been contacted with the agonist; and
determining by mathematical analysis of the continuous measurements sensitivity of the sample to the agonist and an output value for the agonist that characterizes whether a change in cell adhesion or attachment has occurred in the portion of the sample contacted with the agonist, as compared to the portion of the sample not contacted with the agonist, wherein the GPCR signaling pathway is considered abnormally active when the output value for the agonist is equal to or greater than a pre-determined cut-off value.

72. A method of determining whether a human subject diagnosed with cancer has abnormally active G-protein coupled receptor (GPCR) signaling comprising,
culturing a sample comprising viable cancer cells obtained from the subject;
contacting the sample with an agonist of a GPCR signaling pathway, wherein a portion of the sample is contacted with a higher concentration of the agonist and a portion of the sample is contacted with a lower concentration of the agonist;
continuously measuring cell adhesion or attachment of the viable cancer cells in the portion of the sample contacted with a higher concentration of the agonist, relative to the portion of the sample contacted with the lower concentration of the agonist; and
determining by mathematical analysis of the continuous measurements the sensitivity of the signaling pathway to the agonist, wherein the GPCR signaling pathway is considered abnormally active when the signaling pathway is ultra-sensitive to the agonist.

73. The method of claim 71, wherein the GPCR signaling pathway is a lysophospholipid GPCR signaling pathway, wherein the lysophospholipid GPCR is optionally a lysophosphatidic acid (LPA) receptor or a sphingosine 1-phosphate (S1P) receptor.

74-78. (canceled)
